(19) United States
(12) Patent Application Publication
(10) Pub. No.: US 2016/0293843 A1
(43) Pub. Date: Oct. 6, 2016

(54) MATERIAL FOR AN ORGANIC ELECTROLUMINESCENT DEVICE AND ORGANIC ELECTROLUMINESCENT DEVICE USING THE SAME

(71) Applicant: Samsung Display Co., Ltd., Yongin-si (KR)

(72) Inventor: Hiroaki ITOI, Yokohama (JP)

(21) Appl. No.: 15/015,090

(22) Filed: Feb. 3, 2016

(30) Foreign Application Priority Data
Mar. 31, 2015 (JP) 2015-070934

(51) Int. Cl. H01L 51/00; C07D 333/76; C07D 307/91

(52) U.S. Cl. CPC H01L 51/006; H01L 51/0061; C07D 307/91; C07D 333/76; H01L 51/5056

(57) ABSTRACT

Embodiments of the present disclosure are directed toward a material for an organic electroluminescent device (represented by Formula 1) and a device using the same:

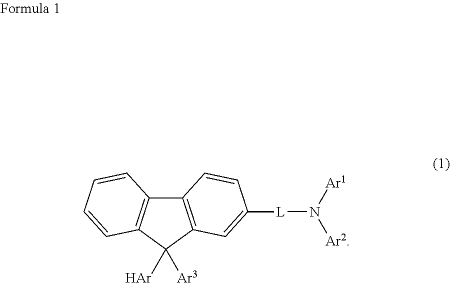

Ar¹ and Ar² may be a substituted or unsubstituted aryl group having 6 to 30 carbon atoms or a substituted or unsubstituted heteroaryl group having 5 to 30 carbon atoms, Ar³ may be a substituted or unsubstituted aryl or heteroaryl group as above or an alkyl group having 1 to 6 carbon atoms, HAr may be a substituted or unsubstituted heteroaryl group having 5 to 30 carbon atoms, L may be selected from a direct linkage, a substituted or unsubstituted arylene group having 6 to 30 carbon atoms, and a substituted or unsubstituted heteroarylene group having 5 to 30 carbon atoms, and HAr and Ar³ may be different from each other.

### CROSS-REFERENCE TO RELATED APPLICATION

This application claims priority to and the benefit of Japanese Patent Application No. 2015-070934, filed on Mar. 31, 2015 in the Japan Patent Office, the entire content of which is incorporated herein by reference.

### BACKGROUND

1. Field

One or more aspects of embodiments of the present disclosure are related to a material for an organic electroluminescent device, and an organic electroluminescent device including the same.

2. Description of the Related Art

Organic electroluminescent (EL) displays are currently being actively developed. Unlike liquid crystal displays, etc. organic EL displays are so-called self-luminescent displays that function by recombining holes and electrons from an anode and a cathode in an emission layer to generate excitons. Light is emitted by a luminescent organic compound in the emission layer.

An example organic EL device includes an anode, a hole transport layer on the anode, an emission layer on the hole transport layer, an electron transport layer on the emission layer, and a cathode on the electron transport layer. Holes injected from the anode move via the hole transport layer to the emission layer. Electrons injected from the cathode move via the electron transport layer to the emission layer. When the holes and electrons injected into the emission layer are recombined, excitons are generated in the emission layer. The organic EL device emits light using energy generated by the radiative decay of the excitons. Configurations of the organic EL device are not limited to the above example, and may be diversely modified.

When organic EL devices are used in display apparatuses, the organic EL devices must exhibit high emission efficiencies and long lifetimes (e.g., life spans). However, driving voltages are high and emission efficiencies and lifetimes are insufficient in many organic EL devices, for example, those in the blue emission region. Methods of increasing the normalization and stabilization of the hole transport layer have been examined as strategies for increasing the efficiencies and lifetimes of organic EL devices.

Many aromatic amine compounds are available as hole transport materials for use in a hole transport layer. For example, a monoamine derivative substituted with a heteroaryl ring at position 9 of a fluorenyl group has been suggested as a useful material for increasing the life of an organic EL device. However, issues related to resolving the emission efficiency and life of the device remain, and it is difficult to say that an organic EL device using this material has a sufficient lifetime.

Accordingly, further developments on material for an organic EL device having a long lifetime and an organic EL device using the same are required.

### SUMMARY

One or more aspects of embodiments of the present disclosure are directed toward a material for an organic EL device having a long lifetime, and an organic EL device using the same.

One or more embodiments of the present disclosure relate to a material for an organic electroluminescent device and an organic electroluminescent device using the same, and to a material for an organic electroluminescent device having a high emission efficiency and long lifetime, and an organic electroluminescent device using the same.

An embodiment of the present disclosure provides a material for an organic EL device having a long lifetime, and an organic EL device using the same in at least one laminated layer between an emission layer and an anode.

An embodiment of the present disclosure provides a material for an organic EL device, represented by the following Formula 1:

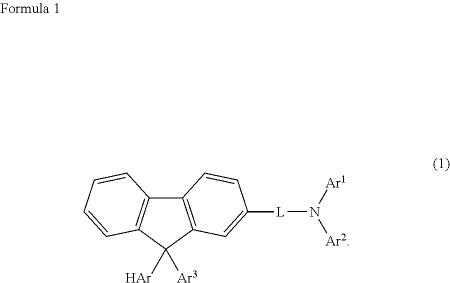

In Formula 1, Ar¹ and Ar² may each independently be selected from a substituted or unsubstituted aryl group having 6 to 30 carbon atoms for forming a ring, and a substituted or unsubstituted heteroaryl group having 5 to 30 carbon atoms for forming a ring, Ar³ may be selected from a substituted or unsubstituted aryl group having 6 to 30 carbon atoms for forming a ring, a substituted or unsubstituted heteroaryl group having 5 to 30 carbon atoms for forming a ring, and an alkyl group having 1 to 6 carbon atoms, HAr may be a substituted or unsubstituted heteroaryl group having 5 to 30 carbon atoms for forming a ring, L may be selected from a direct linkage, a substituted or unsubstituted arylene group having 6 to 30 carbon atoms for forming a ring, and a substituted or unsubstituted heteroarylene group having 5 to 30 carbon atoms, and HAr and Ar³ may be different from each other.

In one embodiment, Ar¹ to Ar³ may each independently be a substituted or unsubstituted aryl group having 6 to 30 carbon atoms for forming a ring.

In one embodiment, HAr may be selected from a dibenzofuryl group and a dibenzothienyl group.

The material for an organic EL device according to an embodiment of the present disclosure may include a heteroaryl group and a substituent different from the heteroaryl group at position 9 of a fluorenyl group, and the fluorenyl group is combined or coupled with an amine, such that the entire molecule is substantially asymmetric, and the material may be more amorphous. Accordingly, charge transport may become smooth (e.g., may be improved), and the lifetime of the organic EL device may be improved.

In an embodiment of the present disclosure, an organic EL device includes the material for an organic EL device in at least one layer.

The organic EL device, according to an embodiment of the present disclosure, includes the material for an organic EL device in at least one layer, and the organic EL device may attain a long lifetime.

In an embodiment of the present disclosure, an organic EL device includes the material for an organic EL device in at least one laminated layer between an emission layer and an anode.

The organic EL device, according to an embodiment of the present disclosure, includes the material for an organic EL device in at least one laminated layer between the emission layer and the anode, and the organic EL device may attain a long lifetime.

The organic EL device according to an embodiment of the present disclosure, includes the material for an organic EL device in a hole transport layer, and the organic EL device may attain a long lifetime.

### BRIEF DESCRIPTION OF THE DRAWINGS

The accompanying drawings are included to enable further understanding of the present disclosure, and are incorporated in and constitute a part of this specification. The drawings illustrate example embodiments of the present disclosure and, together with the description, serve to explain principles of the present disclosure. In the drawings:

FIG. 1 is a schematic view showing an organic EL device according to an embodiment of the present disclosure; and

FIG. 2 is a schematic view showing an organic EL device according to another embodiment of the present disclosure.

### DETAILED DESCRIPTION

Hereinafter, the material for an organic EL device and the organic EL device including the same, according to an embodiment of the present disclosure, will be described with reference to the accompanying drawings. The material for an organic EL device and the organic EL device including the same may, however, be embodied in different forms and should not be construed as being limited to the embodiments set forth herein. In the drawings, like reference numerals refer to like elements or elements having like functions throughout, and repeated explanation thereof will not be provided.

The thickness of layers, films, panels, regions, etc., may be exaggerated in the drawings for clarity. It will be understood that when an element such as a layer, film, region, or substrate is referred to as being “on” another element, it can be directly on the other element or intervening elements may also be present. In contrast, when an element is referred to as being “directly on” another element, no intervening elements are present.

The material for an organic EL device according to an embodiment of the present disclosure may be an amine compound represented by the following Formula 1:

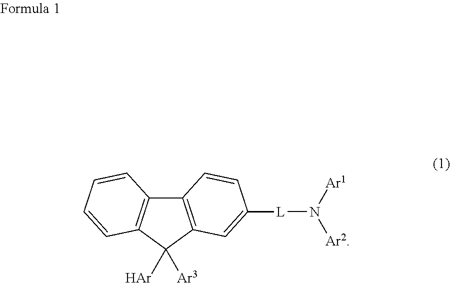

In Formula 1, Ar¹ and Ar² may each independently be selected from a substituted or unsubstituted aryl group having 6 to 30 carbon atoms for forming a ring, and a substituted or unsubstituted heteroaryl group having 5 to 30 carbon atoms for forming a ring, and Ar³ may be selected from a substituted or unsubstituted aryl group having 6 to 30 carbon atoms for forming a ring, a substituted or unsubstituted heteroaryl group having 5 to 30 carbon atoms for forming a ring, and an alkyl group having 1 to 6 carbon atoms. HAr may be a substituted or unsubstituted heteroaryl group having 5 to 30 carbon atoms for forming a ring. L may be selected from a direct linkage, a substituted or unsubstituted arylene group having 6 to 30 carbon atoms for forming a ring, and a substituted or unsubstituted heteroarylene group having 5 to 30 carbon atoms. As used herein, “atoms for forming a ring” may refer to “ring-forming atoms”. As used herein, “direct linkage” may refer to a bond such as a single bond.

In Formula 1, non-limiting examples of the aryl group having 6 to 30 carbon atoms for forming a ring used as Ar¹ and Ar² may include a phenyl group, a naphthyl group, an anthracenyl group, a phenanthryl group, a biphenyl group, a terphenyl group, a quaterphenyl group, a fluorenyl group, a triphenylene group, a biphenylene group, a pyrenyl group, a benzofluoranthenyl group, a chrysenyl group, a phenylnaphthyl group, a naphthylphenyl group, etc. In some embodiments, the naphthyl group, the biphenyl group, the phenylnaphthyl group and the naphthylphenyl group may be included.

Non-limiting examples of the heteroaryl group having 5 to 30 carbon atoms for forming a ring used as Ar¹ and Ar² may include a pyridyl group, a quinolyl group, an isoquinolyl group, a benzofuryl group, a benzothienyl group, an indolyl group, a benzooxazolyl group, a benzothiazolyl group, a quinoxalyl group, a benzoimidazolyl group, a dibenzofuryl group, a dibenzothienyl group, a carbazolyl group, etc.

In Formula 1, non-limiting examples of the aryl group having 6 to 30 carbon atoms for forming a ring and the heteroaryl group having 5 to 30 carbon atoms for forming a ring used as Ar³ may be the same as those used for Ar¹ and Ar² above.

Non-limiting examples of the alkyl group having 1 to 6 carbon atoms used as Ar³ may include a methyl group, an ethyl group, a propyl group, an isopropyl group, an n-butyl group, an s-butyl group, an isobutyl group, a t-butyl group, an n-pentyl group, an n-hexyl group, a hydroxymethyl group, a 1-hydroxyethyl group, a 2-hydroxyethyl group, a 2-hydroxyisobutyl group, a 1,2-dihydroxyethyl group, a 1,3-dihydroxyisopropyl group, a 2,3-dihydroxy-t-butyl group, a 1,2,3-trihydroxypropyl group, a chloromethyl group, a 1-chloroethyl group, a 2-chloroethyl group, a 2-chloroisobutyl group, a 1,2-dichloroethyl group, a 1,3-dichloroisopropyl group, a 2,3-dichloro-t-butyl group, a 1,2,3-trichloropropyl group, a bromomethyl group, a 1-bromoethyl group, a 2-bromoethyl group, a 2-bromoisobutyl group, a 1,2-dibromoethyl group, a 1,3-dibromoisopropyl group, a 2,3-dibromo-t-butyl group, a 1,2,3-tribromopropyl group, an iodomethyl group, a 1-iodoethyl group, a 2-iodoethyl group, a 2-iodoisobutyl group, a 1,2-diiodoethyl group, a 1,3-diiodoisopropyl group, a 2,3-diiodo-t-butyl group, a 1,2,3-triiodopropyl group, a cyanomethyl group, a 1-cyanoethyl group, a 2-cyanoethyl group, a 2-cyanoisobutyl group, a 1,2-dicyanoethyl group, a 1,3-dicyanoisopropyl group, a 2,3-dicyano-t-butyl group, a 1,2,3-tricyanopropyl group, a nitromethyl group, a 1-nitroethyl group, a 2-nitroethyl group, a 2-nitroisobutyl group, a 1,2-dinitroethyl group, a 1,3-dinitroisopropyl group, a 2,3-dinitro-t-butyl group, a 1,2,3-trinitropropyl group, a cyclopropyl group, a cyclobutyl group, a cyclopentyl group, a cyclohexyl group, a 4-methylcyclohexyl group, etc.

In some embodiments, Ar¹ to Ar³ may be selected from a substituted or unsubstituted aryl group, a substituted or unsubstituted phenyl group, a naphthyl group, a naphthylphenyl group, a phenylnaphthyl group, and a biphenyl group.

In some embodiments, in Formula 1, the heteroaryl group having 5 to 30 carbon atoms for forming a ring used as HAr may be the same as those used for Ar¹ and Ar² above. In some embodiments, HAr may be a substituted or unsubstituted dibenzofuryl group or dibenzothienyl group. As described above, HAr may be a different substituent from Ar³.

In Formula 1, non-limiting examples of the arylene group having 6 to 30 carbon atoms for forming a ring and the heteroarylene group having 5 to 30 carbon atoms for forming a ring used as L may include a phenylene group, a biphenylene group, a terphenylene group, a naphthylene group, an anthrylene group, a phenanthrylene group, a fluorenylene group, an indenylene group, a pyrenylene group, an acetylnaphthenylene group, a fluoranthenyl group, a triphenylenyl group, a pyridylene group, a pyranylene group, a quinolylene group, an isoquinolylene group, a benzofuranylene group, a benzothienylene group, an indolylene group, a carbazolyl group, a benzooxazolylene group, a benzothiazolylene group, a quinoxaline group, a benzoimidazolyl group, a pyrazolylene group, a dibenzofuranylene group, a dibenzothienylene group, etc.

In Formula 1, in the case where Ar¹ to Ar³, HAr, and L have a substituent, the substituent may be selected from an alkyl group (such as a methyl group, an ethyl group, a propyl group, a pentyl group and a hexyl group), and an aryl group (such as a phenyl group, a biphenyl group and a naphthyl group). Ar¹ to Ar³ and HAr may be substituted with a plurality of substituents. A plurality of substituents may combine (e.g., couple) to form a saturated or unsaturated ring.

The amine compound represented by Formula 1 as the material for an organic EL device, according to an embodiment of the present disclosure, may include a heteroaryl group HAr and a substituent Ar³ different from the heteroaryl group at position 9 of a fluorenyl group combined or coupled with the amine moiety, such that the entire molecule is substantially asymmetric, and the material may be more amorphous. Accordingly, charge transportation may become smooth (e.g., may be improved), and the lifetime of the organic EL device may be improved.

The material for an organic EL device represented by Formula 1, according to an embodiment of the present disclosure, may be represented by one selected from the following Compounds 1 to 138, without limitation.

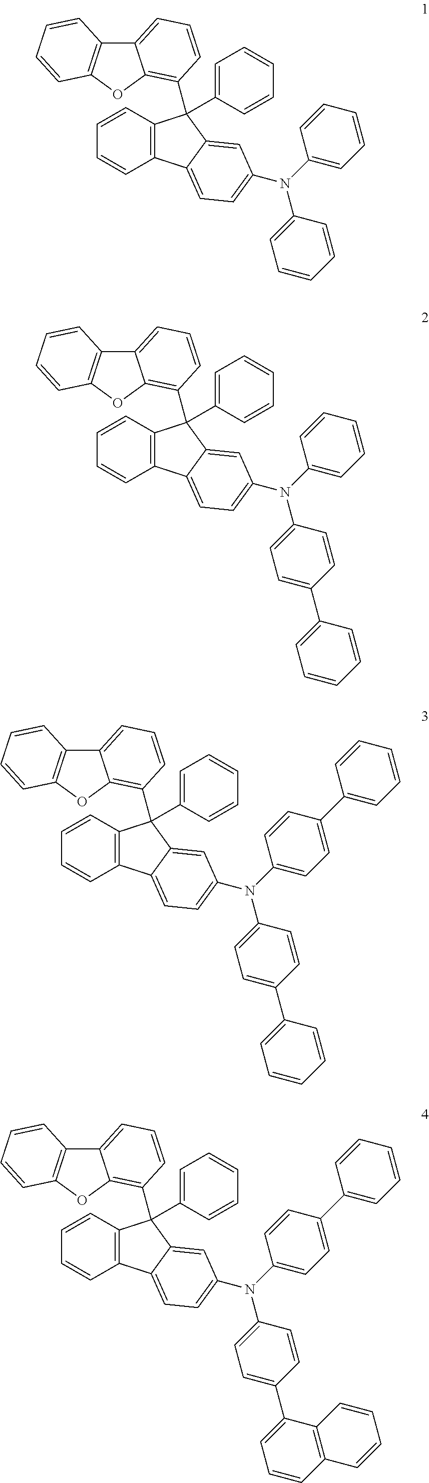 [SMILES: c1ccc(N(c2ccccc2)c2ccc3c(c2)C(c2ccccc2)(c2cccc4c2oc2ccccc24)c2ccccc2-3)cc1 ; c1ccc(-c2ccc(N(c3ccccc3)c3ccc4c(c3)C(c3ccccc3)(c3cccc5c3oc3ccccc35)c3ccccc3-4)cc2)cc1 ; c1ccc(-c2ccc(N(c3ccc(-c4ccccc4)cc3)c3ccc4c(c3)C(c3ccccc3)(c3cccc5c3oc3ccccc35)c3ccccc3-4)cc2)cc1 ; c1ccc(-c2ccc(N(c3ccc(-c4cccc5ccccc45)cc3)c3ccc4c(c3)C(c3ccccc3)(c3cccc5c3oc3ccccc35)c3ccccc3-4)cc2)cc1]

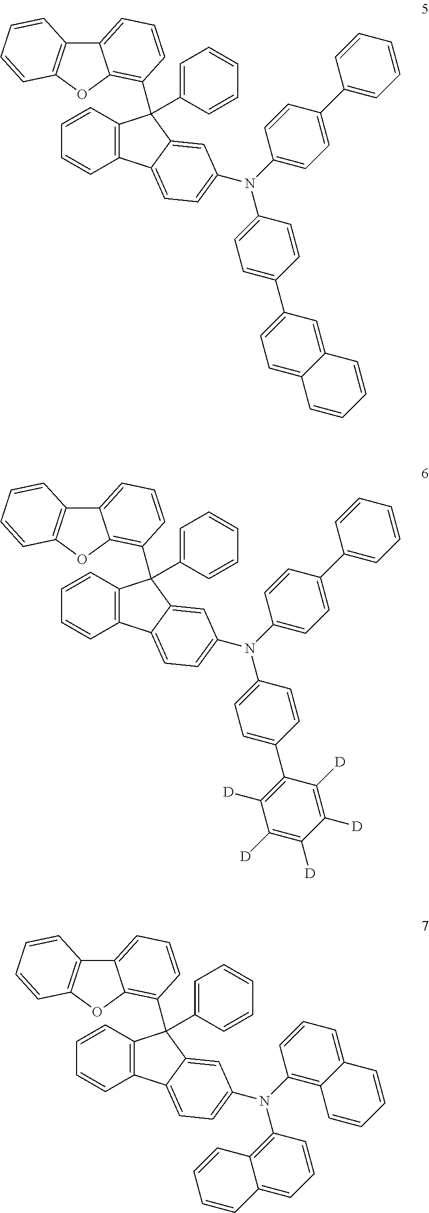 [SMILES: c1ccc(-c2ccc(N(c3ccc(-c4ccc5ccccc5c4)cc3)c3ccc4c(c3)C(c3ccccc3)(c3cccc5c3oc3ccccc35)c3ccccc3-4)cc2)cc1 ; [2H]c1c([2H])c([2H])c(-c2ccc(N(c3ccc(-c4ccccc4)cc3)c3ccc4c(c3)C(c3ccccc3)(c3cccc5c3oc3ccccc35)c3ccccc3-4)cc2)c([2H])c1[2H] ; c1ccc(C2(c3cccc4c3oc3ccccc34)c3ccccc3-c3ccc(N(c4cccc5ccccc45)c4cccc5ccccc45)cc32)cc1]

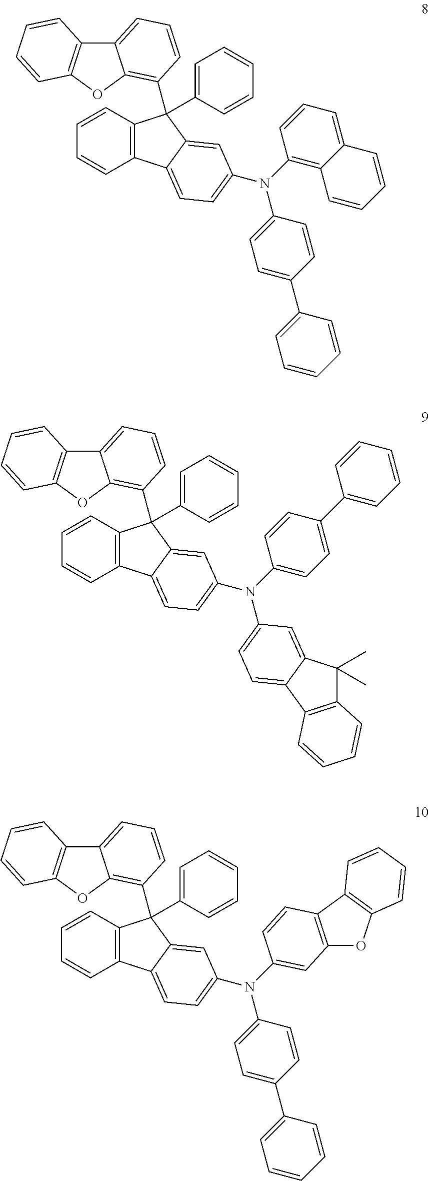 [SMILES: c1ccc(-c2ccc(N(c3ccc4c(c3)C(c3ccccc3)(c3cccc5c3oc3ccccc35)c3ccccc3-4)c3cccc4ccccc34)cc2)cc1 ; CC1(C)c2ccccc2-c2ccc(N(c3ccc(-c4ccccc4)cc3)c3ccc4c(c3)C(c3ccccc3)(c3cccc5c3oc3ccccc35)c3ccccc3-4)cc21 ; c1ccc(-c2ccc(N(c3ccc4c(c3)C(c3ccccc3)(c3cccc5c3oc3ccccc35)c3ccccc3-4)c3ccc4c(c3)oc3ccccc34)cc2)cc1]

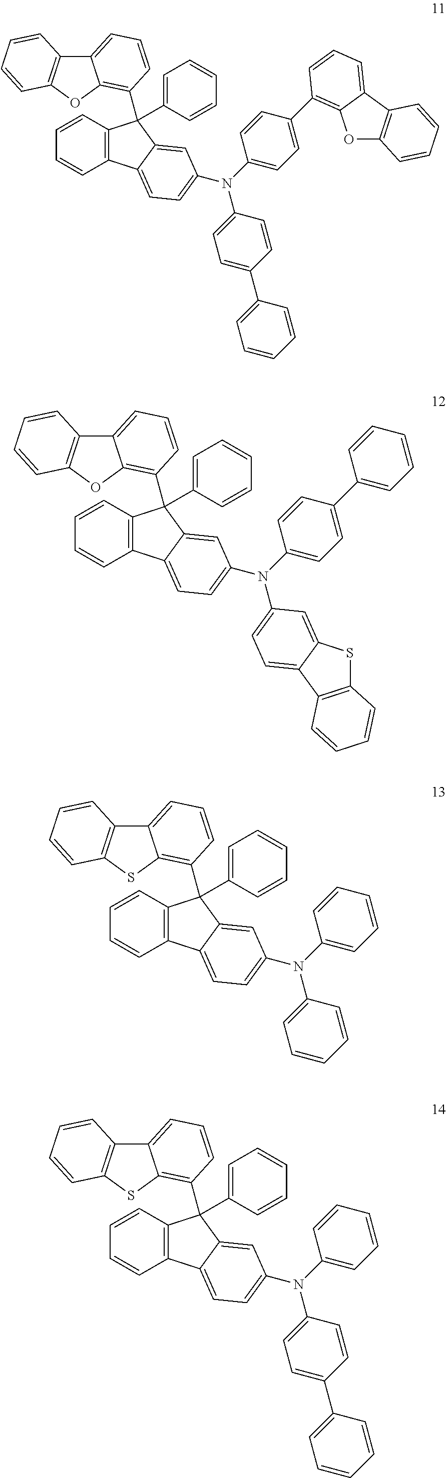 [SMILES: c1ccc(-c2ccc(N(c3ccc(-c4cccc5c4oc4ccccc45)cc3)c3ccc4c(c3)C(c3ccccc3)(c3cccc5c3oc3ccccc35)c3ccccc3-4)cc2)cc1 ; c1ccc(-c2ccc(N(c3ccc4c(c3)C(c3ccccc3)(c3cccc5c3oc3ccccc35)c3ccccc3-4)c3ccc4c(c3)sc3ccccc34)cc2)cc1 ; c1ccc(N(c2ccccc2)c2ccc3c(c2)C(c2ccccc2)(c2cccc4c2sc2ccccc24)c2ccccc2-3)cc1 ; c1ccc(-c2ccc(N(c3ccccc3)c3ccc4c(c3)C(c3ccccc3)(c3cccc5c3sc3ccccc35)c3ccccc3-4)cc2)cc1]

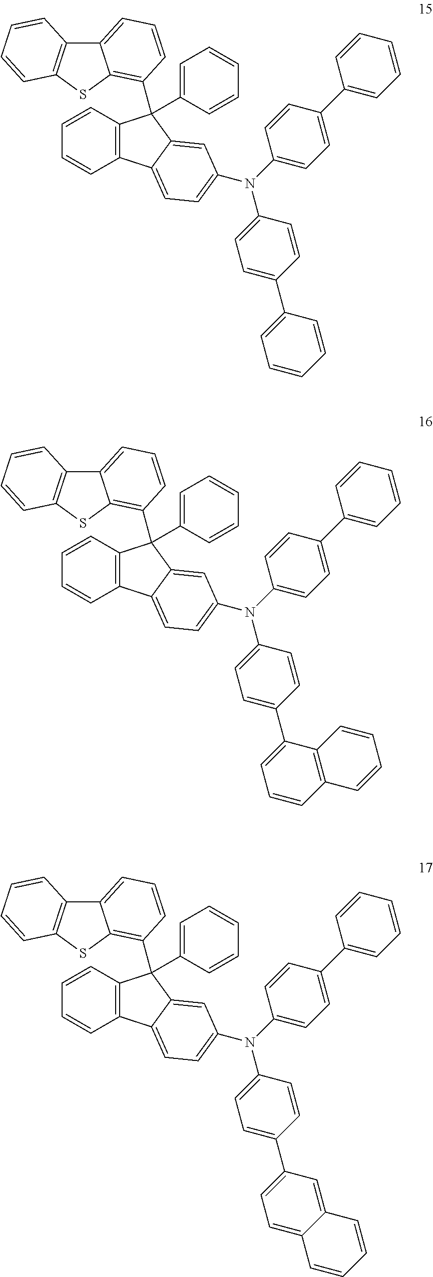 [SMILES: c1ccc(-c2ccc(N(c3ccc(-c4ccccc4)cc3)c3ccc4c(c3)C(c3ccccc3)(c3cccc5c3sc3ccccc35)c3ccccc3-4)cc2)cc1 ; c1ccc(-c2ccc(N(c3ccc(-c4cccc5ccccc45)cc3)c3ccc4c(c3)C(c3ccccc3)(c3cccc5c3sc3ccccc35)c3ccccc3-4)cc2)cc1 ; c1ccc(-c2ccc(N(c3ccc(-c4ccc5ccccc5c4)cc3)c3ccc4c(c3)C(c3ccccc3)(c3cccc5c3sc3ccccc35)c3ccccc3-4)cc2)cc1]

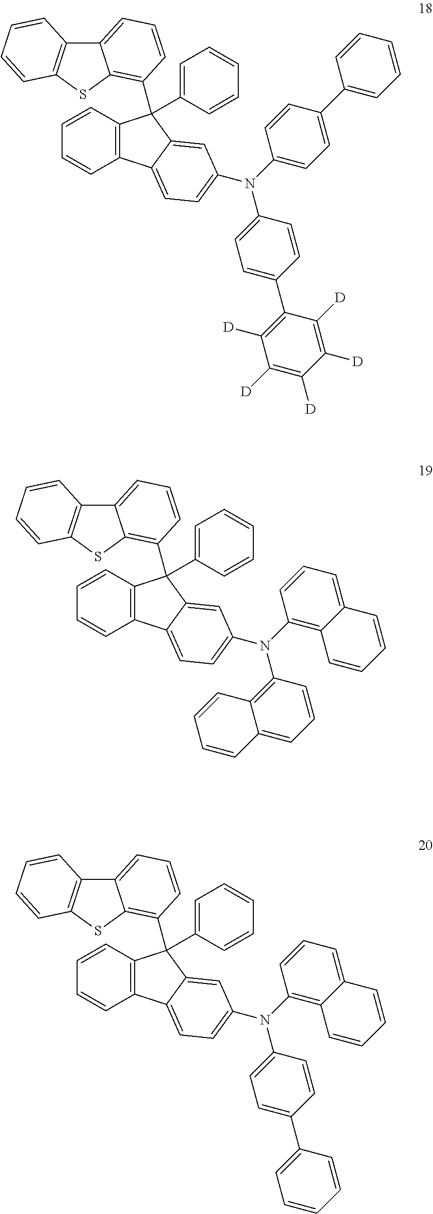 [SMILES: [2H]c1c([2H])c([2H])c(-c2ccc(N(c3ccc(-c4ccccc4)cc3)c3ccc4c(c3)C(c3ccccc3)(c3cccc5c3sc3ccccc35)c3ccccc3-4)cc2)c([2H])c1[2H] ; c1ccc(C2(c3cccc4c3sc3ccccc34)c3ccccc3-c3ccc(N(c4cccc5ccccc45)c4cccc5ccccc45)cc32)cc1 ; c1ccc(-c2ccc(N(c3ccc4c(c3)C(c3ccccc3)(c3cccc5c3sc3ccccc35)c3ccccc3-4)c3cccc4ccccc34)cc2)cc1]

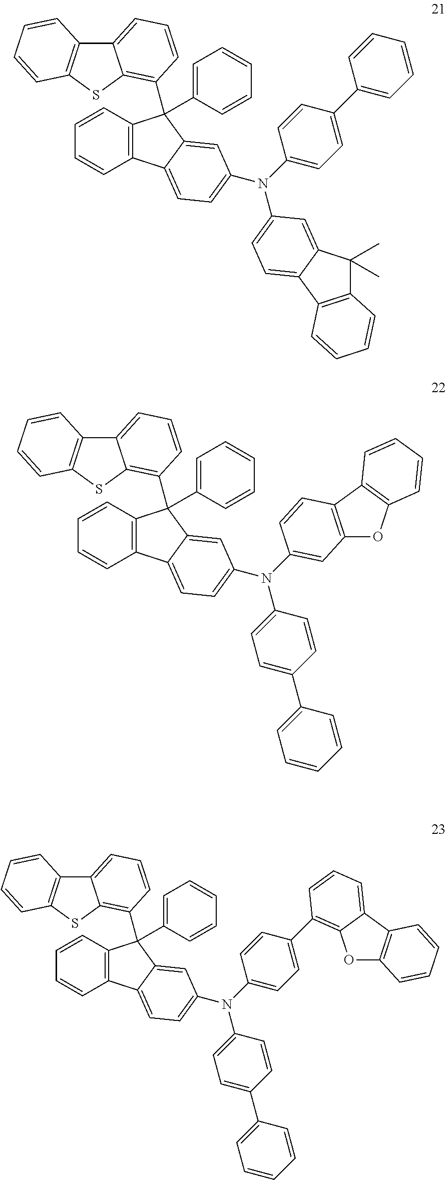 [SMILES: CC1(C)c2ccccc2-c2ccc(N(c3ccc(-c4ccccc4)cc3)c3ccc4c(c3)C(c3ccccc3)(c3cccc5c3sc3ccccc35)c3ccccc3-4)cc21 ; c1ccc(-c2ccc(N(c3ccc4c(c3)C(c3ccccc3)(c3cccc5c3sc3ccccc35)c3ccccc3-4)c3ccc4c(c3)oc3ccccc34)cc2)cc1 ; c1ccc(-c2ccc(N(c3ccc(-c4cccc5c4oc4ccccc45)cc3)c3ccc4c(c3)C(c3ccccc3)(c3cccc5c3sc3ccccc35)c3ccccc3-4)cc2)cc1]

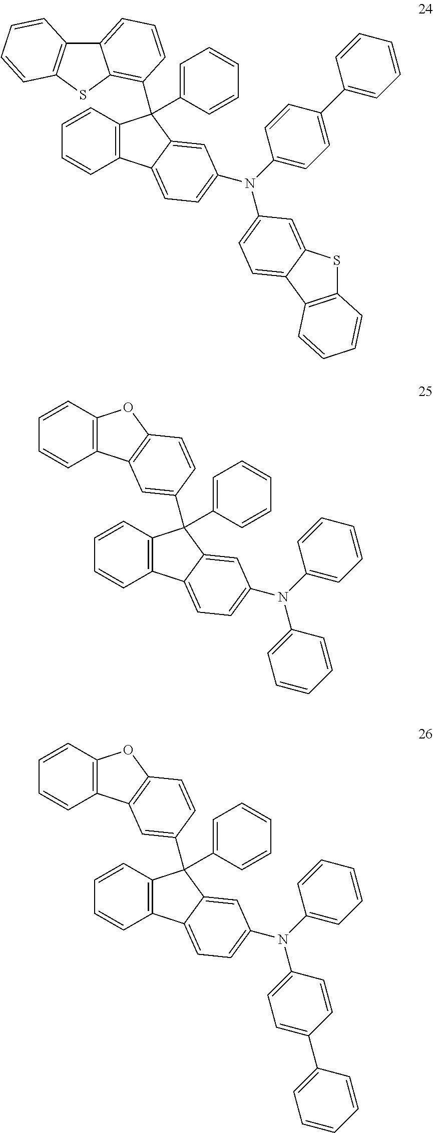 [SMILES: c1ccc(-c2ccc(N(c3ccc4c(c3)C(c3ccccc3)(c3cccc5c3sc3ccccc35)c3ccccc3-4)c3ccc4c(c3)sc3ccccc34)cc2)cc1 ; c1ccc(N(c2ccccc2)c2ccc3c(c2)C(c2ccccc2)(c2ccc4oc5ccccc5c4c2)c2ccccc2-3)cc1 ; c1ccc(-c2ccc(N(c3ccccc3)c3ccc4c(c3)C(c3ccccc3)(c3ccc5oc6ccccc6c5c3)c3ccccc3-4)cc2)cc1]

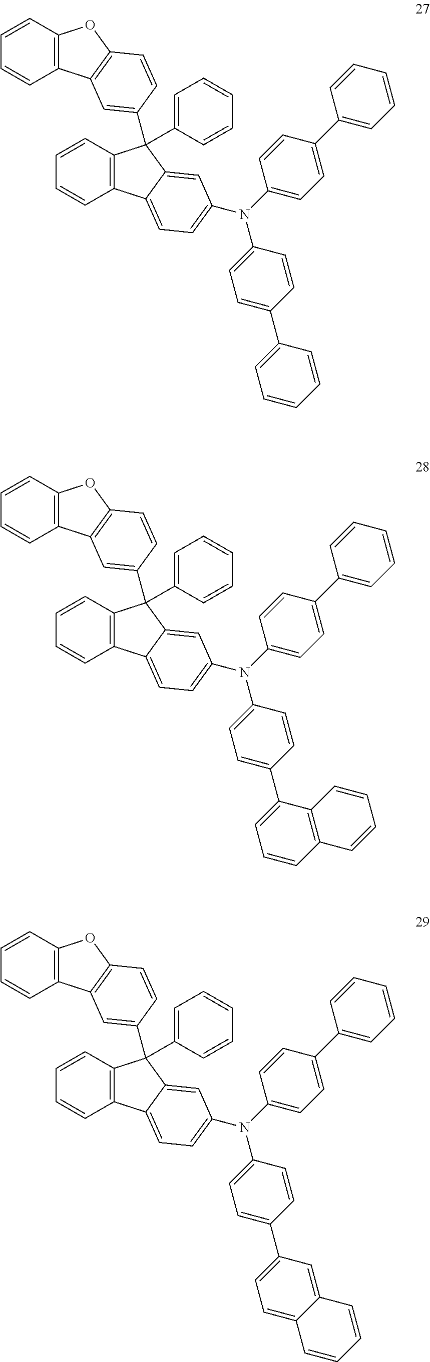 [SMILES: c1ccc(-c2ccc(N(c3ccc(-c4ccccc4)cc3)c3ccc4c(c3)C(c3ccccc3)(c3ccc5oc6ccccc6c5c3)c3ccccc3-4)cc2)cc1 ; c1ccc(-c2ccc(N(c3ccc(-c4cccc5ccccc45)cc3)c3ccc4c(c3)C(c3ccccc3)(c3ccc5oc6ccccc6c5c3)c3ccccc3-4)cc2)cc1 ; c1ccc(-c2ccc(N(c3ccc(-c4ccc5ccccc5c4)cc3)c3ccc4c(c3)C(c3ccccc3)(c3ccc5oc6ccccc6c5c3)c3ccccc3-4)cc2)cc1]

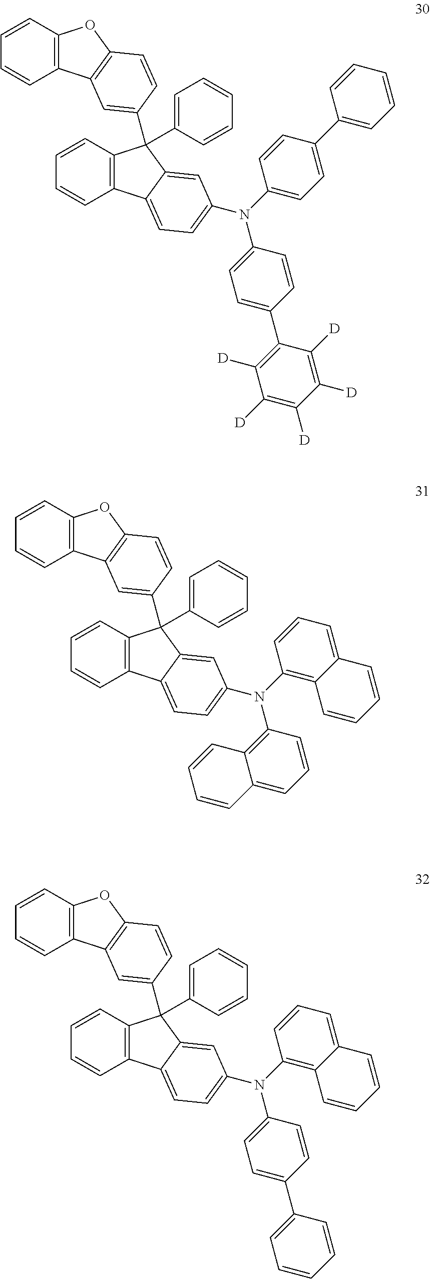 [SMILES: [2H]c1c([2H])c([2H])c(-c2ccc(N(c3ccc(-c4ccccc4)cc3)c3ccc4c(c3)C(c3ccccc3)(c3ccc5oc6ccccc6c5c3)c3ccccc3-4)cc2)c([2H])c1[2H] ; c1ccc(C2(c3ccc4oc5ccccc5c4c3)c3ccccc3-c3ccc(N(c4cccc5ccccc45)c4cccc5ccccc45)cc32)cc1 ; c1ccc(-c2ccc(N(c3ccc4c(c3)C(c3ccccc3)(c3ccc5oc6ccccc6c5c3)c3ccccc3-4)c3cccc4ccccc34)cc2)cc1]

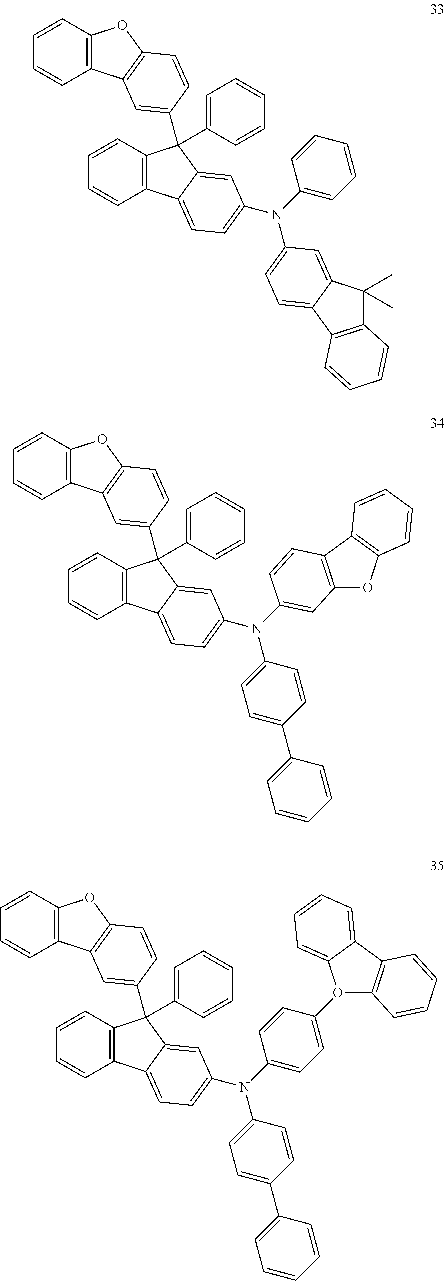

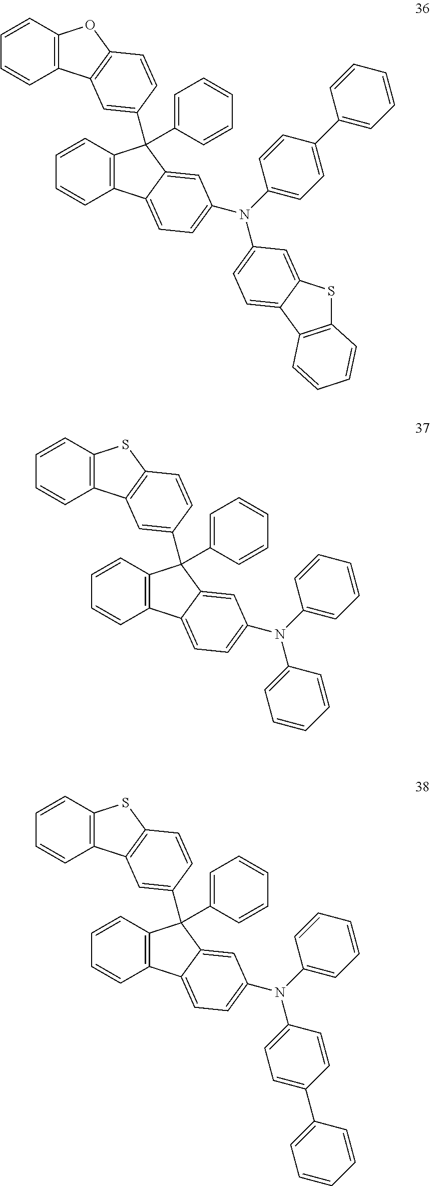 [SMILES: c1ccc(-c2ccc(N(c3ccc4c(c3)C(c3ccccc3)(c3ccc5oc6ccccc6c5c3)c3ccccc3-4)c3ccc4c(c3)sc3ccccc34)cc2)cc1 ; c1ccc(N(c2ccccc2)c2ccc3c(c2)C(c2ccccc2)(c2ccc4sc5ccccc5c4c2)c2ccccc2-3)cc1 ; c1ccc(-c2ccc(N(c3ccccc3)c3ccc4c(c3)C(c3ccccc3)(c3ccc5sc6ccccc6c5c3)c3ccccc3-4)cc2)cc1]

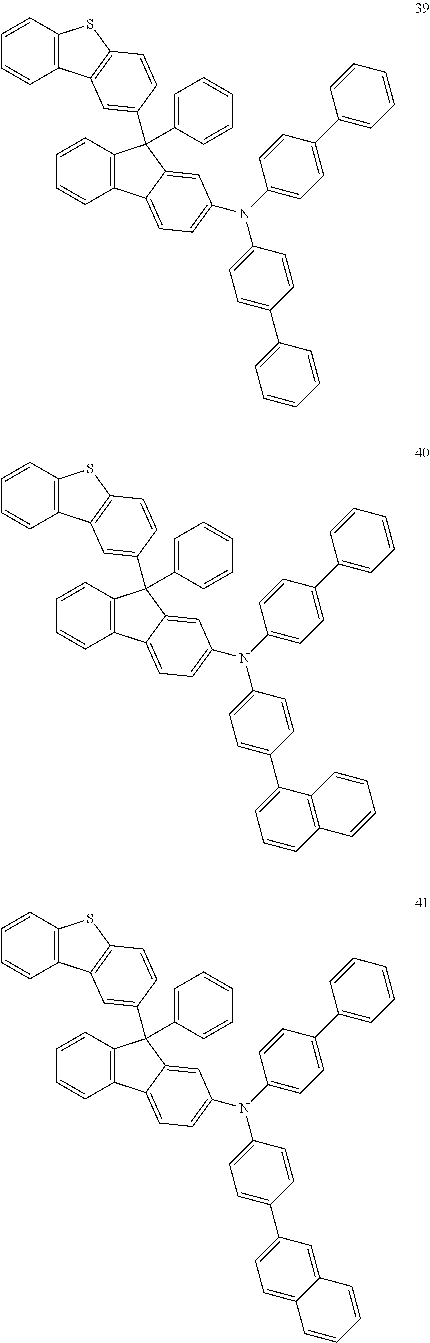 [SMILES: c1ccc(-c2ccc(N(c3ccc(-c4ccccc4)cc3)c3ccc4c(c3)C(c3ccccc3)(c3ccc5sc6ccccc6c5c3)c3ccccc3-4)cc2)cc1 ; c1ccc(-c2ccc(N(c3ccc(-c4cccc5ccccc45)cc3)c3ccc4c(c3)C(c3ccccc3)(c3ccc5sc6ccccc6c5c3)c3ccccc3-4)cc2)cc1 ; c1ccc(-c2ccc(N(c3ccc(-c4ccc5ccccc5c4)cc3)c3ccc4c(c3)C(c3ccccc3)(c3ccc5sc6ccccc6c5c3)c3ccccc3-4)cc2)cc1]

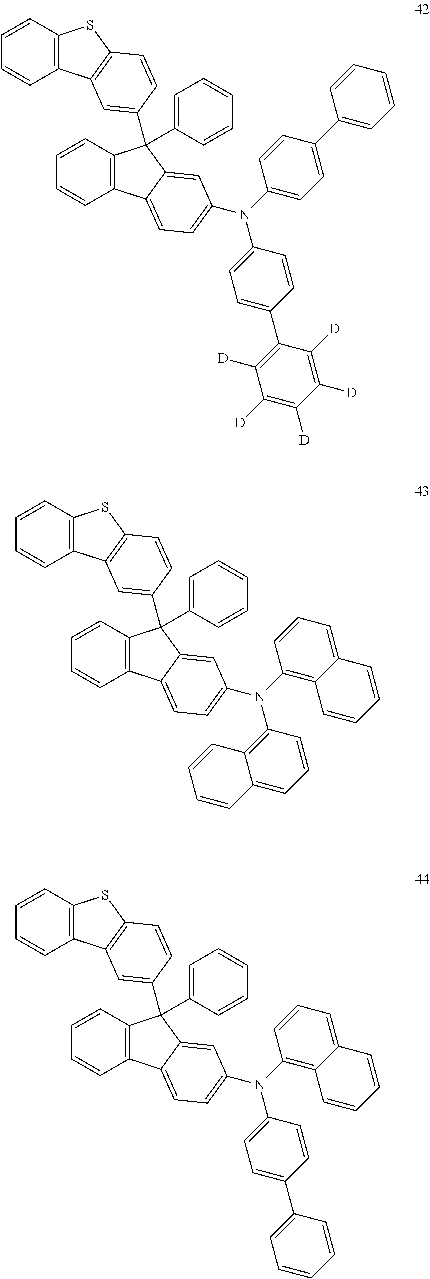 [SMILES: [2H]c1c([2H])c([2H])c(-c2ccc(N(c3ccc(-c4ccccc4)cc3)c3ccc4c(c3)C(c3ccccc3)(c3ccc5sc6ccccc6c5c3)c3ccccc3-4)cc2)c([2H])c1[2H] ; c1ccc(C2(c3ccc4sc5ccccc5c4c3)c3ccccc3-c3ccc(N(c4cccc5ccccc45)c4cccc5ccccc45)cc32)cc1 ; c1ccc(-c2ccc(N(c3ccc4c(c3)C(c3ccccc3)(c3ccc5sc6ccccc6c5c3)c3ccccc3-4)c3cccc4ccccc34)cc2)cc1]

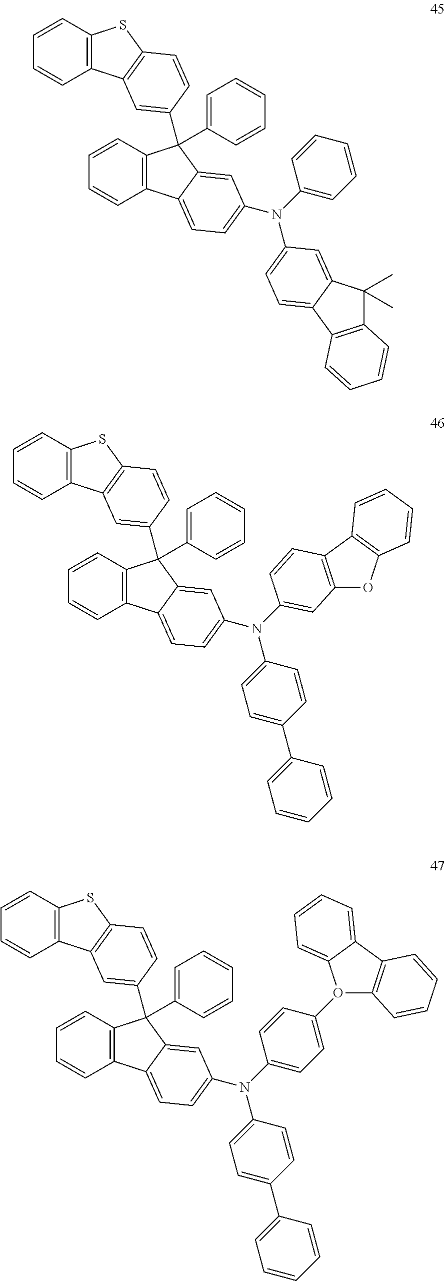

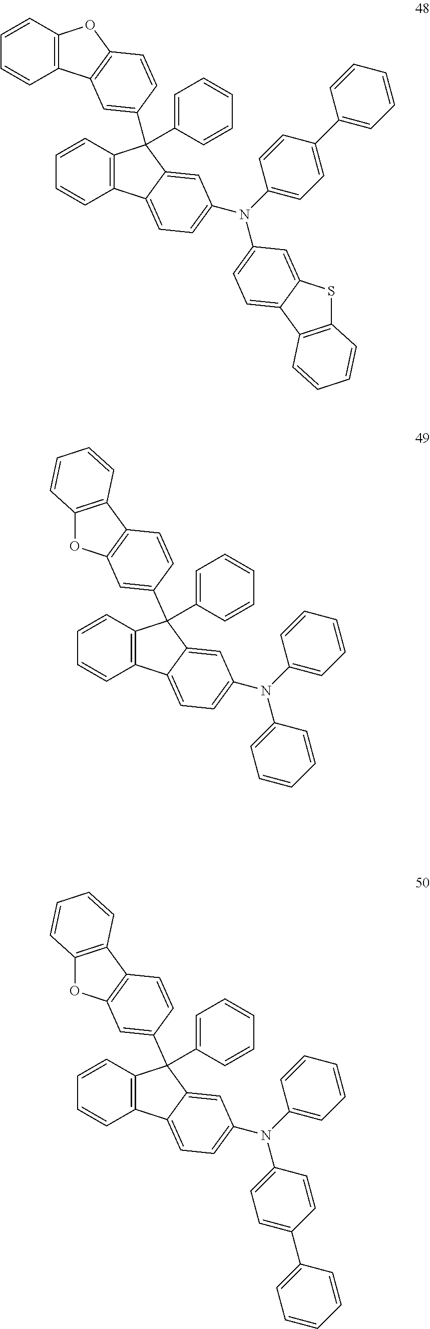 [SMILES: c1ccc(-c2ccc(N(c3ccc4c(c3)C(c3ccccc3)(c3ccc5oc6ccccc6c5c3)c3ccccc3-4)c3ccc4c(c3)sc3ccccc34)cc2)cc1 ; c1ccc(N(c2ccccc2)c2ccc3c(c2)C(c2ccccc2)(c2ccc4c(c2)oc2ccccc24)c2ccccc2-3)cc1 ; c1ccc(-c2ccc(N(c3ccccc3)c3ccc4c(c3)C(c3ccccc3)(c3ccc5c(c3)oc3ccccc35)c3ccccc3-4)cc2)cc1]

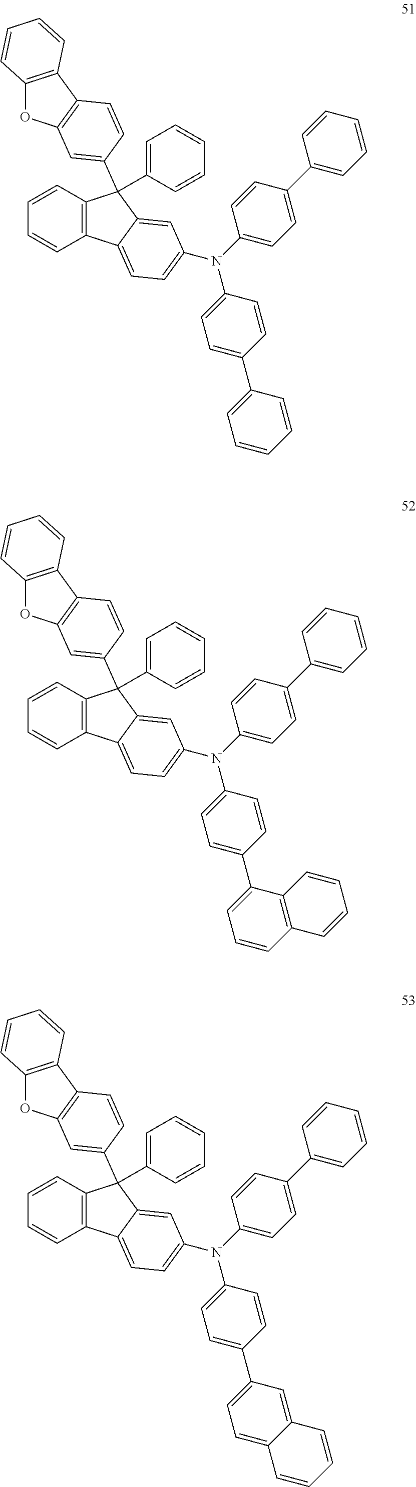 [SMILES: c1ccc(-c2ccc(N(c3ccc(-c4ccccc4)cc3)c3ccc4c(c3)C(c3ccccc3)(c3ccc5c(c3)oc3ccccc35)c3ccccc3-4)cc2)cc1 ; c1ccc(-c2ccc(N(c3ccc(-c4cccc5ccccc45)cc3)c3ccc4c(c3)C(c3ccccc3)(c3ccc5c(c3)oc3ccccc35)c3ccccc3-4)cc2)cc1 ; c1ccc(-c2ccc(N(c3ccc(-c4ccc5ccccc5c4)cc3)c3ccc4c(c3)C(c3ccccc3)(c3ccc5c(c3)oc3ccccc35)c3ccccc3-4)cc2)cc1]

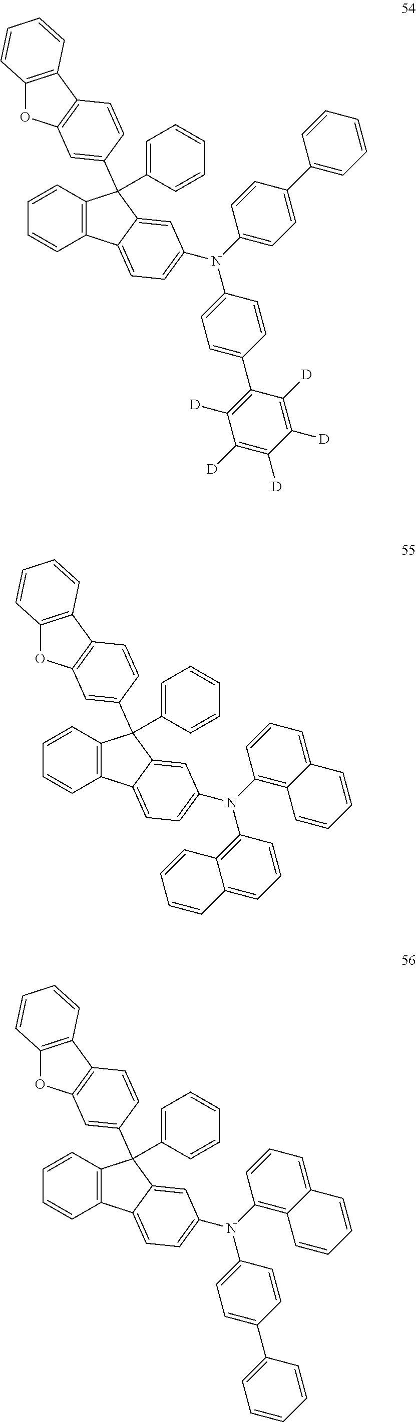 [SMILES: [2H]c1c([2H])c([2H])c(-c2ccc(N(c3ccc(-c4ccccc4)cc3)c3ccc4c(c3)C(c3ccccc3)(c3ccc5c(c3)oc3ccccc35)c3ccccc3-4)cc2)c([2H])c1[2H] ; c1ccc(C2(c3ccc4c(c3)oc3ccccc34)c3ccccc3-c3ccc(N(c4cccc5ccccc45)c4cccc5ccccc45)cc32)cc1 ; c1ccc(-c2ccc(N(c3ccc4c(c3)C(c3ccccc3)(c3ccc5c(c3)oc3ccccc35)c3ccccc3-4)c3cccc4ccccc34)cc2)cc1]

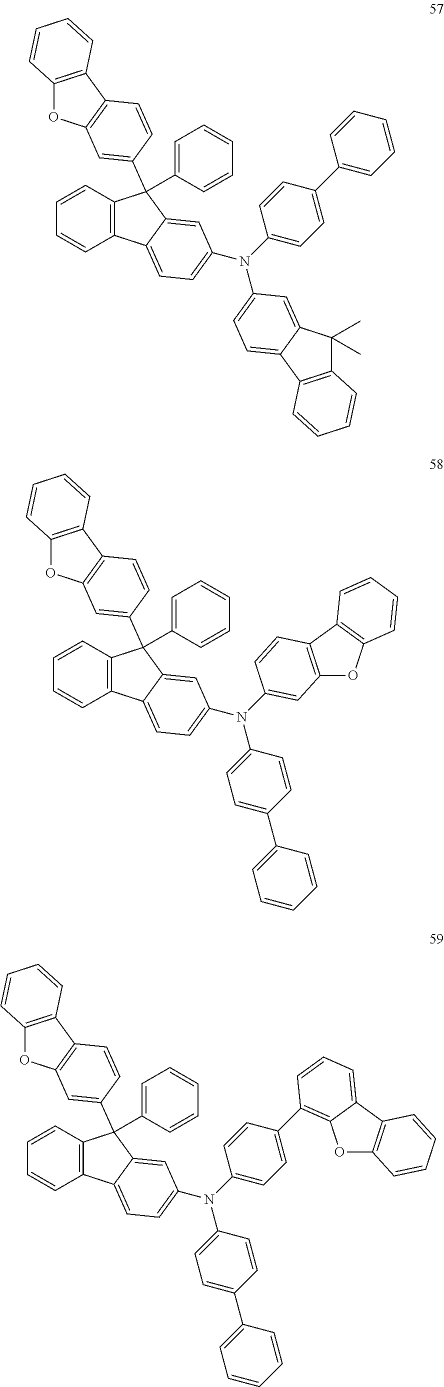 [SMILES: CC1(C)c2ccccc2-c2ccc(N(c3ccc(-c4ccccc4)cc3)c3ccc4c(c3)C(c3ccccc3)(c3ccc5c(c3)oc3ccccc35)c3ccccc3-4)cc21 ; c1ccc(-c2ccc(N(c3ccc4c(c3)C(c3ccccc3)(c3ccc5c(c3)oc3ccccc35)c3ccccc3-4)c3ccc4c(c3)oc3ccccc34)cc2)cc1 ; c1ccc(-c2ccc(N(c3ccc(-c4cccc5c4oc4ccccc45)cc3)c3ccc4c(c3)C(c3ccccc3)(c3ccc5c(c3)oc3ccccc35)c3ccccc3-4)cc2)cc1]

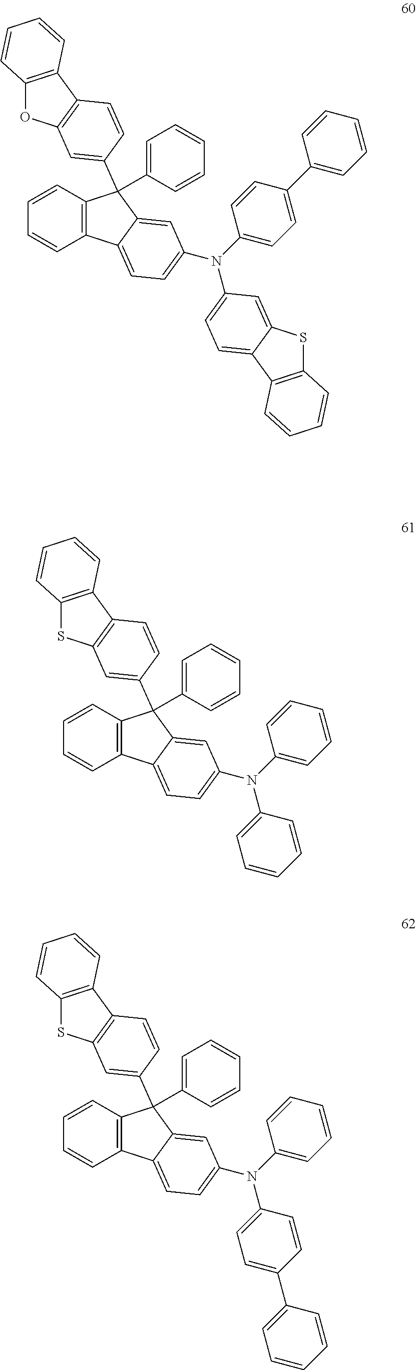 [SMILES: c1ccc(-c2ccc(N(c3ccc4c(c3)C(c3ccccc3)(c3ccc5c(c3)oc3ccccc35)c3ccccc3-4)c3ccc4c(c3)sc3ccccc34)cc2)cc1 ; c1ccc(N(c2ccccc2)c2ccc3c(c2)C(c2ccccc2)(c2ccc4c(c2)sc2ccccc24)c2ccccc2-3)cc1 ; c1ccc(-c2ccc(N(c3ccccc3)c3ccc4c(c3)C(c3ccccc3)(c3ccc5c(c3)sc3ccccc35)c3ccccc3-4)cc2)cc1]

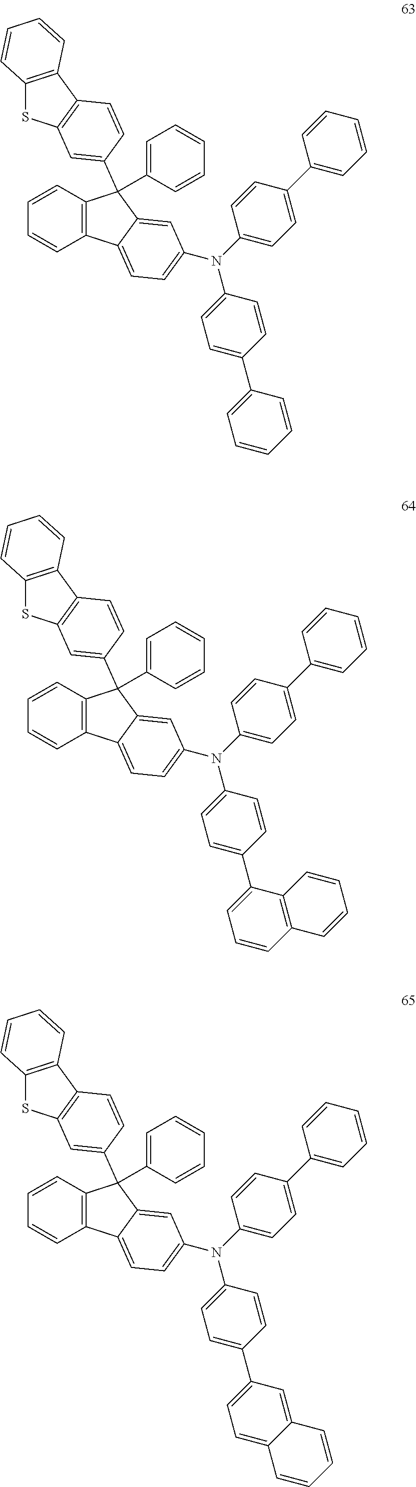 [SMILES: c1ccc(-c2ccc(N(c3ccc(-c4ccccc4)cc3)c3ccc4c(c3)C(c3ccccc3)(c3ccc5c(c3)sc3ccccc35)c3ccccc3-4)cc2)cc1 ; c1ccc(-c2ccc(N(c3ccc(-c4cccc5ccccc45)cc3)c3ccc4c(c3)C(c3ccccc3)(c3ccc5c(c3)sc3ccccc35)c3ccccc3-4)cc2)cc1 ; c1ccc(-c2ccc(N(c3ccc(-c4ccc5ccccc5c4)cc3)c3ccc4c(c3)C(c3ccccc3)(c3ccc5c(c3)sc3ccccc35)c3ccccc3-4)cc2)cc1]

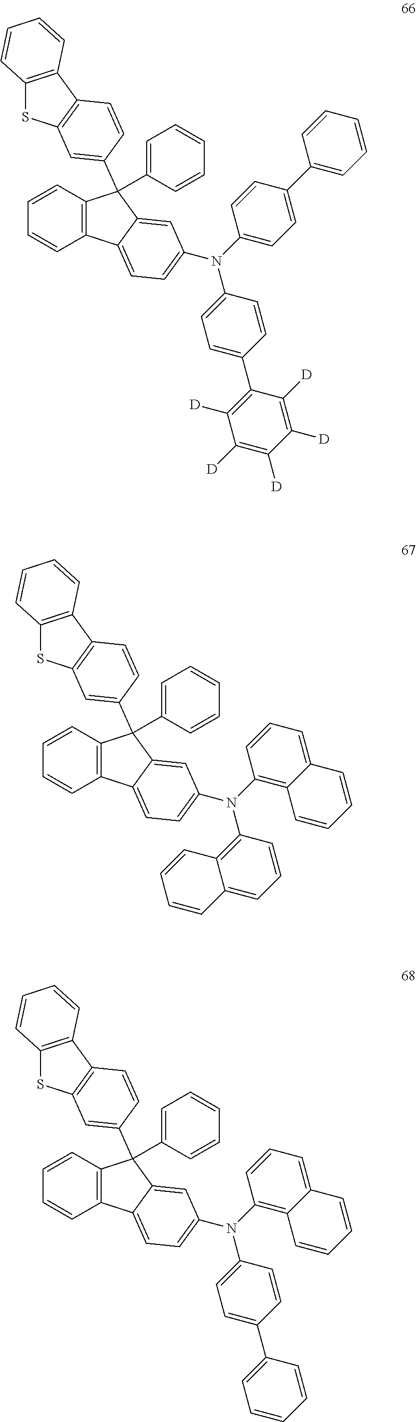 [SMILES: [2H]c1c([2H])c([2H])c(-c2ccc(N(c3ccc(-c4ccccc4)cc3)c3ccc4c(c3)C(c3ccccc3)(c3ccc5c(c3)sc3ccccc35)c3ccccc3-4)cc2)c([2H])c1[2H] ; c1ccc(C2(c3ccc4c(c3)sc3ccccc34)c3ccccc3-c3ccc(N(c4cccc5ccccc45)c4cccc5ccccc45)cc32)cc1 ; c1ccc(-c2ccc(N(c3ccc4c(c3)C(c3ccccc3)(c3ccc5c(c3)sc3ccccc35)c3ccccc3-4)c3cccc4ccccc34)cc2)cc1]

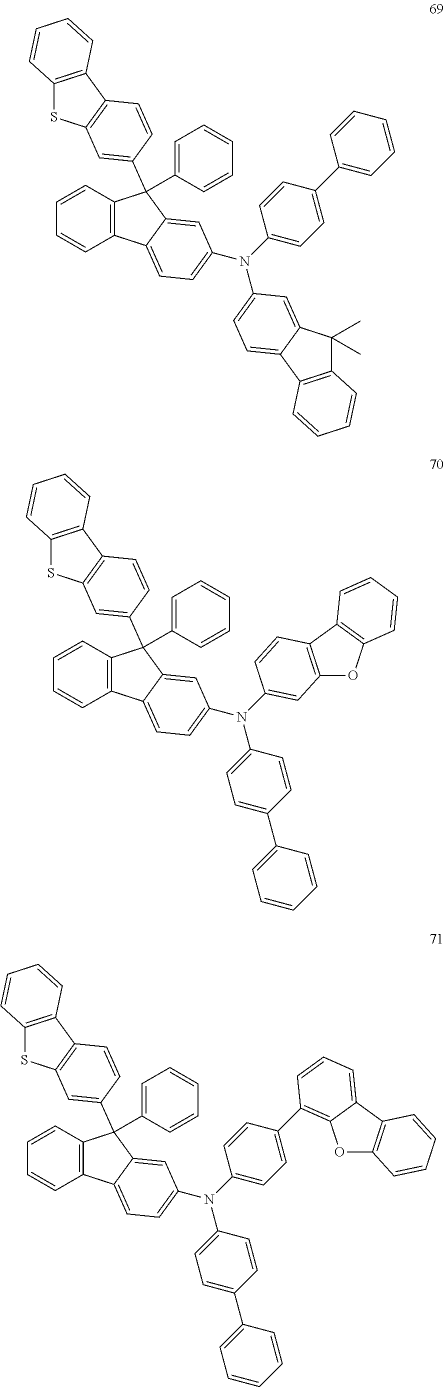 [SMILES: CC1(C)c2ccccc2-c2ccc(N(c3ccc(-c4ccccc4)cc3)c3ccc4c(c3)C(c3ccccc3)(c3ccc5c(c3)sc3ccccc35)c3ccccc3-4)cc21 ; c1ccc(-c2ccc(N(c3ccc4c(c3)C(c3ccccc3)(c3ccc5c(c3)sc3ccccc35)c3ccccc3-4)c3ccc4c(c3)oc3ccccc34)cc2)cc1 ; c1ccc(-c2ccc(N(c3ccc(-c4cccc5c4oc4ccccc45)cc3)c3ccc4c(c3)C(c3ccccc3)(c3ccc5c(c3)sc3ccccc35)c3ccccc3-4)cc2)cc1]

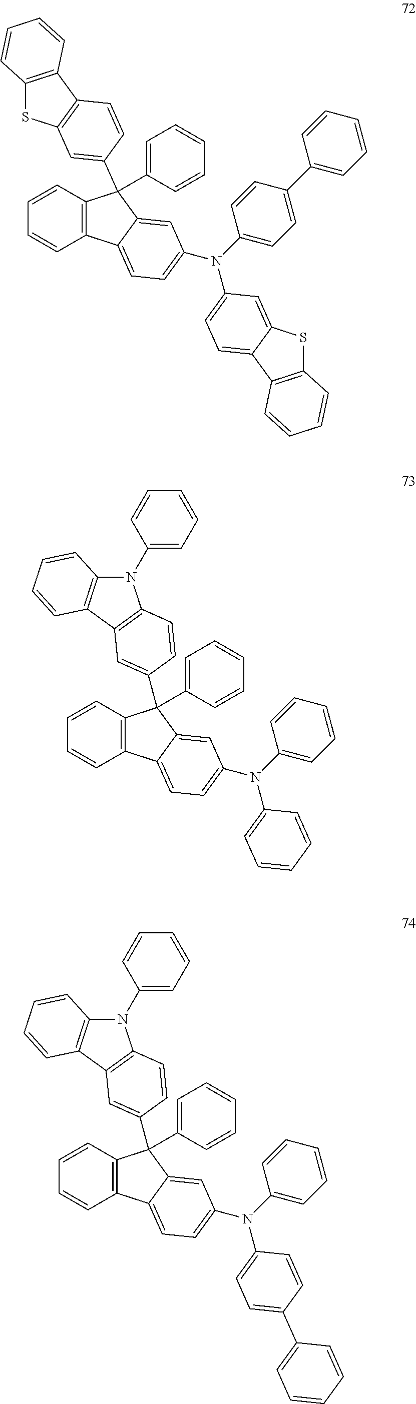 [SMILES: c1ccc(-c2ccc(N(c3ccc4c(c3)C(c3ccccc3)(c3ccc5c(c3)sc3ccccc35)c3ccccc3-4)c3ccc4c(c3)sc3ccccc34)cc2)cc1 ; c1ccc(N(c2ccccc2)c2ccc3c(c2)C(c2ccccc2)(c2ccc4c(c2)c2ccccc2n4-c2ccccc2)c2ccccc2-3)cc1 ; c1ccc(-c2ccc(N(c3ccccc3)c3ccc4c(c3)C(c3ccccc3)(c3ccc5c(c3)c3ccccc3n5-c3ccccc3)c3ccccc3-4)cc2)cc1]

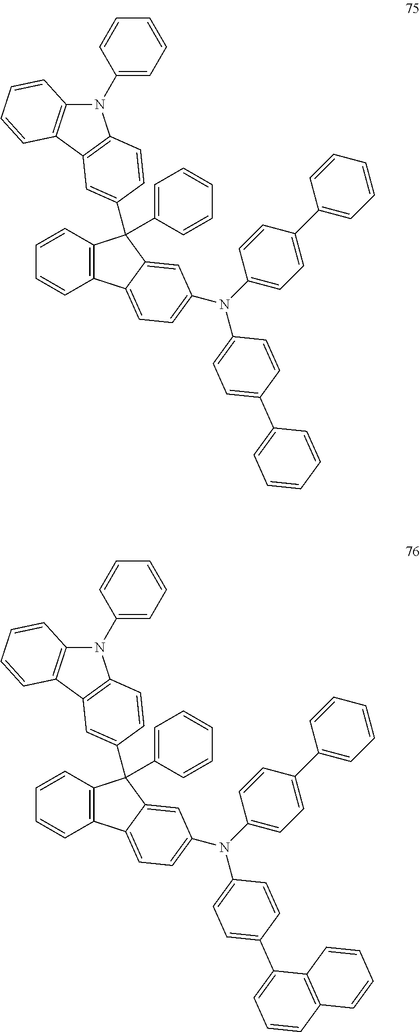 [SMILES: c1ccc(-c2ccc(N(c3ccc(-c4ccccc4)cc3)c3ccc4c(c3)C(c3ccccc3)(c3ccc5c(c3)c3ccccc3n5-c3ccccc3)c3ccccc3-4)cc2)cc1 ; c1ccc(-c2ccc(N(c3ccc(-c4cccc5ccccc45)cc3)c3ccc4c(c3)C(c3ccccc3)(c3ccc5c(c3)c3ccccc3n5-c3ccccc3)c3ccccc3-4)cc2)cc1]

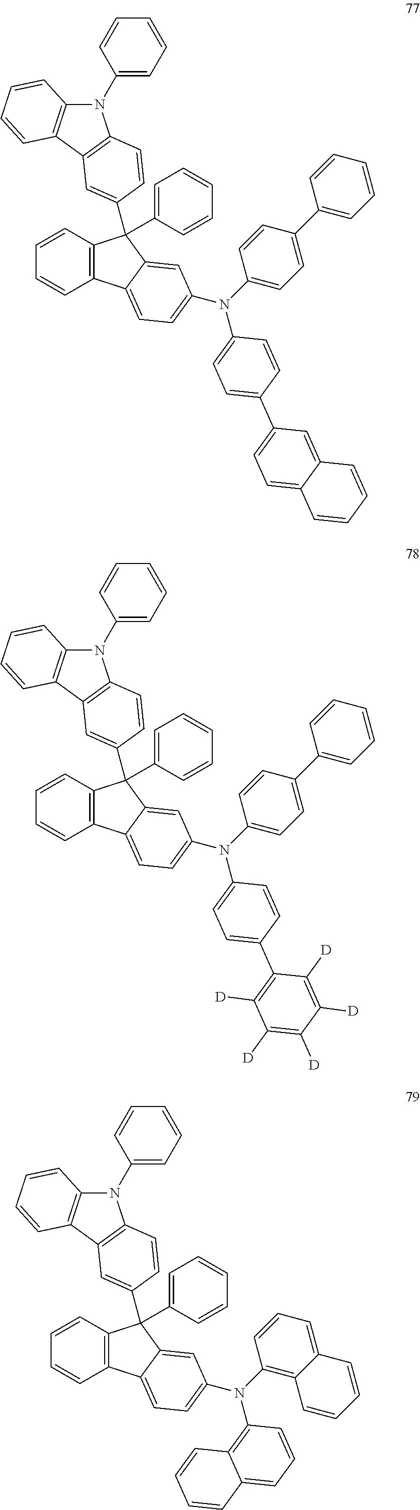 [SMILES: c1ccc(-c2ccc(N(c3ccc(-c4ccc5ccccc5c4)cc3)c3ccc4c(c3)C(c3ccccc3)(c3ccc5c(c3)c3ccccc3n5-c3ccccc3)c3ccccc3-4)cc2)cc1 ; [2H]c1c([2H])c([2H])c(-c2ccc(N(c3ccc(-c4ccccc4)cc3)c3ccc4c(c3)C(c3ccccc3)(c3ccc5c(c3)c3ccccc3n5-c3ccccc3)c3ccccc3-4)cc2)c([2H])c1[2H] ; c1ccc(-n2c3ccccc3c3cc(C4(c5ccccc5)c5ccccc5-c5ccc(N(c6cccc7ccccc67)c6cccc7ccccc67)cc54)ccc32)cc1]

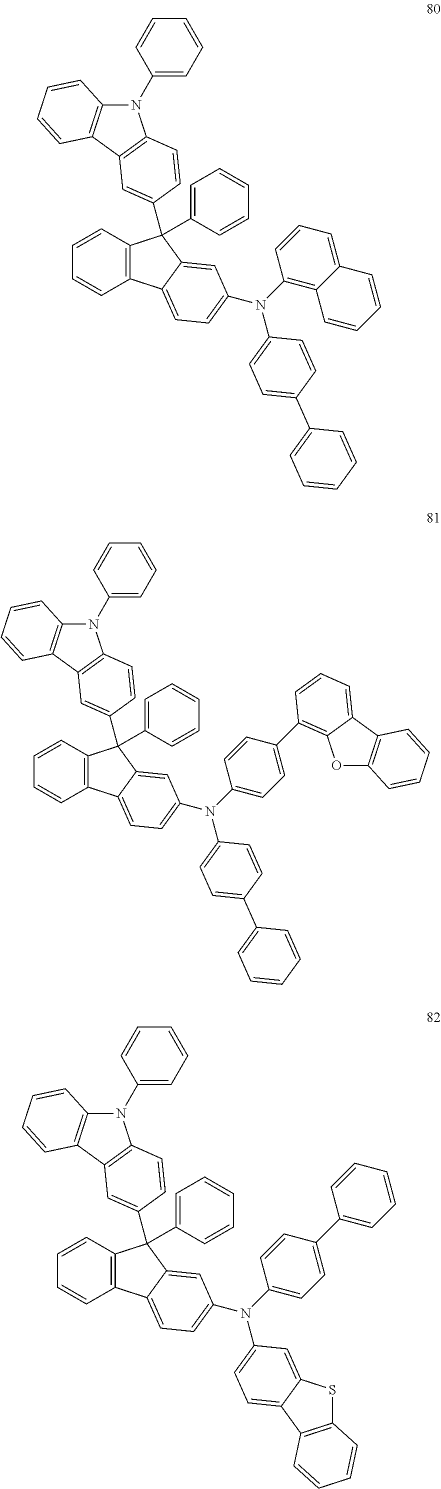 [SMILES: c1ccc(-c2ccc(N(c3ccc4c(c3)C(c3ccccc3)(c3ccc5c(c3)c3ccccc3n5-c3ccccc3)c3ccccc3-4)c3cccc4ccccc34)cc2)cc1 ; c1ccc(-c2ccc(N(c3ccc(-c4cccc5c4oc4ccccc45)cc3)c3ccc4c(c3)C(c3ccccc3)(c3ccc5c(c3)c3ccccc3n5-c3ccccc3)c3ccccc3-4)cc2)cc1 ; c1ccc(-c2ccc(N(c3ccc4c(c3)C(c3ccccc3)(c3ccc5c(c3)c3ccccc3n5-c3ccccc3)c3ccccc3-4)c3ccc4c(c3)sc3ccccc34)cc2)cc1]

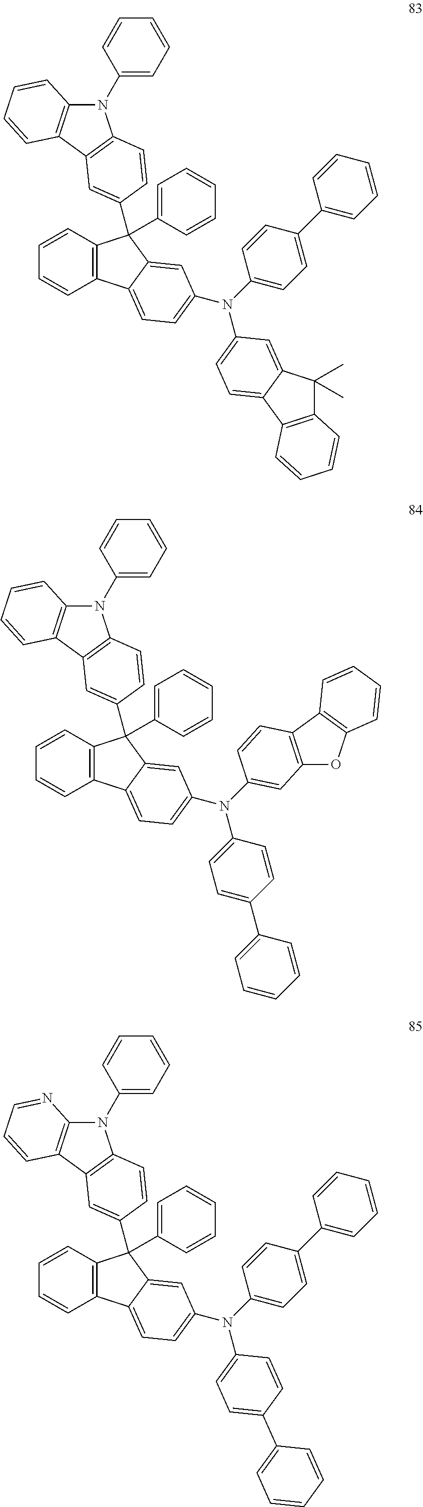 [SMILES: CC1(C)c2ccccc2-c2ccc(N(c3ccc(-c4ccccc4)cc3)c3ccc4c(c3)C(c3ccccc3)(c3ccc5c(c3)c3ccccc3n5-c3ccccc3)c3ccccc3-4)cc21 ; c1ccc(-c2ccc(N(c3ccc4c(c3)C(c3ccccc3)(c3ccc5c(c3)c3ccccc3n5-c3ccccc3)c3ccccc3-4)c3ccc4c(c3)oc3ccccc34)cc2)cc1 ; c1ccc(-c2ccc(N(c3ccc(-c4ccccc4)cc3)c3ccc4c(c3)C(c3ccccc3)(c3ccc5c(c3)c3cccnc3n5-c3ccccc3)c3ccccc3-4)cc2)cc1]

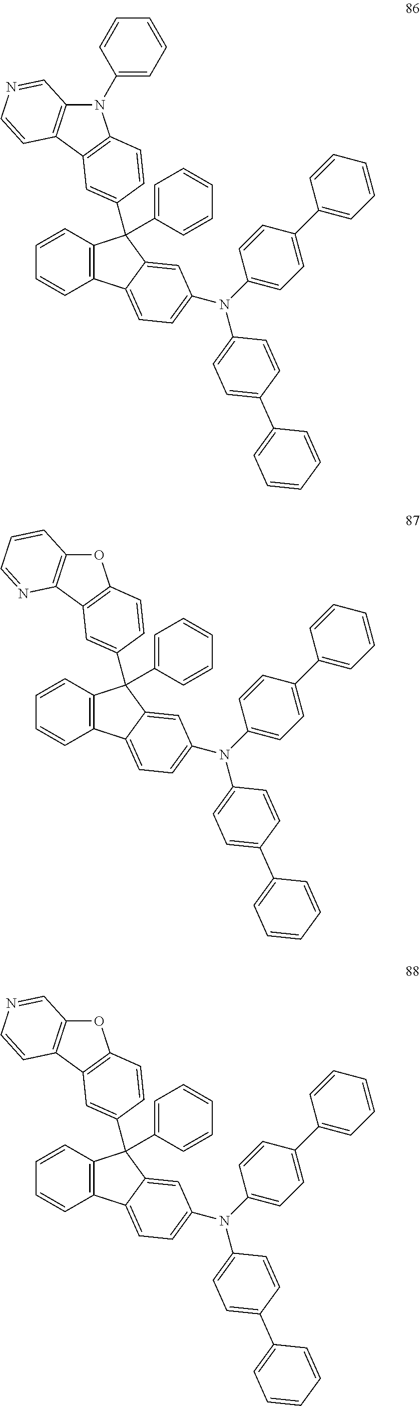 [SMILES: c1ccc(-c2ccc(N(c3ccc(-c4ccccc4)cc3)c3ccc4c(c3)C(c3ccccc3)(c3ccc5c(c3)c3ccncc3n5-c3ccccc3)c3ccccc3-4)cc2)cc1 ; c1ccc(-c2ccc(N(c3ccc(-c4ccccc4)cc3)c3ccc4c(c3)C(c3ccccc3)(c3ccc5oc6cccnc6c5c3)c3ccccc3-4)cc2)cc1 ; c1ccc(-c2ccc(N(c3ccc(-c4ccccc4)cc3)c3ccc4c(c3)C(c3ccccc3)(c3ccc5oc6cnccc6c5c3)c3ccccc3-4)cc2)cc1]

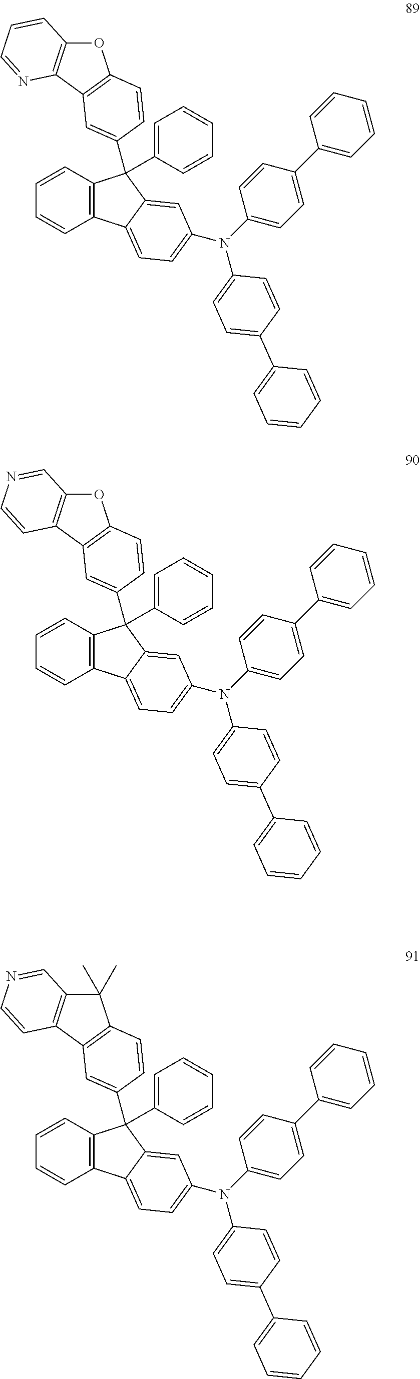 [SMILES: c1ccc(-c2ccc(N(c3ccc(-c4ccccc4)cc3)c3ccc4c(c3)C(c3ccccc3)(c3ccc5oc6cccnc6c5c3)c3ccccc3-4)cc2)cc1 ; c1ccc(-c2ccc(N(c3ccc(-c4ccccc4)cc3)c3ccc4c(c3)C(c3ccccc3)(c3ccc5oc6cnccc6c5c3)c3ccccc3-4)cc2)cc1 ; CC1(C)c2ccc(C3(c4ccccc4)c4ccccc4-c4ccc(N(c5ccc(-c6ccccc6)cc5)c5ccc(-c6ccccc6)cc5)cc43)cc2-c2ccncc21]

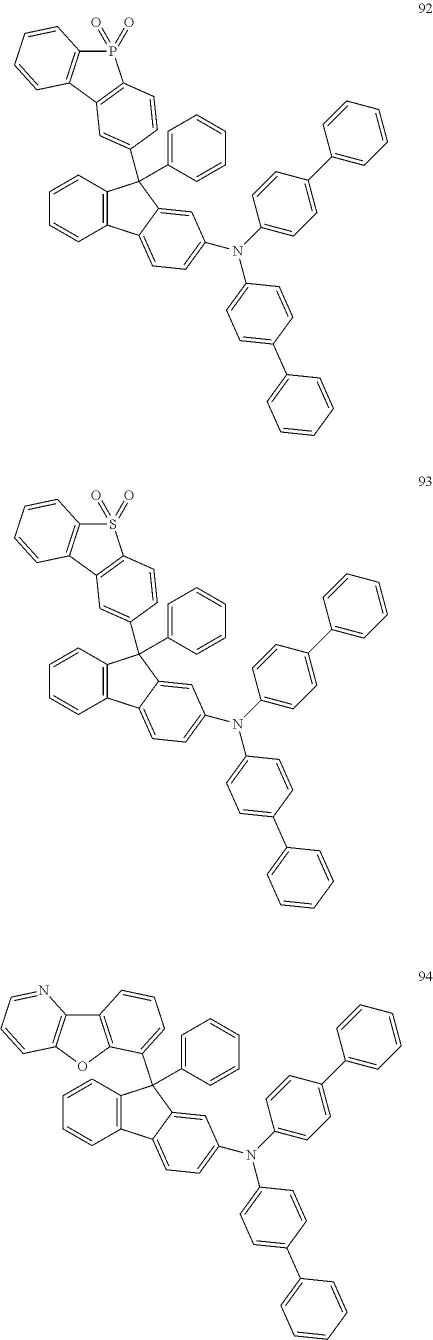

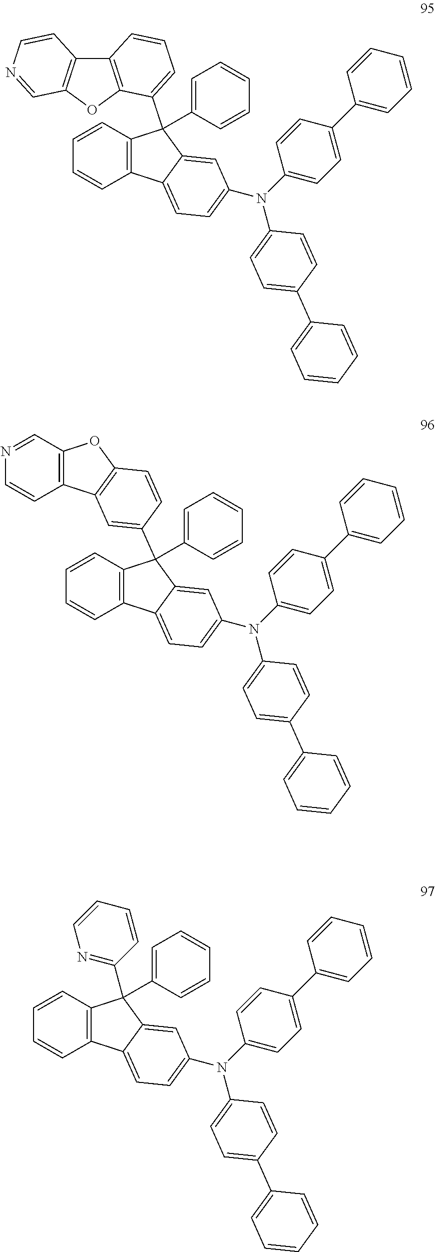 [SMILES: c1ccc(-c2ccc(N(c3ccc(-c4ccccc4)cc3)c3ccc4c(c3)C(c3ccccc3)(c3cccc5c3oc3cnccc35)c3ccccc3-4)cc2)cc1 ; c1ccc(-c2ccc(N(c3ccc(-c4ccccc4)cc3)c3ccc4c(c3)C(c3ccccc3)(c3ccc5oc6cnccc6c5c3)c3ccccc3-4)cc2)cc1 ; c1ccc(-c2ccc(N(c3ccc(-c4ccccc4)cc3)c3ccc4c(c3)C(c3ccccc3)(c3ccccn3)c3ccccc3-4)cc2)cc1]

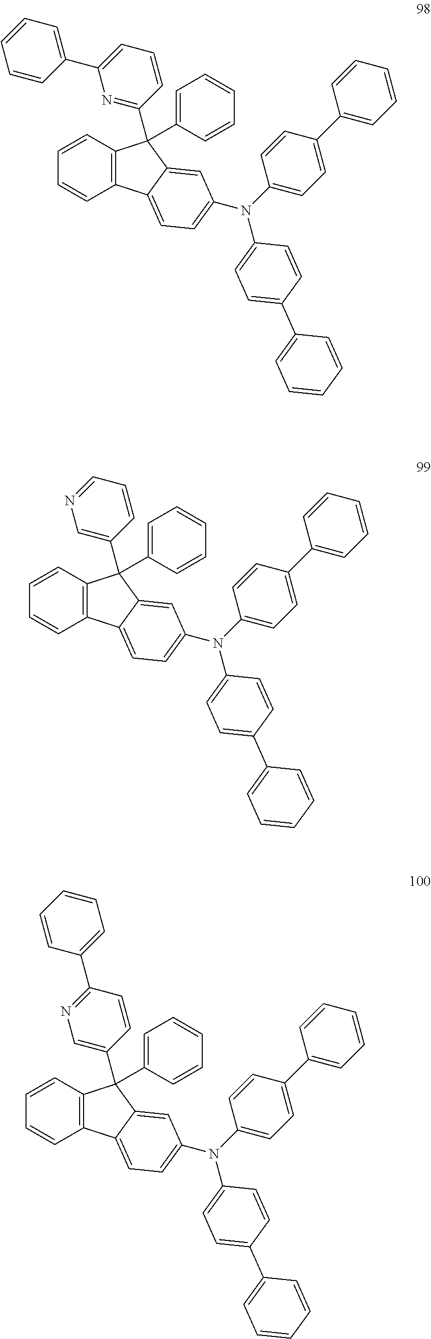 [SMILES: c1ccc(-c2ccc(N(c3ccc(-c4ccccc4)cc3)c3ccc4c(c3)C(c3ccccc3)(c3cccc(-c5ccccc5)n3)c3ccccc3-4)cc2)cc1 ; c1ccc(-c2ccc(N(c3ccc(-c4ccccc4)cc3)c3ccc4c(c3)C(c3ccccc3)(c3cccnc3)c3ccccc3-4)cc2)cc1 ; c1ccc(-c2ccc(N(c3ccc(-c4ccccc4)cc3)c3ccc4c(c3)C(c3ccccc3)(c3ccc(-c5ccccc5)nc3)c3ccccc3-4)cc2)cc1]

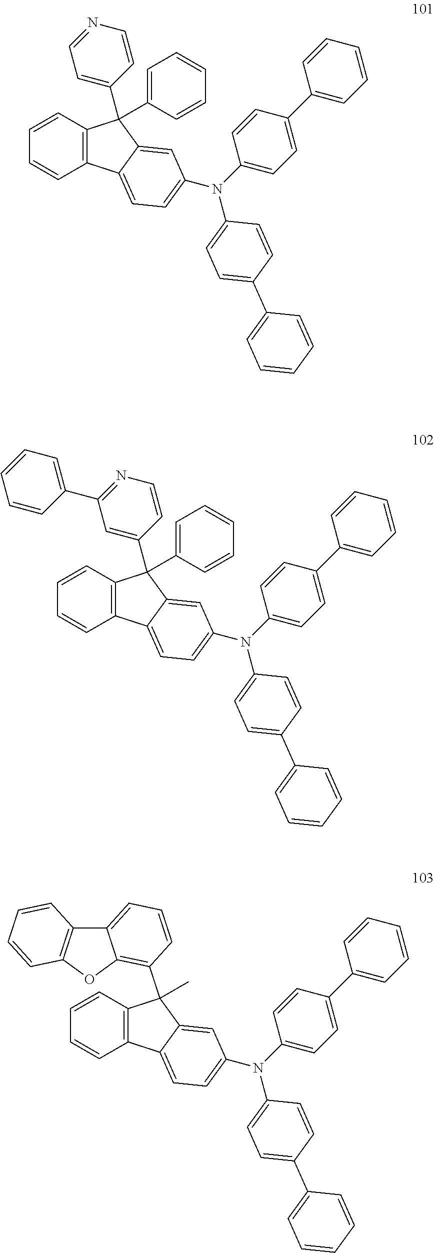 [SMILES: c1ccc(-c2ccc(N(c3ccc(-c4ccccc4)cc3)c3ccc4c(c3)C(c3ccccc3)(c3ccncc3)c3ccccc3-4)cc2)cc1 ; c1ccc(-c2ccc(N(c3ccc(-c4ccccc4)cc3)c3ccc4c(c3)C(c3ccccc3)(c3ccnc(-c5ccccc5)c3)c3ccccc3-4)cc2)cc1 ; CC1(c2cccc3c2oc2ccccc23)c2ccccc2-c2ccc(N(c3ccc(-c4ccccc4)cc3)c3ccc(-c4ccccc4)cc3)cc21]

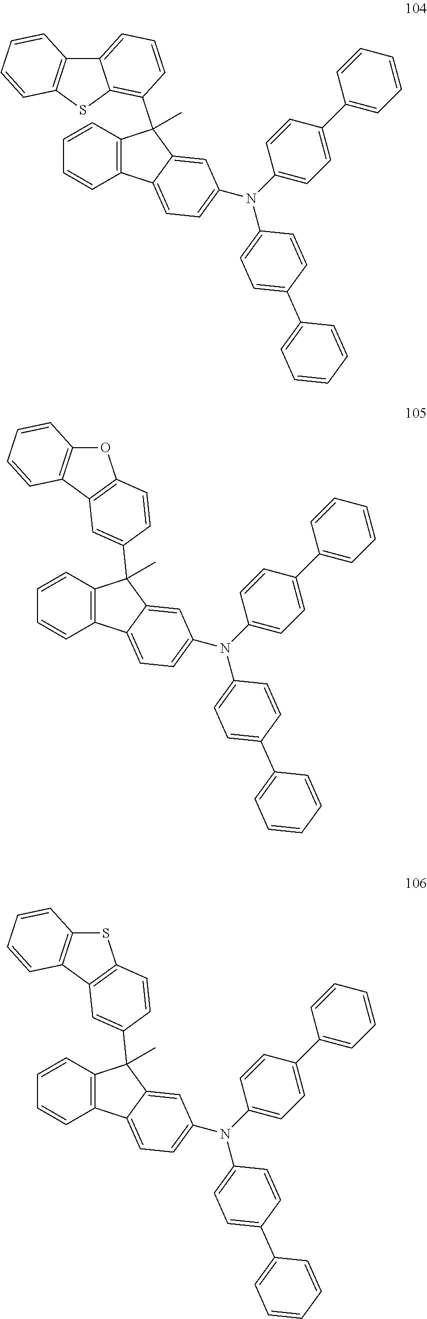 [SMILES: CC1(c2cccc3c2sc2ccccc23)c2ccccc2-c2ccc(N(c3ccc(-c4ccccc4)cc3)c3ccc(-c4ccccc4)cc3)cc21 ; CC1(c2ccc3oc4ccccc4c3c2)c2ccccc2-c2ccc(N(c3ccc(-c4ccccc4)cc3)c3ccc(-c4ccccc4)cc3)cc21 ; CC1(c2ccc3sc4ccccc4c3c2)c2ccccc2-c2ccc(N(c3ccc(-c4ccccc4)cc3)c3ccc(-c4ccccc4)cc3)cc21]

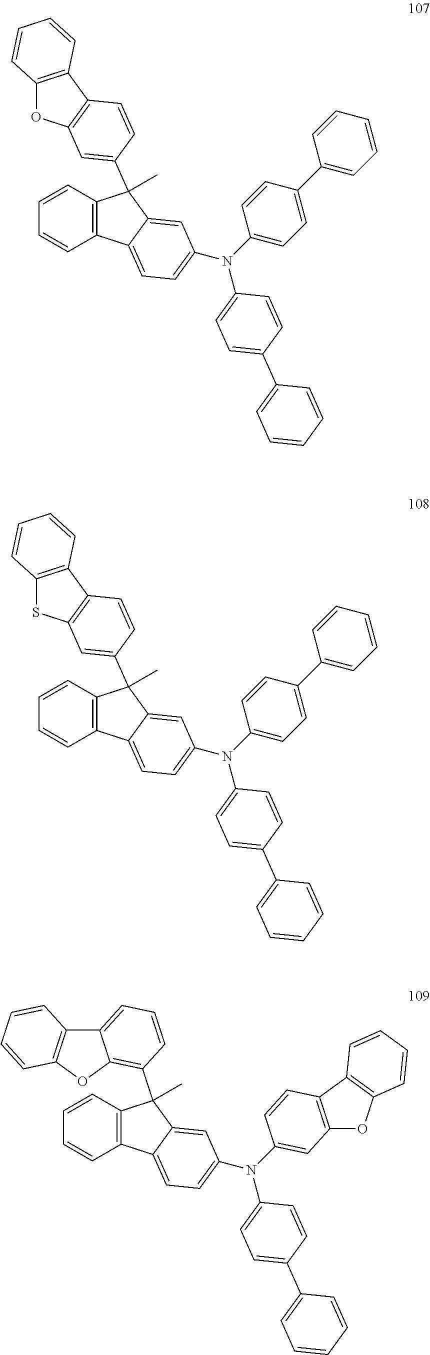 [SMILES: CC1(c2ccc3c(c2)oc2ccccc23)c2ccccc2-c2ccc(N(c3ccc(-c4ccccc4)cc3)c3ccc(-c4ccccc4)cc3)cc21 ; CC1(c2ccc3c(c2)sc2ccccc23)c2ccccc2-c2ccc(N(c3ccc(-c4ccccc4)cc3)c3ccc(-c4ccccc4)cc3)cc21 ; CC1(c2cccc3c2oc2ccccc23)c2ccccc2-c2ccc(N(c3ccc(-c4ccccc4)cc3)c3ccc4c(c3)oc3ccccc34)cc21]

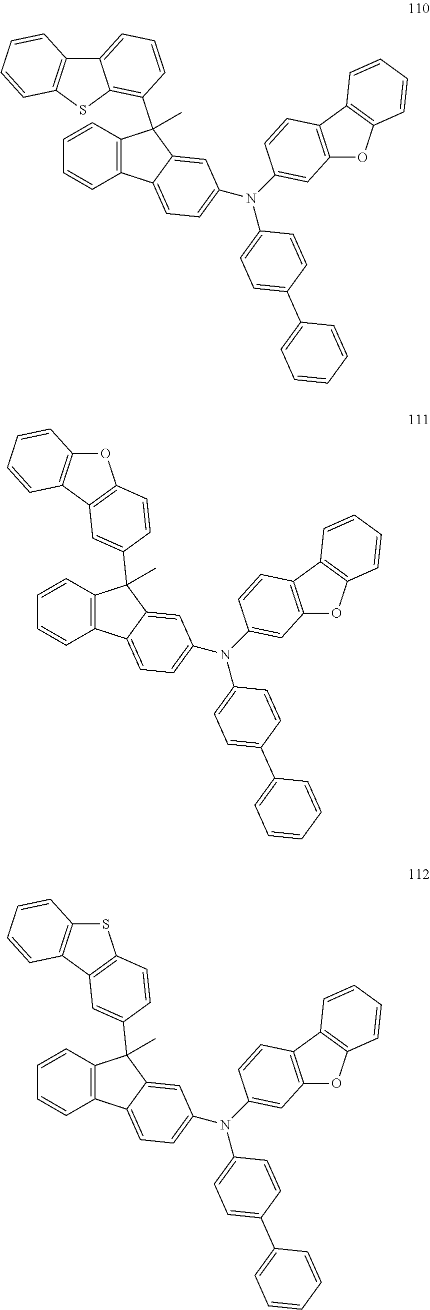 [SMILES: CC1(c2cccc3c2sc2ccccc23)c2ccccc2-c2ccc(N(c3ccc(-c4ccccc4)cc3)c3ccc4c(c3)oc3ccccc34)cc21 ; CC1(c2ccc3oc4ccccc4c3c2)c2ccccc2-c2ccc(N(c3ccc(-c4ccccc4)cc3)c3ccc4c(c3)oc3ccccc34)cc21 ; CC1(c2ccc3sc4ccccc4c3c2)c2ccccc2-c2ccc(N(c3ccc(-c4ccccc4)cc3)c3ccc4c(c3)oc3ccccc34)cc21]

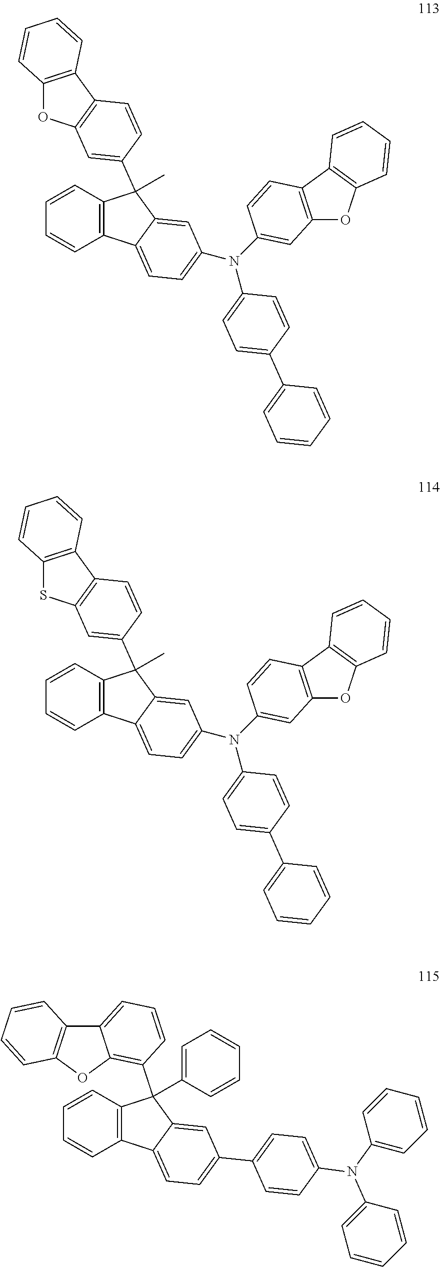 [SMILES: CC1(c2ccc3c(c2)oc2ccccc23)c2ccccc2-c2ccc(N(c3ccc(-c4ccccc4)cc3)c3ccc4c(c3)oc3ccccc34)cc21 ; CC1(c2ccc3c(c2)sc2ccccc23)c2ccccc2-c2ccc(N(c3ccc(-c4ccccc4)cc3)c3ccc4c(c3)oc3ccccc34)cc21 ; c1ccc(N(c2ccccc2)c2ccc(-c3ccc4c(c3)C(c3ccccc3)(c3cccc5c3oc3ccccc35)c3ccccc3-4)cc2)cc1]

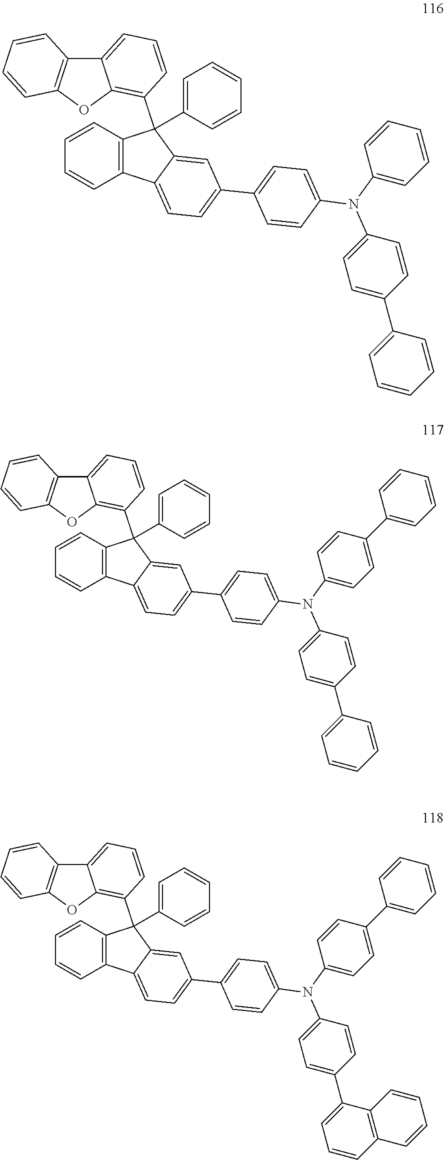 [SMILES: c1ccc(-c2ccc(N(c3ccccc3)c3ccc(-c4ccc5c(c4)C(c4ccccc4)(c4cccc6c4oc4ccccc46)c4ccccc4-5)cc3)cc2)cc1 ; c1ccc(-c2ccc(N(c3ccc(-c4ccccc4)cc3)c3ccc(-c4ccc5c(c4)C(c4ccccc4)(c4cccc6c4oc4ccccc46)c4ccccc4-5)cc3)cc2)cc1 ; c1ccc(-c2ccc(N(c3ccc(-c4ccc5c(c4)C(c4ccccc4)(c4cccc6c4oc4ccccc46)c4ccccc4-5)cc3)c3ccc(-c4cccc5ccccc45)cc3)cc2)cc1]

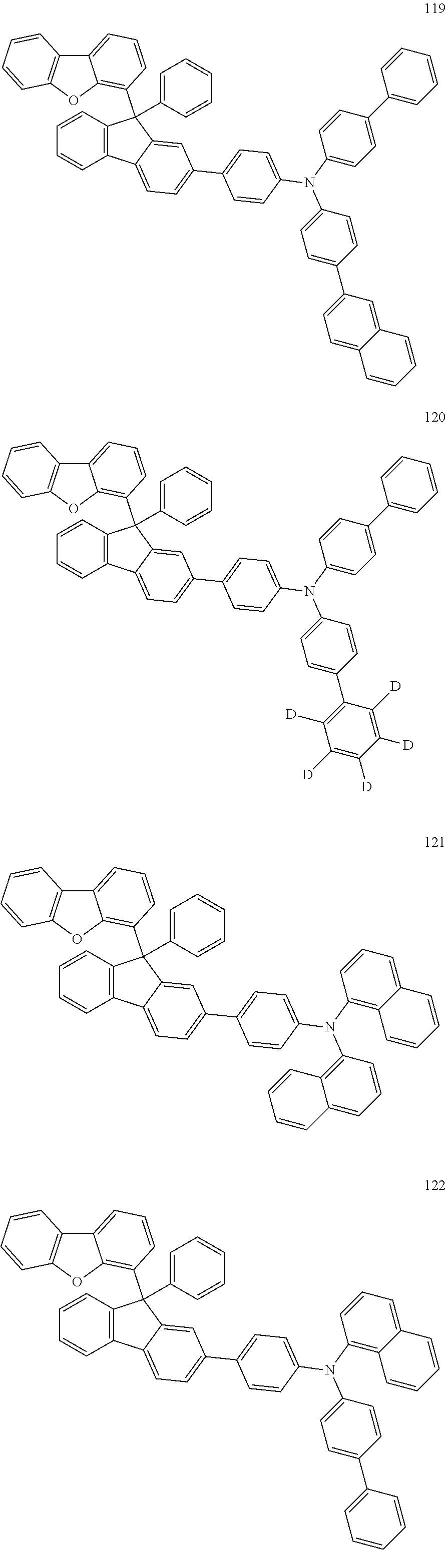 [SMILES: c1ccc(-c2ccc(N(c3ccc(-c4ccc5c(c4)C(c4ccccc4)(c4cccc6c4oc4ccccc46)c4ccccc4-5)cc3)c3ccc(-c4ccc5ccccc5c4)cc3)cc2)cc1 ; [2H]c1c([2H])c([2H])c(-c2ccc(N(c3ccc(-c4ccccc4)cc3)c3ccc(-c4ccc5c(c4)C(c4ccccc4)(c4cccc6c4oc4ccccc46)c4ccccc4-5)cc3)cc2)c([2H])c1[2H] ; c1ccc(C2(c3cccc4c3oc3ccccc34)c3ccccc3-c3ccc(-c4ccc(N(c5cccc6ccccc56)c5cccc6ccccc56)cc4)cc32)cc1 ; c1ccc(-c2ccc(N(c3ccc(-c4ccc5c(c4)C(c4ccccc4)(c4cccc6c4oc4ccccc46)c4ccccc4-5)cc3)c3cccc4ccccc34)cc2)cc1]

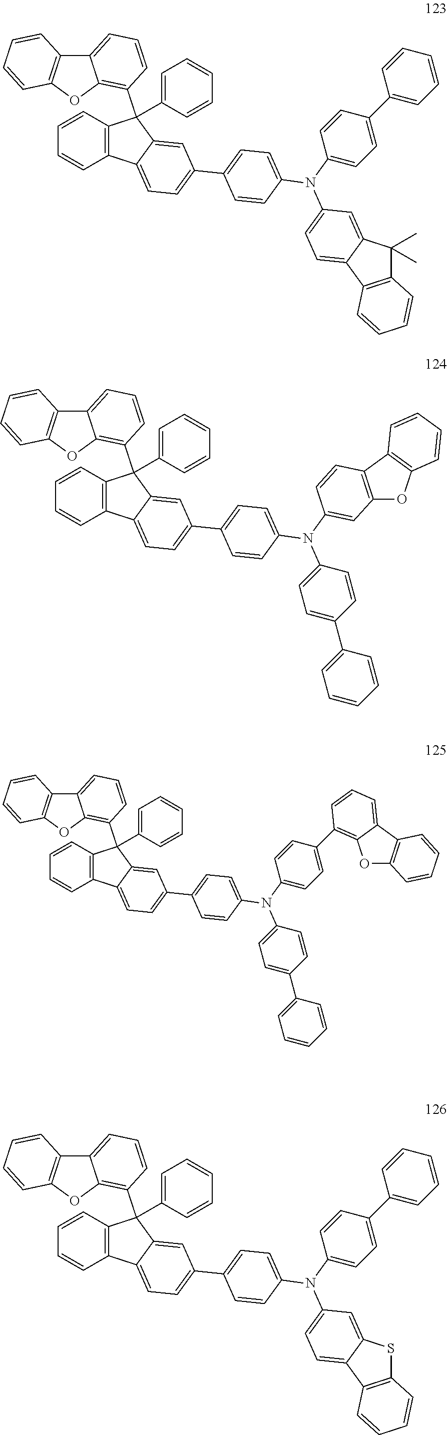 [SMILES: CC1(C)c2ccccc2-c2ccc(N(c3ccc(-c4ccccc4)cc3)c3ccc(-c4ccc5c(c4)C(c4ccccc4)(c4cccc6c4oc4ccccc46)c4ccccc4-5)cc3)cc21 ; c1ccc(-c2ccc(N(c3ccc(-c4ccc5c(c4)C(c4ccccc4)(c4cccc6c4oc4ccccc46)c4ccccc4-5)cc3)c3ccc4c(c3)oc3ccccc34)cc2)cc1 ; c1ccc(-c2ccc(N(c3ccc(-c4ccc5c(c4)C(c4ccccc4)(c4cccc6c4oc4ccccc46)c4ccccc4-5)cc3)c3ccc(-c4cccc5c4oc4ccccc45)cc3)cc2)cc1 ; c1ccc(-c2ccc(N(c3ccc(-c4ccc5c(c4)C(c4ccccc4)(c4cccc6c4oc4ccccc46)c4ccccc4-5)cc3)c3ccc4c(c3)sc3ccccc34)cc2)cc1]

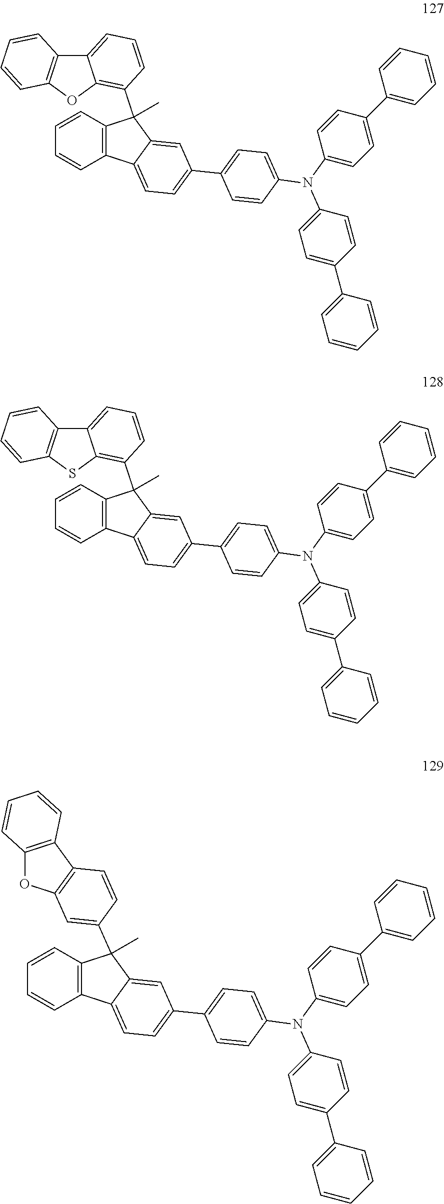 [SMILES: CC1(c2cccc3c2oc2ccccc23)c2ccccc2-c2ccc(-c3ccc(N(c4ccc(-c5ccccc5)cc4)c4ccc(-c5ccccc5)cc4)cc3)cc21 ; CC1(c2cccc3c2sc2ccccc23)c2ccccc2-c2ccc(-c3ccc(N(c4ccc(-c5ccccc5)cc4)c4ccc(-c5ccccc5)cc4)cc3)cc21 ; CC1(c2ccc3c(c2)oc2ccccc23)c2ccccc2-c2ccc(-c3ccc(N(c4ccc(-c5ccccc5)cc4)c4ccc(-c5ccccc5)cc4)cc3)cc21]

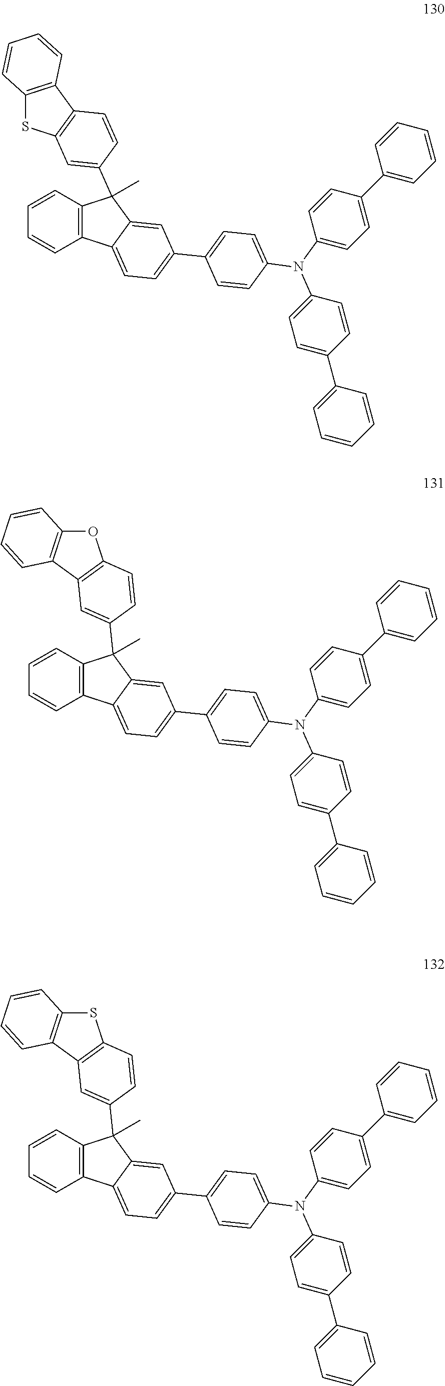 [SMILES: CC1(c2ccc3c(c2)sc2ccccc23)c2ccccc2-c2ccc(-c3ccc(N(c4ccc(-c5ccccc5)cc4)c4ccc(-c5ccccc5)cc4)cc3)cc21 ; CC1(c2ccc3oc4ccccc4c3c2)c2ccccc2-c2ccc(-c3ccc(N(c4ccc(-c5ccccc5)cc4)c4ccc(-c5ccccc5)cc4)cc3)cc21 ; CC1(c2ccc3sc4ccccc4c3c2)c2ccccc2-c2ccc(-c3ccc(N(c4ccc(-c5ccccc5)cc4)c4ccc(-c5ccccc5)cc4)cc3)cc21]

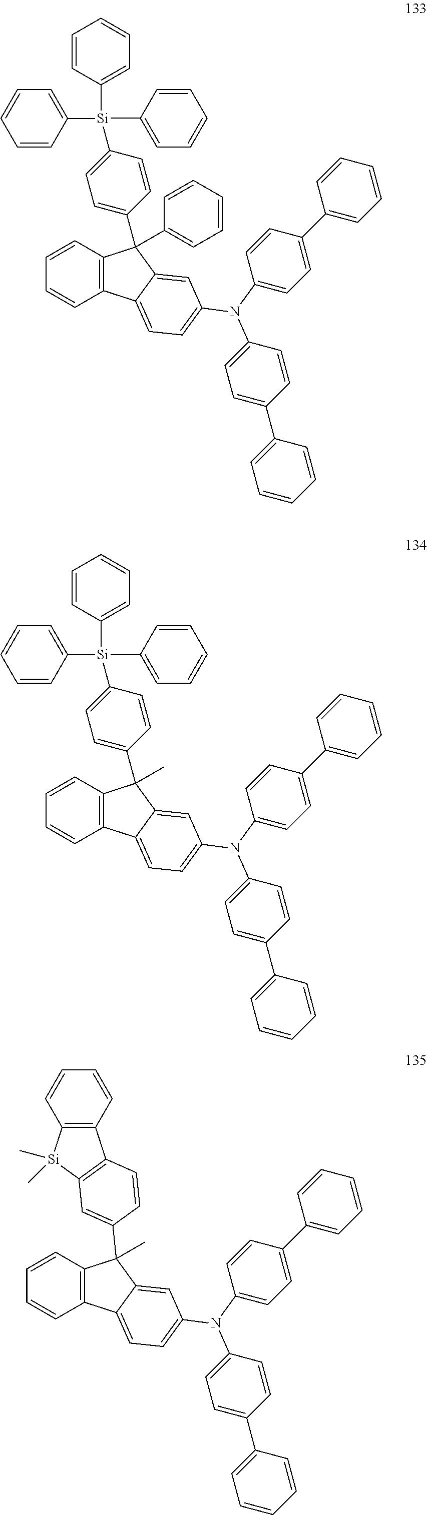 [SMILES: c1ccc(-c2ccc(N(c3ccc(-c4ccccc4)cc3)c3ccc4c(c3)C(c3ccccc3)(c3ccc([Si](c5ccccc5)(c5ccccc5)c5ccccc5)cc3)c3ccccc3-4)cc2)cc1 ; CC1(c2ccc([Si](c3ccccc3)(c3ccccc3)c3ccccc3)cc2)c2ccccc2-c2ccc(N(c3ccc(-c4ccccc4)cc3)c3ccc(-c4ccccc4)cc3)cc21 ; CC1(c2ccc3c(c2)[Si](C)(C)c2ccccc2-3)c2ccccc2-c2ccc(N(c3ccc(-c4ccccc4)cc3)c3ccc(-c4ccccc4)cc3)cc21]

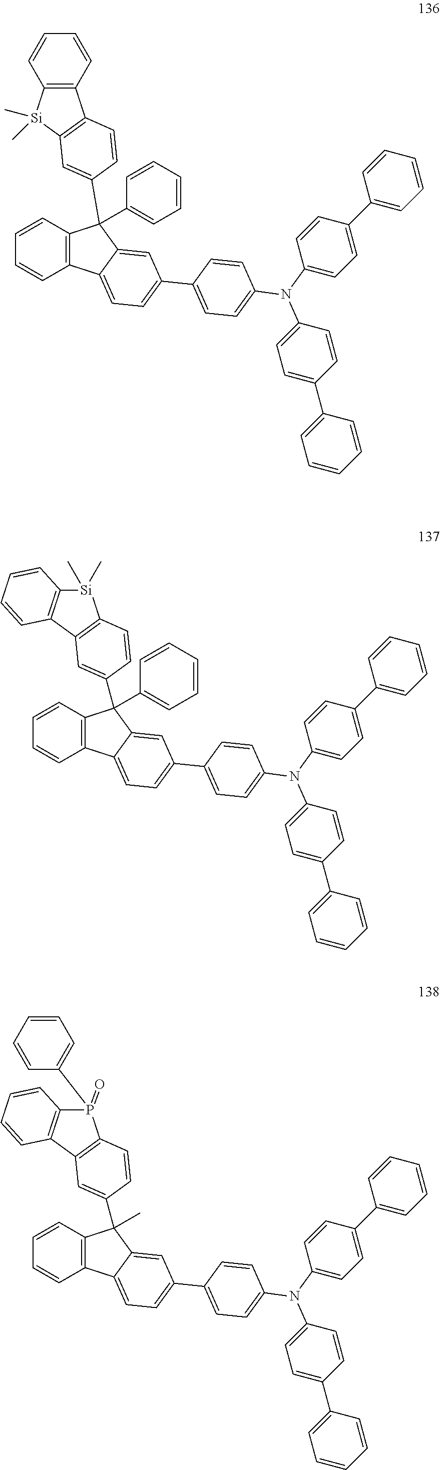 [SMILES: C[Si]1(C)c2ccccc2-c2ccc(C3(c4ccccc4)c4ccccc4-c4ccc(-c5ccc(N(c6ccc(-c7ccccc7)cc6)c6ccc(-c7ccccc7)cc6)cc5)cc43)cc21 ; C[Si]1(C)c2ccccc2-c2cc(C3(c4ccccc4)c4ccccc4-c4ccc(-c5ccc(N(c6ccc(-c7ccccc7)cc6)c6ccc(-c7ccccc7)cc6)cc5)cc43)ccc21 ; CC1(c2ccc3c(c2)-c2ccccc2P3(=O)c2ccccc2)c2ccccc2-c2ccc(-c3ccc(N(c4ccc(-c5ccccc5)cc4)c4ccc(-c5ccccc5)cc4)cc3)cc21]

The material for an organic EL device according to an embodiment of the present disclosure may be included in at least one layer selected from a plurality of organic layers forming the organic EL device. In some embodiments, the material may be included in at least one laminated layer between an emission layer and an anode in an organic EL device.

As described above, the material for an organic EL device according to an embodiment of the present disclosure may include a heteroaryl group HAr and a substituent Ar³ different from the heteroaryl group at position 9 of a fluorenyl group coupled or combined with an amine, such that the entire molecule is substantially asymmetric, and the material may be more amorphous. Accordingly, charge transportation may become smooth (e.g., may be improved), and the lifetime of the organic EL device may be improved.

### Organic EL Device

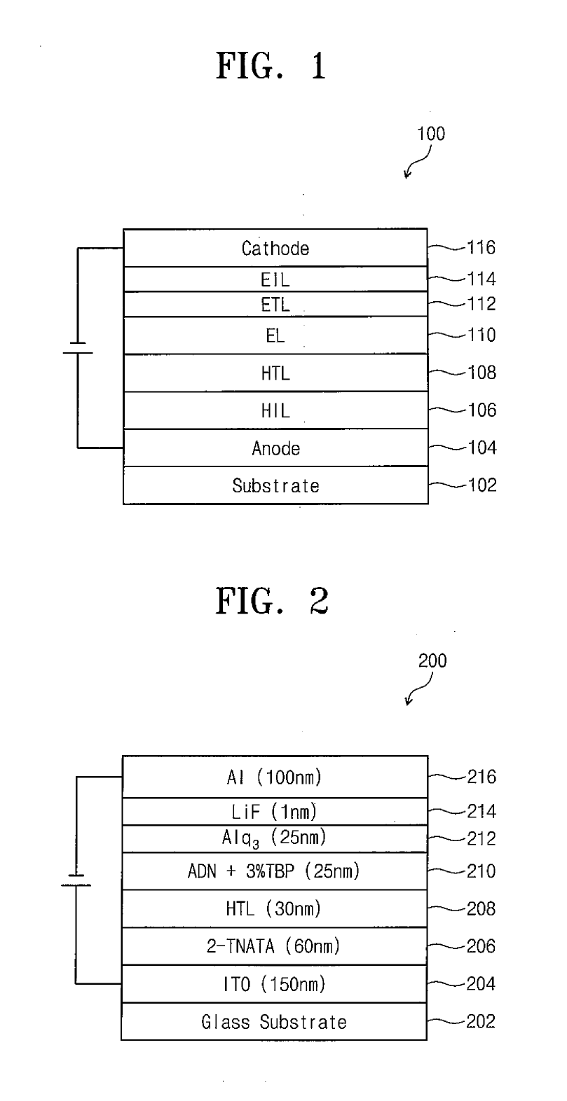

Hereinafter, an organic EL device using the material for an organic EL device according to an embodiment of the present disclosure will be explained. FIG. 1 is a schematic diagram illustrating an organic EL according to an embodiment of the present disclosure. The organic EL device 100 may include, for example, a substrate 102, an anode 104, a hole injection layer 106, a hole transport layer 108, an emission layer 110, an electron transport layer 112, an electron injection layer 114, and a cathode 116. In one or more embodiments, the material for an organic EL device may be used in at least one laminated layer between the emission layer and the anode.

In one embodiment, the material for an organic EL device according to the present disclosure is used in the hole transport layer 108.

The substrate 102 may be a transparent glass substrate, a semiconductor substrate formed using silicon, or a flexible substrate of a resin, etc.

The anode 104 may be on the substrate 102 and may be formed using indium tin oxide (ITO; In₂O₃—SnO₂), indium zinc oxide (IZO; In₂O₃—ZnO), etc.

The hole injection layer (HIL) 106 may be formed on the anode 104 to a thickness of about 10 nm to about 150 nm using any suitable material. The material may include, for example, triphenylamine-containing polyether ketone (TPAPEK), 4-isopropyl-4′-methyldiphenyliodonium tetrakis(pentafluorophenyl)borate (PPBI), N,N′-diphenyl-N,N′-bis-[4-(phenyl-m-tolyl-amino)-phenyl]-phenyl-4,4′-diamine (DNTPD), a phthalocyanine compound such as copper phthalocyanine, 4,4′,4″-tris(3-methylphenylphenylamino)triphenylamine (m-MTDATA), N,N′-di(1-naphthyl)-N,N′-diphenylbenzidine (NPB), 4,4′,4″-tris(N,N-diphenylamino)triphenylamine (TDATA), 4,4′,4″-tris(N,N-2-naphthylphenylamino)triphenylamine (2-TNATA), polyaniline/dodecylbenzenesulfonic acid (PANI/DBSA), poly(3,4-ethylenedioxythiophene)/poly(4-styrenesulfonate) (PEDOT/PSS), polyaniline/camphorsulfonic acid (PANI/CSA), polyaniline/poly(4-styrenesulfonate (PANI/PSS), etc.

The hole transport layer (HTL) 108 may be formed on the hole injection layer 106 to a thickness of about 3 nm to about 100 nm using the material for an organic EL device according to an embodiment of the present disclosure. The hole transport layer 108 including the material for an organic EL device according to an embodiment of the present disclosure may be formed by, for example, a vacuum evaporation method.

The emission layer (EL) 110 may be formed on the hole transport layer 108 to a thickness of about 10 nm to about 60 nm using any suitable host material. The host materials used in the emission layer 110 may include, for example, tris(8-quinolinolato)aluminum (Alq3), 4,4′-N,N′-dicarbazole-biphenyl (CBP), poly(N-vinylcarbazole) (PVK), anthracene derivatives such as 9,10-di(naphthalene-2-yl)anthracene (ADN), 4,4′,4″-tris(N-carbazolyl)triphenylamine (TCTA), 1,3,5-tris(N-phenylbenzimidazole-2-yl)benzene (TPBI), 3-tert-butyl-9,10-di(naphth-2-yl)anthracene (TBADN), distyrylarylene (DSA) and 4,4′-bis(9-carbazole)-2,2′-dimethyl-biphenyl (dmCBP).

The dopant material of the emission layer 110 may include styryl derivatives (such as 1,4-bis[2-(3-N-ethylcarbazolyl)vinyl]benzene (BCzVB), 4-(di-p-tolylamino)-4′-[(di-p-tolylamino)styryl]stilbene (DPAVB), and N-(4-((E)-2-(6-((E)-4-(diphenylamino)styryl)naphthalen-2-yl)vinyl)phenyl-N-phenylbenzeneamine (N-BDAVBI)), perylene and derivatives thereof (such as 2,5,8,11-tetra-t-butylperylene (TBPe)), pyrene and derivatives thereof (such as 1,1-dipyrene, 1,4-dipyrenylbenzene, and 1,4-bis(N,N-diphenylamino)pyrene), etc., without limitation.

The electron transport layer (ETL) 112 may be formed to a thickness of about 15 nm to about 50 nm on the emission layer 110 using, for example, tris(8-hydroxyquinolinolato)aluminum (Alq3) and/or a material having a nitrogen-containing aromatic ring (for example, a material including a pyridine ring such as 1,3,5-tri[(3-pyridyl)-phen-3-yl]benzene, a material including a triazine ring such as 2,4,6-tris[3′-(pyridine-3-yl)biphenyl-3-yl]1,3,5-triazine, and a material including an imidazole derivative such as 2-(4-N-phenylbenzoimidazolyl-1-ylphenyl)-9,10-dinaphthylanthracene).

The electron injection layer (EIL) 114 may be formed to a thickness of about 0.3 nm to about 9 nm on the electron transport layer 112 using, for example, a material including lithium fluoride (LiF), lithium-8-quinolinato (Liq), etc.

The cathode 116 may be on the electron injection layer 114 and may be formed using a metal such as aluminum (Al), silver (Ag), lithium (Li), magnesium (Mg) and calcium (Ca), and/or a transparent material such as ITO and IZO.

Each layer may be formed by selecting an appropriate or suitable layer forming method such as a vacuum evaporation method, a sputtering method, and/or other suitable coating methods, depending on the material to be used.

In the organic EL device 100, a hole transport layer capable of supporting long lifetimes may be formed using the material for an organic EL device according to an embodiment of the present disclosure.

In the organic EL device 100, the material for an organic EL device according to an embodiment of the present disclosure may be used as the material for a hole injection layer. As described above, an organic EL device with a long lifetime may be realized when the material for an organic EL device according to an embodiment of the present disclosure is included in at least one layer selected from a plurality of organic layers forming the organic EL device.

In some embodiments, the material for an organic EL device according to the present disclosure may be applied to an active matrix type (e.g., active matrix) organic EL display using thin film transistors (TFTs).

### Examples

### Preparation Method

A method of synthesizing the material for an organic EL device and a method of manufacturing the organic EL device according to the present disclosure will be explained in more detail. However, the following examples are only for illustration, and the scope of the present disclosure is not limited thereto.

### Synthetic Method of Compound 3

Compound 3 may be synthesized according to the following method. First, Compound A was synthesized as an intermediate.

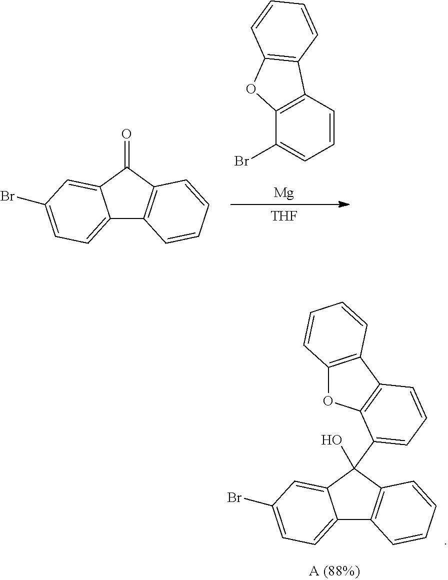

### Synthesis of Compound A

10 mL of a dehydrated (e.g., water-free) THF solution of 8.00 g (30.9 mmol) of magnesium metal was added to a 500 mL three-necked flask, followed by stirring at about 0° C. A dehydrated THF solution of 7.62 g (30.9 mmol) of 4-bromodibenzofuran was added dropwise, followed by stirring for about 2 hours at room temperature. 85 mL of a dehydrated THF solution of 8.00 g (30.9 mmol) of 3-bromobenzophenone was added dropwise, followed by stirring for about 2 hours and stirring for about 3 hours at room temperature. After reaction, a 1 N aqueous solution of NH₃Cl₄ was added to the reaction mixture, followed by stirring for about 1 hour. The reaction product was washed with water, and a resulting organic phase was concentrated to produce a candy-like substance. Finally, the substance was washed with methanol and dried to produce 11.6 g of Compound A as a white powder with yield of about 88%. The molecular weight of Compound A measured by Fast Atom Bombardment-Mass Spectrometry (FAB-MS) was 427.

Using Compound A as a raw material, Compound B was synthesized according to the following method.

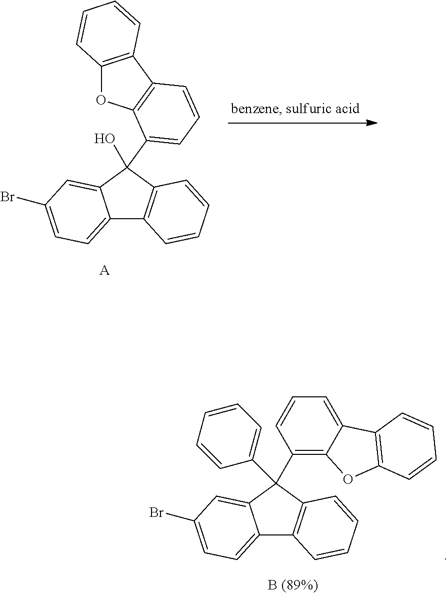

### Synthesis of Compound B

60 mL of a dehydrated benzene solution of 8.00 g (18.7 mmol) of Compound A was added to a 200 mL three-necked flask, followed by dropwise addition of 6.39 mL (39.4 mmol) of sulfuric acid and stirring at about 80° C. for about 2 hours. After reaction, a 1N aqueous solution of —NaHCO₃ was added to the reaction mixture, followed by stirring for about 1 hour. The product thus obtained was washed with water, and the resulting organic phase was concentrated to obtain a white solid. Finally, the white solid was washed with methanol and dried to produce 8.11 g of Compound B as a white powder with yield of about 89%. The molecular weight of Compound B measured by FAB-MS was 487.

Using Compound B as a raw material, Compound C as a final product was synthesized according to the following method.

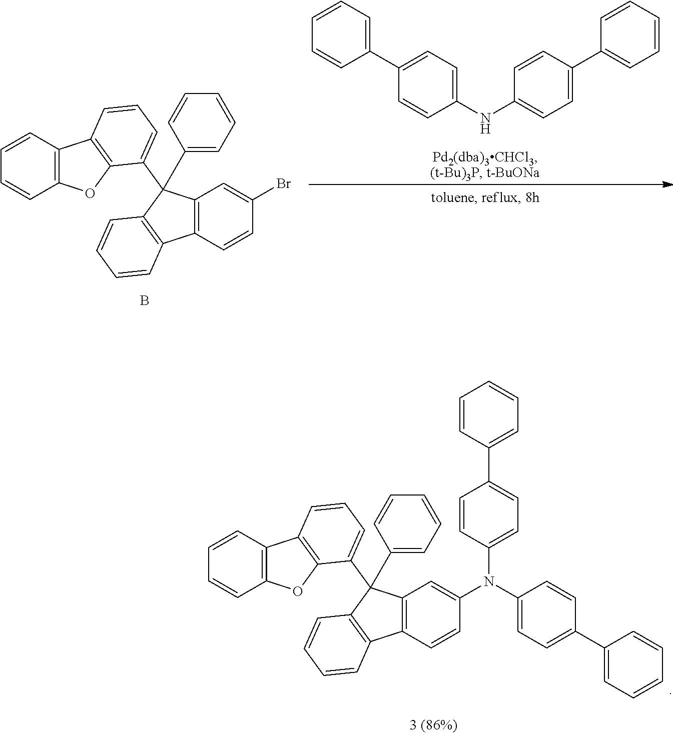

### Synthesis of Compound 3

Under an Argon atmosphere, 2.67 g of Compound B, 1.60 g of bis(4-biphenylyl)amine, 0.30 g of Pd₂(dba)₃, 0.20 g of tri-tert-butylphosphine and 1.44 g of NaOtBu were added to a 1,000 mL three-necked flask, followed by heating and refluxing in 40 mL of toluene for about 5 hours. After air cooling, water was added, the organic layer was separated, and the solvents were distilled. The crude product thus obtained was separated using silica gel column chromatography (using a mixture solvent of dichloromethane and hexane) and recrystallized using a mixed solvent of toluene/ethanol to produce 2.83 g of Compound 3 as a yellow solid (Yield 89%). The molecular weight of Compound 3 measured by FAB-MS was 728. Chemical shift values (δ) of Compound 3 measured by ¹H-NMR (CDCl₃) were 7.89-7.87 (m, 2H), 7.71 (d, 1H, J=7.60 Hz), 7.66 (d, 1H, J=7.60 Hz), 7.54-7.51 (m, 12H), 7.41-7.21 (m, 10H), 7.05 (d, 2H, J=7.80 Hz), 6.75 (s, 1H), 7.00-6.88 (m, 6H), 6.58 (d, 2H, J=7.80 Hz).

### Synthetic Method of Compound 15

Compound 15 was synthesized according to substantially the same method used to synthesize Compound 3, except for changing the 4-bromodibenzofuran used to synthesize Compound A to 4-bromodibenzothiophene. The molecular weight of Compound 15 measured by FAB-MS was 816. Chemical shift values (δ) of Compound 15 measured by ¹H-NMR (CDCl₃) were 7.99-7.95 (m, 2H), 7.71 (d, 1H, J=7.70 Hz), 7.69 (d, 1H, J=7.50 Hz), 7.56-7.48 (m, 12H), 7.38-7.21 (m, 7H), 7.00 (d, 2H, J=7.80 Hz), 6.85 (s, 1H), 6.75-6.66 (m, 4H), 6.57 (d, 2H, J=7.80 Hz).

### Synthetic Method of Compound 27

Compound 27 was synthesized according to substantially the same method used to synthesize Compound 3, except for changing the 4-bromodibenzofuran used to synthesize Compound A to 2-bromodibenzofuran. The molecular weight of Compound 27 measured by FAB-MS was 584. Chemical shift values (δ) of Compound 27 measured by ¹H-NMR (CDCl₃) were 7.89-7.87 (m, 2H), 7.71 (d, 1H, J=7.60 Hz), 7.66 (d, 1H, J=7.60 Hz), 7.54-7.51 (m, 12H), 7.41-7.21 (m, 7H), 7.05 (d, 2H, J=7.80 Hz), 6.75 (s, 1H), 6.70-6.66 (m, 4H), 6.58 (d, 2H, J=7.80 Hz), 2.22 (s, 3H).

Organic EL devices according to Examples 1 to 3 were manufactured using the resulting Compounds 3, 15 and 27 as hole transport materials.

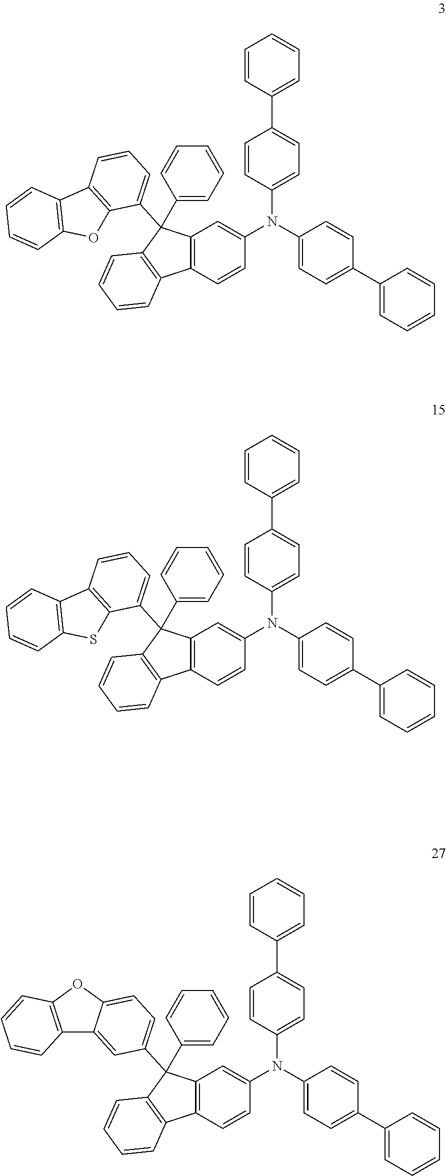 [SMILES: c1ccc(-c2ccc(N(c3ccc(-c4ccccc4)cc3)c3ccc4c(c3)C(c3ccccc3)(c3cccc5c3oc3ccccc35)c3ccccc3-4)cc2)cc1 ; c1ccc(-c2ccc(N(c3ccc(-c4ccccc4)cc3)c3ccc4c(c3)C(c3ccccc3)(c3cccc5c3sc3ccccc35)c3ccccc3-4)cc2)cc1 ; c1ccc(-c2ccc(N(c3ccc(-c4ccccc4)cc3)c3ccc4c(c3)C(c3ccccc3)(c3ccc5oc6ccccc6c5c3)c3ccccc3-4)cc2)cc1]

For comparison, organic EL devices according to Comparative Examples 1 to 3 were manufactured using Comparative Compounds C-1 to C-3 as hole transport materials.

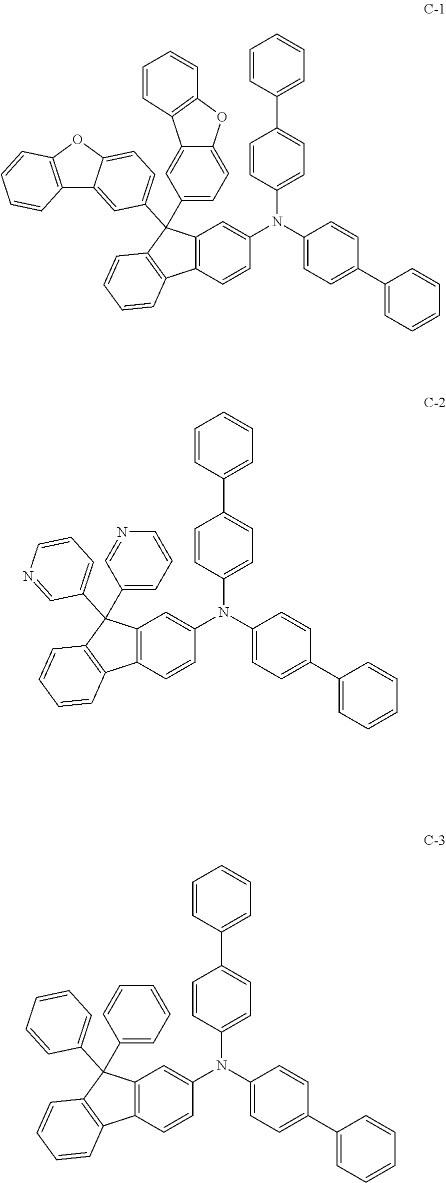 [SMILES: c1ccc(-c2ccc(N(c3ccc(-c4ccccc4)cc3)c3ccc4c(c3)C(c3ccc5oc6ccccc6c5c3)(c3ccc5oc6ccccc6c5c3)c3ccccc3-4)cc2)cc1 ; c1ccc(-c2ccc(N(c3ccc(-c4ccccc4)cc3)c3ccc4c(c3)C(c3cccnc3)(c3cccnc3)c3ccccc3-4)cc2)cc1 ; c1ccc(-c2ccc(N(c3ccc(-c4ccccc4)cc3)c3ccc4c(c3)C(c3ccccc3)(c3ccccc3)c3ccccc3-4)cc2)cc1]

An organic EL device 200 according to an embodiment of the present disclosure is shown in FIG. 2. In this embodiment, a substrate 202 was formed using a transparent glass substrate, an anode 204 was formed using ITO to a thickness of about 150 nm, a hole injection layer 206 was formed using 2-TNATA to a thickness of about 60 nm, a hole transport layer 208 was formed to a thickness of about 30 nm, an emission layer 210 was formed using ADN doped with 3% TBP to a thickness of about 25 nm, a hole transport layer 212 was formed using Alq3 to a thickness of about 25 nm, an electron injection layer 214 was formed using LiF to a thickness of about 1 nm, and a cathode 216 was formed using Al to a thickness of about 100 nm.

The half-lives of the organic EL devices 200 thus manufactured were evaluated. The half-life of each device was measured on the basis of an initial luminance of about 1,000 cd/m². Evaluation results are shown in Table 1. In Table 1, the luminance half-life of each Example and each Comparative Example is given as a relative value normalized to that of Comparative Example 1.

TABLE 1

| Device | | |
|---|---|---|
| Manufacturing Example | Hole Transport Material | Half Life |
| Example 1 | Compound 3 | 1.7 |
| Example 2 | Compound 15 | 1.5 |
| Example 3 | Compound 27 | 1.7 |
| Comparative Example 1 | Comparative Compound C-1 | 1.0 |
| Comparative Example 2 | Comparative Compound C-2 | 0.7 |
| Comparative Example 3 | Comparative Compound C-3 | 0.7 |

Referring to the results in Table 1, the organic EL devices of Examples 1 to 3 had improved (e.g., increased) lifetimes (long half-lives) when compared to the organic EL devices of Comparative Examples 1 to 3. In Examples 1 to 3, the material for an organic EL device according to an embodiment of the present disclosure includes a heteroaryl group HAr and a substituent Ar³ different from the heteroaryl group at position 9 of a fluorenyl group combined or coupled with an amine moiety, such that the entire molecule is substantially asymmetric, and the material may be more amorphous. Accordingly, charge transportation may become smooth (e.g., may be improved), and the lifetime of an organic EL device may be improved. In Comparative Examples 1 to 3, steric repulsion may be generated by the unshared electron pairs (e.g., lone electron pairs) on the dibenzofuran ring and/or nitrogen, and the lifetimes of the organic EL devices are shortened (e.g., decreased).

From the results in Table 1, it is recognized that organic EL devices using the material for an organic EL device, according to an embodiment of the present disclosure, have longer lifetimes when compared to the organic EL devices using the Comparative Compounds according to the Comparative Examples. The material for an organic EL device according to an embodiment of the present disclosure includes a heteroaryl group HAr and a substituent Ar³ different from the heteroaryl group at position 9 of a fluorenyl group combined or coupled with an amine moiety, and the lifetime of the organic EL device including the material may be increased or improved.

The material for an organic EL device according to the present disclosure has a wide energy gap, and application of the material in OLEDs in the red and/or green emission regions may be possible.

According to one or more embodiments of the present disclosure, a material for an organic EL device with a long lifetime and an organic EL device using the same are provided. The material for an organic EL device includes a heteroaryl group and a substituent different from the heteroaryl group at position 9 of a fluorenyl group combined or coupled with an amine. Accordingly, charge transportation may become smooth (e.g., may be improved), and the lifetime of the organic EL device may be improved. The above-described effects may be remarkable in the blue emission region.

While one or more example embodiments have been described with reference to the drawings, it will be understood by those of ordinary skill in the art that various changes in form and details may be made therein without departing from the spirit and scope of the present disclosure as defined by the following claims and equivalents thereof.

As used herein, expressions such as “at least one of”, “one of”, “at least one selected from”, and “one selected from”, when preceding a list of elements, modify the entire list of elements and do not modify the individual elements of the list. Further, the use of “may” when describing embodiments of the present disclosure refers to “one or more embodiments of the present disclosure”.

In addition, as used herein, the terms “use”, “using”, and “used” may be considered synonymous with the terms “utilize”, “utilizing”, and “utilized”, respectively.

As used herein, the terms “substantially”, “about”, and similar terms are used as terms of approximation and not as terms of degree, and are intended to account for the inherent deviations in measured or calculated values that would be recognized by those of ordinary skill in the art.

Also, any numerical range recited herein is intended to include all subranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

The above-disclosed subject matter is to be considered illustrative and not restrictive, and the appended claims and equivalents thereof are intended to cover all such modifications, enhancements, and other embodiments, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

What is claimed is:

1. A material for an organic electroluminescent (EL) device represented by Formula 1: 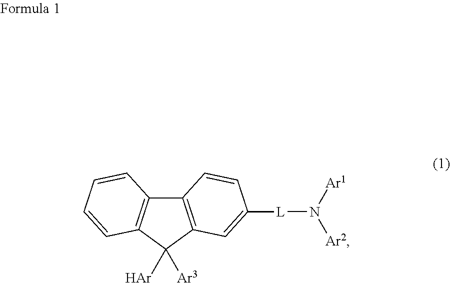
wherein in Formula 1,
Ar¹ and Ar² are each independently selected from a substituted or unsubstituted aryl group having 6 to 30 carbon atoms for forming a ring, and a substituted or unsubstituted heteroaryl group having 5 to 30 carbon atoms for forming a ring,
Ar³ is selected from a substituted or unsubstituted aryl group having 6 to 30 carbon atoms for forming a ring, a substituted or unsubstituted heteroaryl group having 5 to 30 carbon atoms for forming a ring, and an alkyl group having 1 to 6 carbon atoms,
HAr is a substituted or unsubstituted heteroaryl group having 5 to 30 carbon atoms for forming a ring,
L is selected from a direct linkage, a substituted or unsubstituted arylene group having 6 to 30 carbon atoms for forming a ring, and a substituted or unsubstituted heteroarylene group having 5 to 30 carbon atoms, and
HAr and Ar³ are different from each other.

2. The material of claim 1, wherein Ar¹ to Ar³ are each independently a substituted or unsubstituted aryl group having 6 to 30 carbon atoms for forming a ring.

3. The material of claim 1, wherein HAr is selected from a dibenzofuryl group and a dibenzothienyl group.

4. The material of claim 1, wherein the material for an organic EL device represented by Formula 1 is at least one selected from the following Compounds 1 to 132 and 135 to 138:

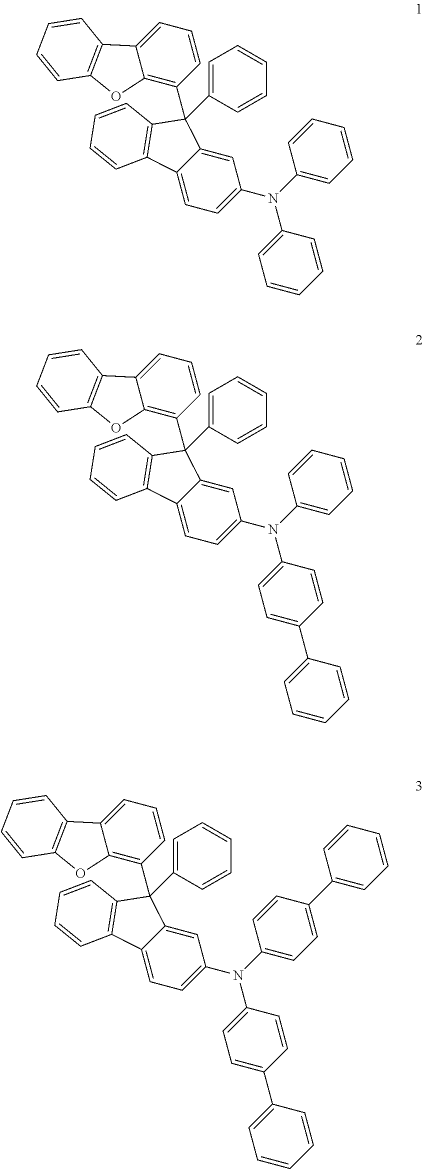 [SMILES: c1ccc(N(c2ccccc2)c2ccc3c(c2)C(c2ccccc2)(c2cccc4c2oc2ccccc24)c2ccccc2-3)cc1 ; c1ccc(-c2ccc(N(c3ccccc3)c3ccc4c(c3)C(c3ccccc3)(c3cccc5c3oc3ccccc35)c3ccccc3-4)cc2)cc1 ; c1ccc(-c2ccc(N(c3ccc(-c4ccccc4)cc3)c3ccc4c(c3)C(c3ccccc3)(c3cccc5c3oc3ccccc35)c3ccccc3-4)cc2)cc1]

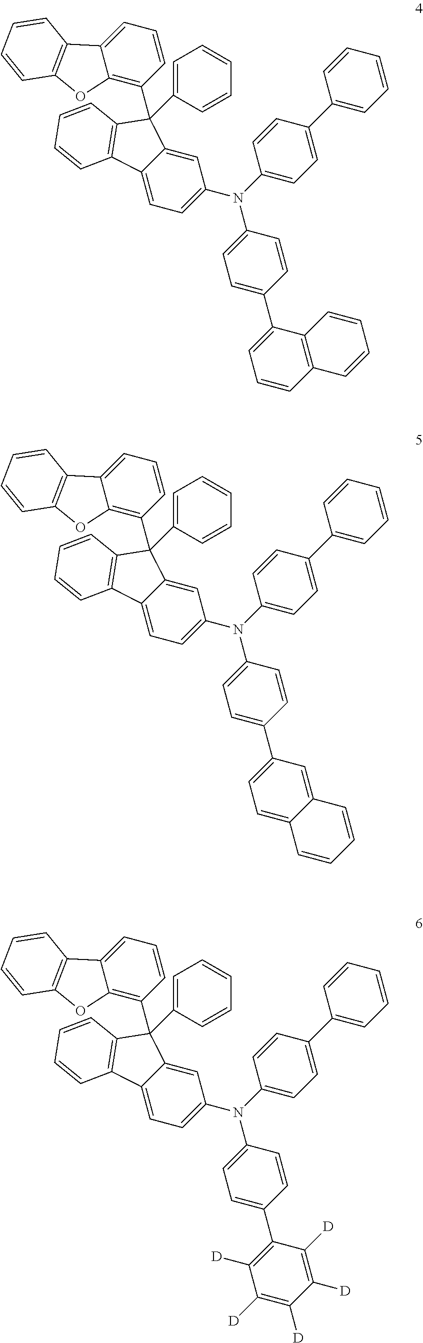 [SMILES: c1ccc(-c2ccc(N(c3ccc(-c4cccc5ccccc45)cc3)c3ccc4c(c3)C(c3ccccc3)(c3cccc5c3oc3ccccc35)c3ccccc3-4)cc2)cc1 ; c1ccc(-c2ccc(N(c3ccc(-c4ccc5ccccc5c4)cc3)c3ccc4c(c3)C(c3ccccc3)(c3cccc5c3oc3ccccc35)c3ccccc3-4)cc2)cc1 ; [2H]c1c([2H])c([2H])c(-c2ccc(N(c3ccc(-c4ccccc4)cc3)c3ccc4c(c3)C(c3ccccc3)(c3cccc5c3oc3ccccc35)c3ccccc3-4)cc2)c([2H])c1[2H]]

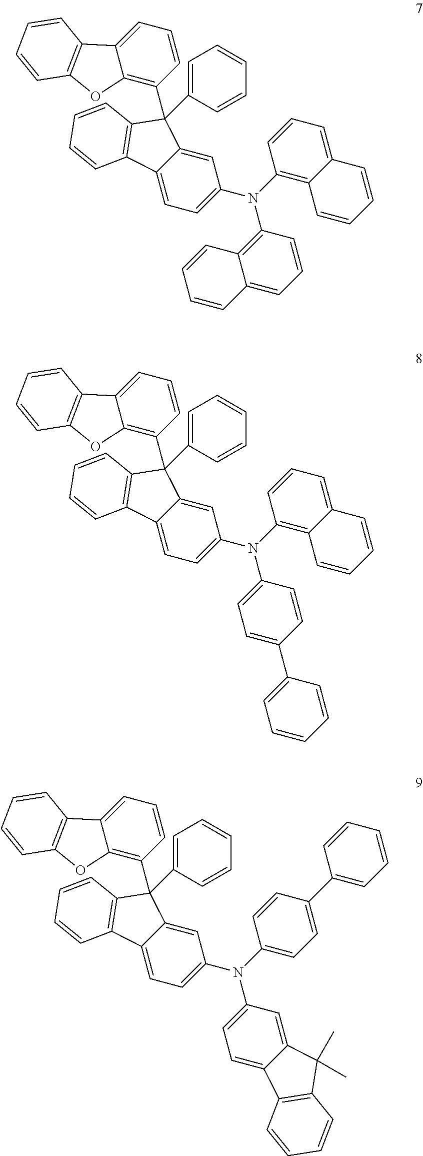 [SMILES: c1ccc(C2(c3cccc4c3oc3ccccc34)c3ccccc3-c3ccc(N(c4cccc5ccccc45)c4cccc5ccccc45)cc32)cc1 ; c1ccc(-c2ccc(N(c3ccc4c(c3)C(c3ccccc3)(c3cccc5c3oc3ccccc35)c3ccccc3-4)c3cccc4ccccc34)cc2)cc1 ; CC1(C)c2ccccc2-c2ccc(N(c3ccc(-c4ccccc4)cc3)c3ccc4c(c3)C(c3ccccc3)(c3cccc5c3oc3ccccc35)c3ccccc3-4)cc21]

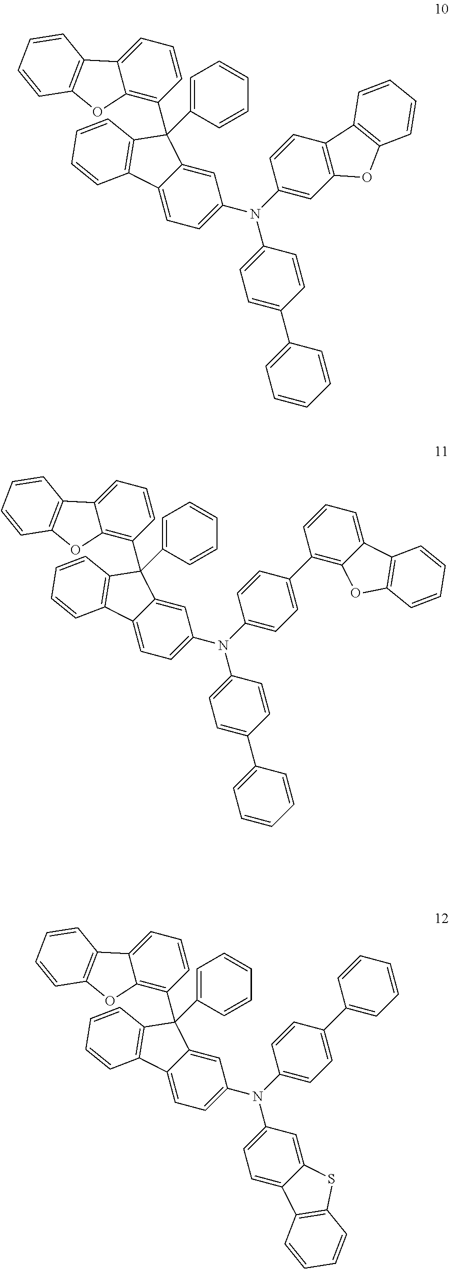 [SMILES: c1ccc(-c2ccc(N(c3ccc4c(c3)C(c3ccccc3)(c3cccc5c3oc3ccccc35)c3ccccc3-4)c3ccc4c(c3)oc3ccccc34)cc2)cc1 ; c1ccc(-c2ccc(N(c3ccc(-c4cccc5c4oc4ccccc45)cc3)c3ccc4c(c3)C(c3ccccc3)(c3cccc5c3oc3ccccc35)c3ccccc3-4)cc2)cc1 ; c1ccc(-c2ccc(N(c3ccc4c(c3)C(c3ccccc3)(c3cccc5c3oc3ccccc35)c3ccccc3-4)c3ccc4c(c3)sc3ccccc34)cc2)cc1]

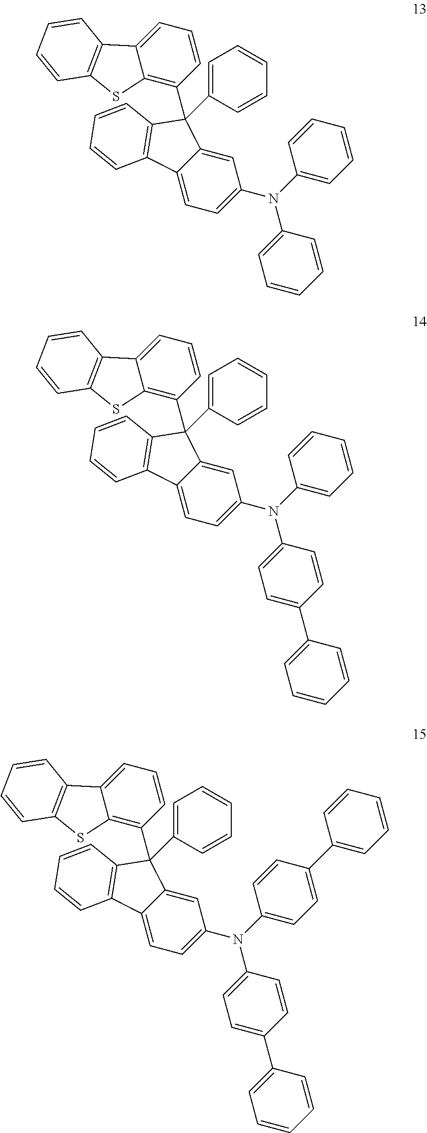 [SMILES: c1ccc(N(c2ccccc2)c2ccc3c(c2)C(c2ccccc2)(c2cccc4c2sc2ccccc24)c2ccccc2-3)cc1 ; c1ccc(-c2ccc(N(c3ccccc3)c3ccc4c(c3)C(c3ccccc3)(c3cccc5c3sc3ccccc35)c3ccccc3-4)cc2)cc1 ; c1ccc(-c2ccc(N(c3ccc(-c4ccccc4)cc3)c3ccc4c(c3)C(c3ccccc3)(c3cccc5c3sc3ccccc35)c3ccccc3-4)cc2)cc1]

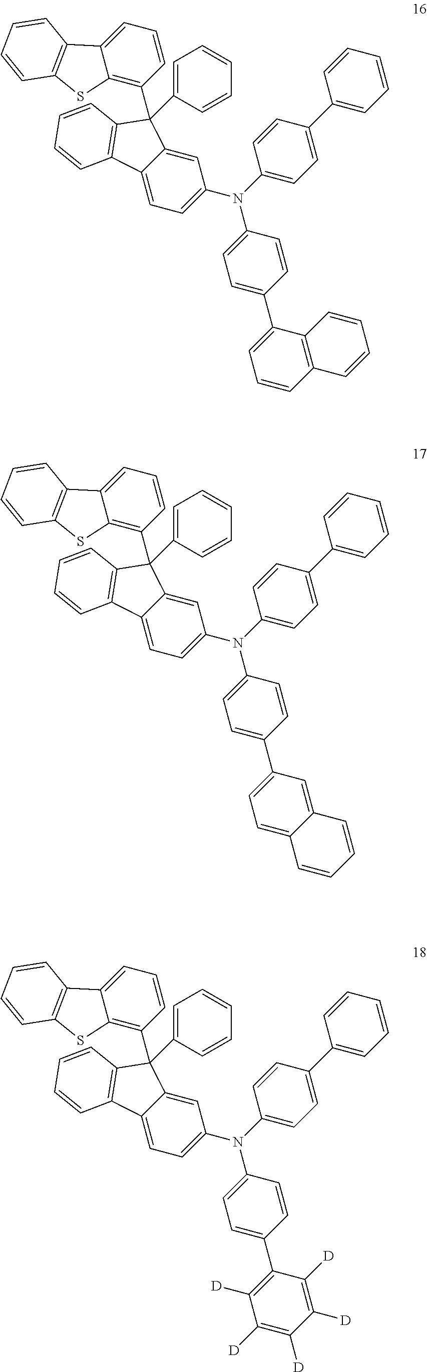 [SMILES: c1ccc(-c2ccc(N(c3ccc(-c4cccc5ccccc45)cc3)c3ccc4c(c3)C(c3ccccc3)(c3cccc5c3sc3ccccc35)c3ccccc3-4)cc2)cc1 ; c1ccc(-c2ccc(N(c3ccc(-c4ccc5ccccc5c4)cc3)c3ccc4c(c3)C(c3ccccc3)(c3cccc5c3sc3ccccc35)c3ccccc3-4)cc2)cc1 ; [2H]c1c([2H])c([2H])c(-c2ccc(N(c3ccc(-c4ccccc4)cc3)c3ccc4c(c3)C(c3ccccc3)(c3cccc5c3sc3ccccc35)c3ccccc3-4)cc2)c([2H])c1[2H]]

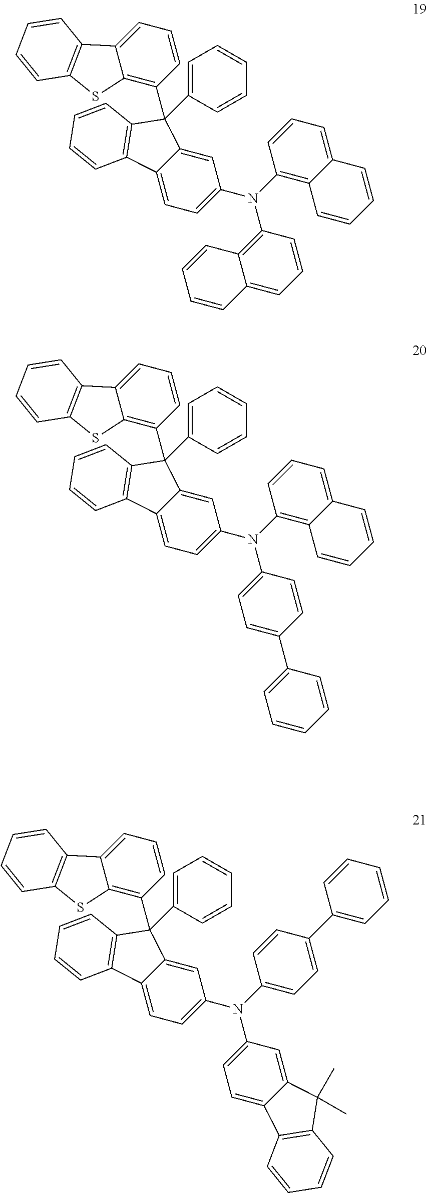 [SMILES: c1ccc(C2(c3cccc4c3sc3ccccc34)c3ccccc3-c3ccc(N(c4cccc5ccccc45)c4cccc5ccccc45)cc32)cc1 ; c1ccc(-c2ccc(N(c3ccc4c(c3)C(c3ccccc3)(c3cccc5c3sc3ccccc35)c3ccccc3-4)c3cccc4ccccc34)cc2)cc1 ; CC1(C)c2ccccc2-c2ccc(N(c3ccc(-c4ccccc4)cc3)c3ccc4c(c3)C(c3ccccc3)(c3cccc5c3sc3ccccc35)c3ccccc3-4)cc21]

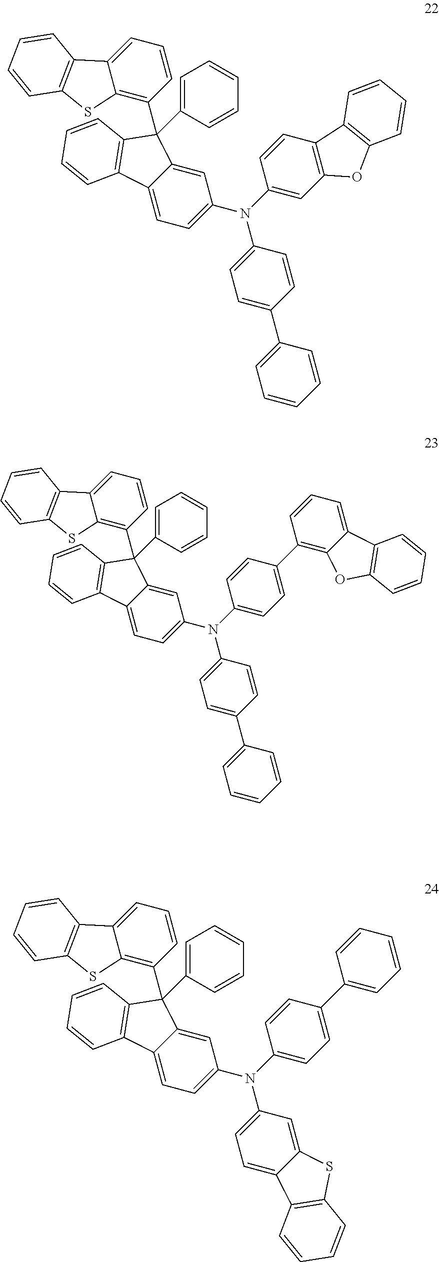 [SMILES: c1ccc(-c2ccc(N(c3ccc4c(c3)C(c3ccccc3)(c3cccc5c3sc3ccccc35)c3ccccc3-4)c3ccc4c(c3)oc3ccccc34)cc2)cc1 ; c1ccc(-c2ccc(N(c3ccc(-c4cccc5c4oc4ccccc45)cc3)c3ccc4c(c3)C(c3ccccc3)(c3cccc5c3sc3ccccc35)c3ccccc3-4)cc2)cc1 ; c1ccc(-c2ccc(N(c3ccc4c(c3)C(c3ccccc3)(c3cccc5c3sc3ccccc35)c3ccccc3-4)c3ccc4c(c3)sc3ccccc34)cc2)cc1]

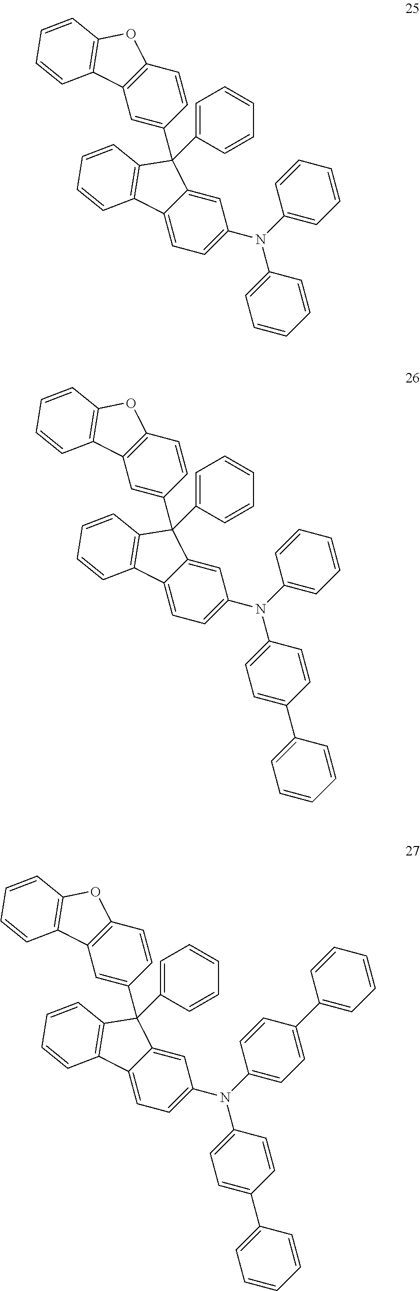 [SMILES: c1ccc(N(c2ccccc2)c2ccc3c(c2)C(c2ccccc2)(c2ccc4oc5ccccc5c4c2)c2ccccc2-3)cc1 ; c1ccc(-c2ccc(N(c3ccccc3)c3ccc4c(c3)C(c3ccccc3)(c3ccc5oc6ccccc6c5c3)c3ccccc3-4)cc2)cc1 ; c1ccc(-c2ccc(N(c3ccc(-c4ccccc4)cc3)c3ccc4c(c3)C(c3ccccc3)(c3ccc5oc6ccccc6c5c3)c3ccccc3-4)cc2)cc1]

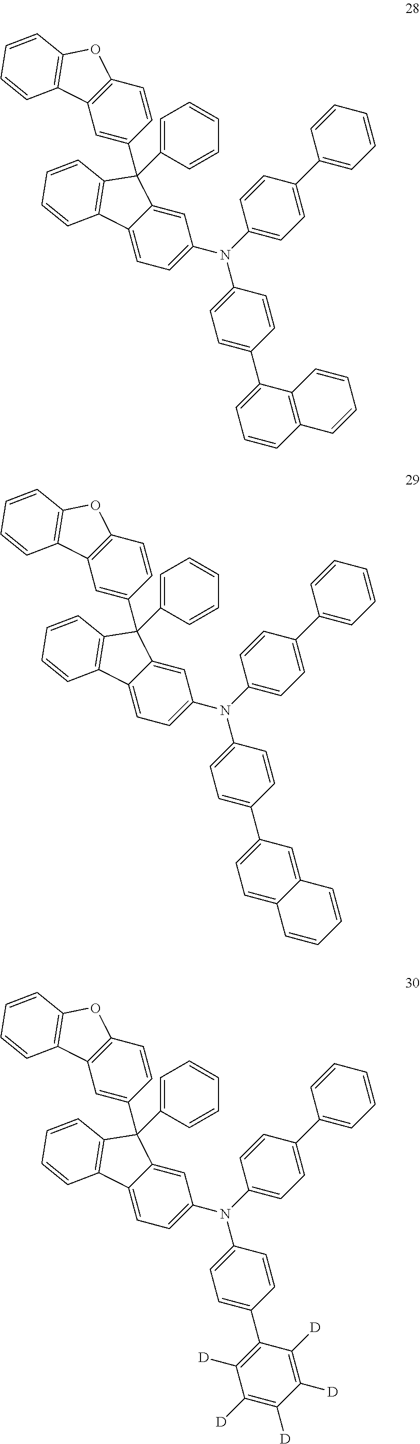 [SMILES: c1ccc(-c2ccc(N(c3ccc(-c4cccc5ccccc45)cc3)c3ccc4c(c3)C(c3ccccc3)(c3ccc5oc6ccccc6c5c3)c3ccccc3-4)cc2)cc1 ; c1ccc(-c2ccc(N(c3ccc(-c4ccc5ccccc5c4)cc3)c3ccc4c(c3)C(c3ccccc3)(c3ccc5oc6ccccc6c5c3)c3ccccc3-4)cc2)cc1 ; [2H]c1c([2H])c([2H])c(-c2ccc(N(c3ccc(-c4ccccc4)cc3)c3ccc4c(c3)C(c3ccccc3)(c3ccc5oc6ccccc6c5c3)c3ccccc3-4)cc2)c([2H])c1[2H]]

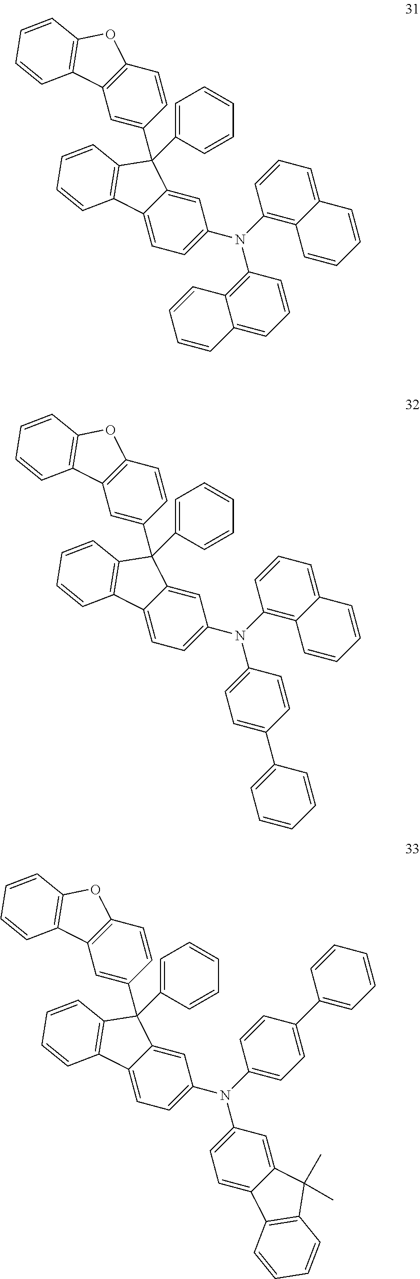 [SMILES: c1ccc(C2(c3ccc4oc5ccccc5c4c3)c3ccccc3-c3ccc(N(c4cccc5ccccc45)c4cccc5ccccc45)cc32)cc1 ; c1ccc(-c2ccc(N(c3ccc4c(c3)C(c3ccccc3)(c3ccc5oc6ccccc6c5c3)c3ccccc3-4)c3cccc4ccccc34)cc2)cc1 ; CC1(C)c2ccccc2-c2ccc(N(c3ccc(-c4ccccc4)cc3)c3ccc4c(c3)C(c3ccccc3)(c3ccc5oc6ccccc6c5c3)c3ccccc3-4)cc21]

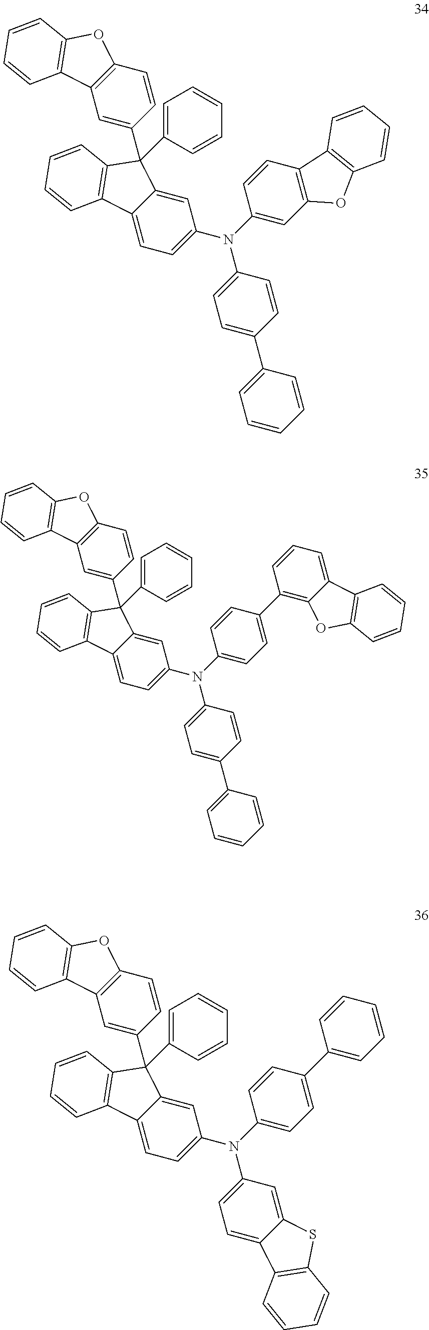 [SMILES: c1ccc(-c2ccc(N(c3ccc4c(c3)C(c3ccccc3)(c3ccc5oc6ccccc6c5c3)c3ccccc3-4)c3ccc4c(c3)oc3ccccc34)cc2)cc1 ; c1ccc(-c2ccc(N(c3ccc(-c4cccc5c4oc4ccccc45)cc3)c3ccc4c(c3)C(c3ccccc3)(c3ccc5oc6ccccc6c5c3)c3ccccc3-4)cc2)cc1 ; c1ccc(-c2ccc(N(c3ccc4c(c3)C(c3ccccc3)(c3ccc5oc6ccccc6c5c3)c3ccccc3-4)c3ccc4c(c3)sc3ccccc34)cc2)cc1]

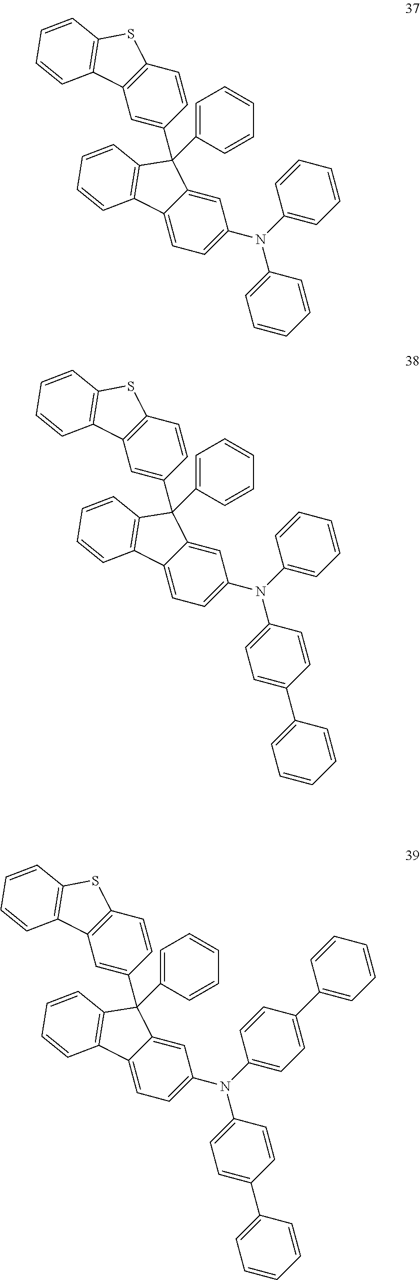 [SMILES: c1ccc(N(c2ccccc2)c2ccc3c(c2)C(c2ccccc2)(c2ccc4sc5ccccc5c4c2)c2ccccc2-3)cc1 ; c1ccc(-c2ccc(N(c3ccccc3)c3ccc4c(c3)C(c3ccccc3)(c3ccc5sc6ccccc6c5c3)c3ccccc3-4)cc2)cc1 ; c1ccc(-c2ccc(N(c3ccc(-c4ccccc4)cc3)c3ccc4c(c3)C(c3ccccc3)(c3ccc5sc6ccccc6c5c3)c3ccccc3-4)cc2)cc1]

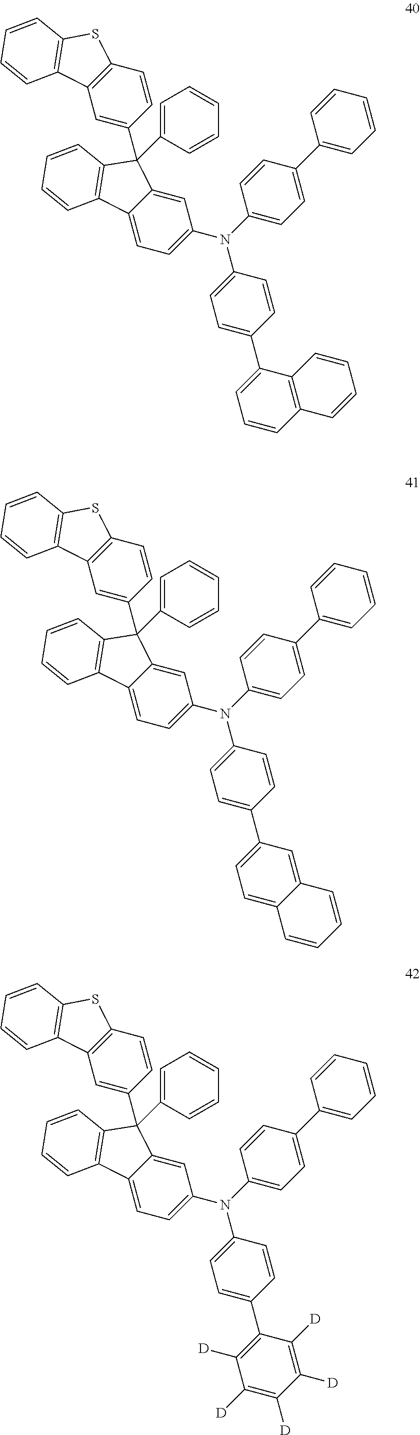 [SMILES: c1ccc(-c2ccc(N(c3ccc(-c4cccc5ccccc45)cc3)c3ccc4c(c3)C(c3ccccc3)(c3ccc5sc6ccccc6c5c3)c3ccccc3-4)cc2)cc1 ; c1ccc(-c2ccc(N(c3ccc(-c4ccc5ccccc5c4)cc3)c3ccc4c(c3)C(c3ccccc3)(c3ccc5sc6ccccc6c5c3)c3ccccc3-4)cc2)cc1 ; [2H]c1c([2H])c([2H])c(-c2ccc(N(c3ccc(-c4ccccc4)cc3)c3ccc4c(c3)C(c3ccccc3)(c3ccc5sc6ccccc6c5c3)c3ccccc3-4)cc2)c([2H])c1[2H]]

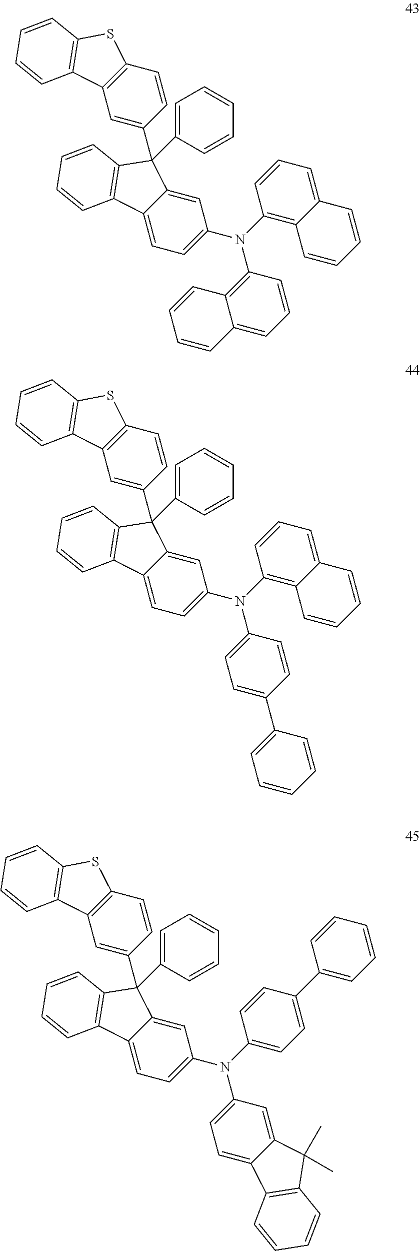 [SMILES: c1ccc(C2(c3ccc4sc5ccccc5c4c3)c3ccccc3-c3ccc(N(c4cccc5ccccc45)c4cccc5ccccc45)cc32)cc1 ; c1ccc(-c2ccc(N(c3ccc4c(c3)C(c3ccccc3)(c3ccc5sc6ccccc6c5c3)c3ccccc3-4)c3cccc4ccccc34)cc2)cc1 ; CC1(C)c2ccccc2-c2ccc(N(c3ccc(-c4ccccc4)cc3)c3ccc4c(c3)C(c3ccccc3)(c3ccc5sc6ccccc6c5c3)c3ccccc3-4)cc21]

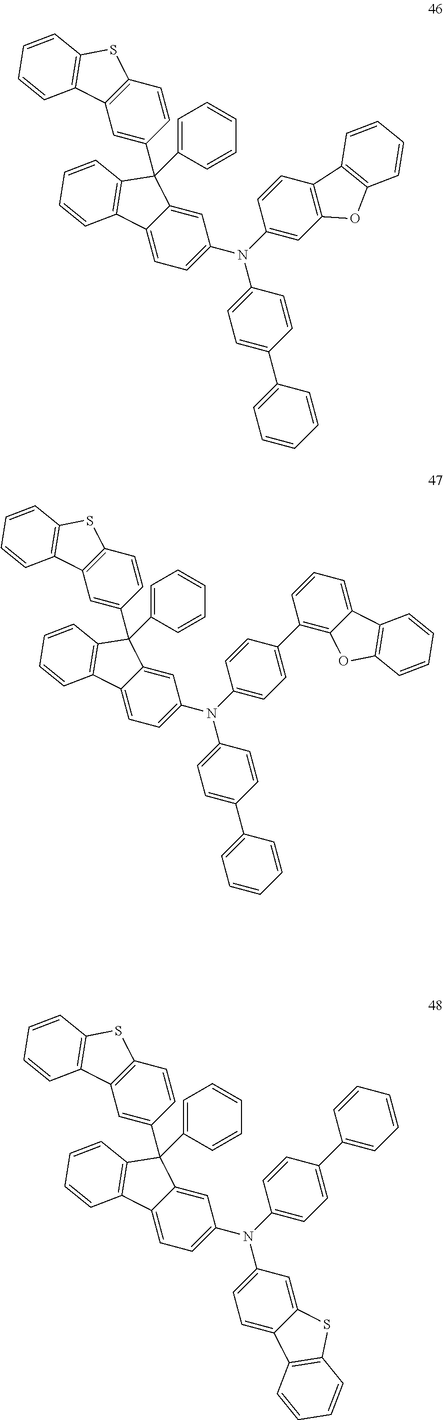 [SMILES: c1ccc(-c2ccc(N(c3ccc4c(c3)C(c3ccccc3)(c3ccc5sc6ccccc6c5c3)c3ccccc3-4)c3ccc4c(c3)oc3ccccc34)cc2)cc1 ; c1ccc(-c2ccc(N(c3ccc(-c4cccc5c4oc4ccccc45)cc3)c3ccc4c(c3)C(c3ccccc3)(c3ccc5sc6ccccc6c5c3)c3ccccc3-4)cc2)cc1 ; c1ccc(-c2ccc(N(c3ccc4c(c3)C(c3ccccc3)(c3ccc5sc6ccccc6c5c3)c3ccccc3-4)c3ccc4c(c3)sc3ccccc34)cc2)cc1]

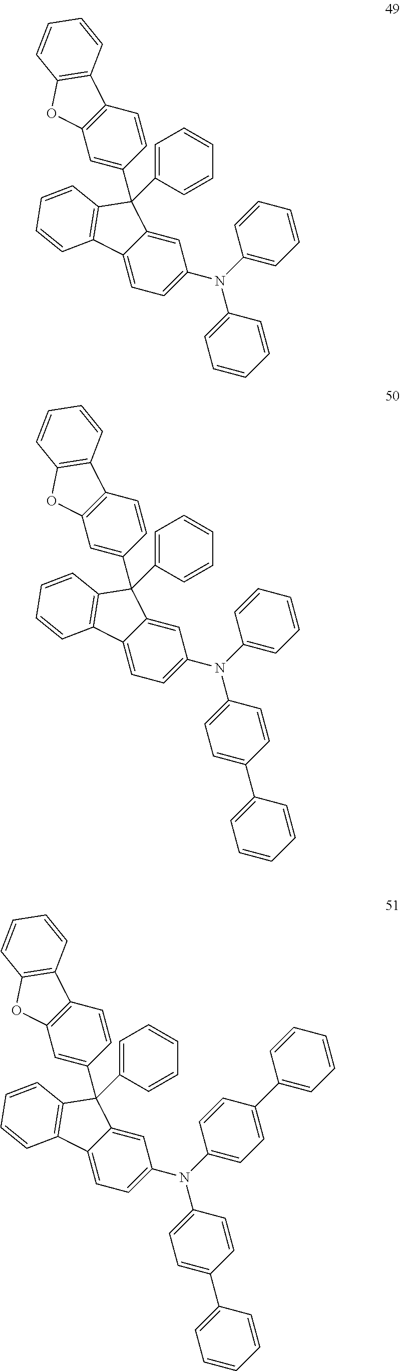 [SMILES: c1ccc(N(c2ccccc2)c2ccc3c(c2)C(c2ccccc2)(c2ccc4c(c2)oc2ccccc24)c2ccccc2-3)cc1 ; c1ccc(-c2ccc(N(c3ccccc3)c3ccc4c(c3)C(c3ccccc3)(c3ccc5c(c3)oc3ccccc35)c3ccccc3-4)cc2)cc1 ; c1ccc(-c2ccc(N(c3ccc(-c4ccccc4)cc3)c3ccc4c(c3)C(c3ccccc3)(c3ccc5c(c3)oc3ccccc35)c3ccccc3-4)cc2)cc1]

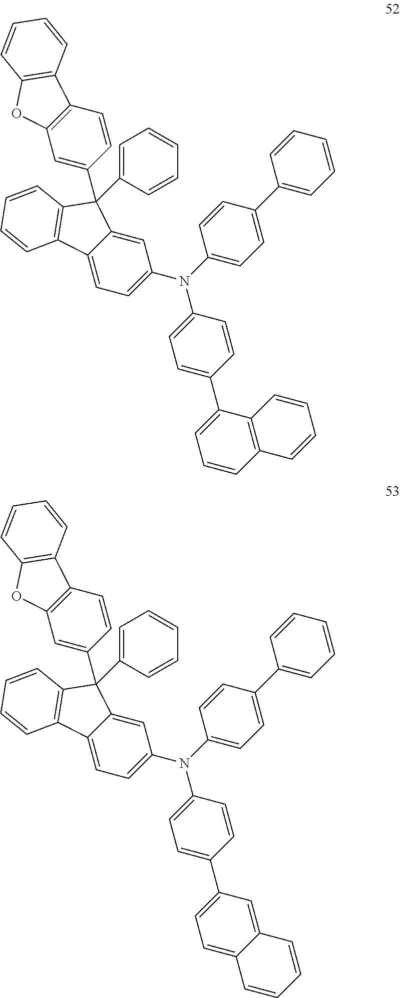 [SMILES: c1ccc(-c2ccc(N(c3ccc(-c4cccc5ccccc45)cc3)c3ccc4c(c3)C(c3ccccc3)(c3ccc5c(c3)oc3ccccc35)c3ccccc3-4)cc2)cc1 ; c1ccc(-c2ccc(N(c3ccc(-c4ccc5ccccc5c4)cc3)c3ccc4c(c3)C(c3ccccc3)(c3ccc5c(c3)oc3ccccc35)c3ccccc3-4)cc2)cc1]

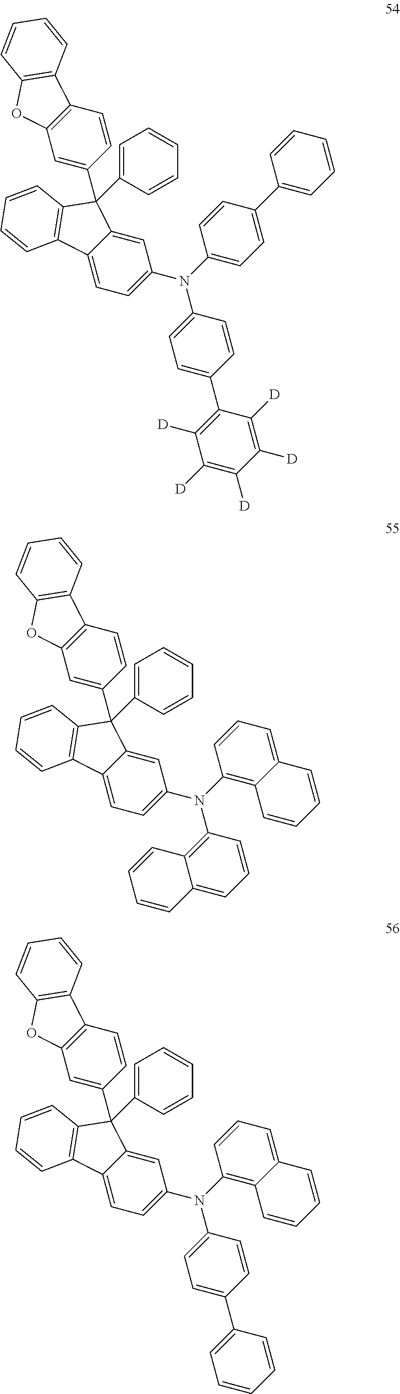 [SMILES: [2H]c1c([2H])c([2H])c(-c2ccc(N(c3ccc(-c4ccccc4)cc3)c3ccc4c(c3)C(c3ccccc3)(c3ccc5c(c3)oc3ccccc35)c3ccccc3-4)cc2)c([2H])c1[2H] ; c1ccc(C2(c3ccc4c(c3)oc3ccccc34)c3ccccc3-c3ccc(N(c4cccc5ccccc45)c4cccc5ccccc45)cc32)cc1 ; c1ccc(-c2ccc(N(c3ccc4c(c3)C(c3ccccc3)(c3ccc5c(c3)oc3ccccc35)c3ccccc3-4)c3cccc4ccccc34)cc2)cc1]

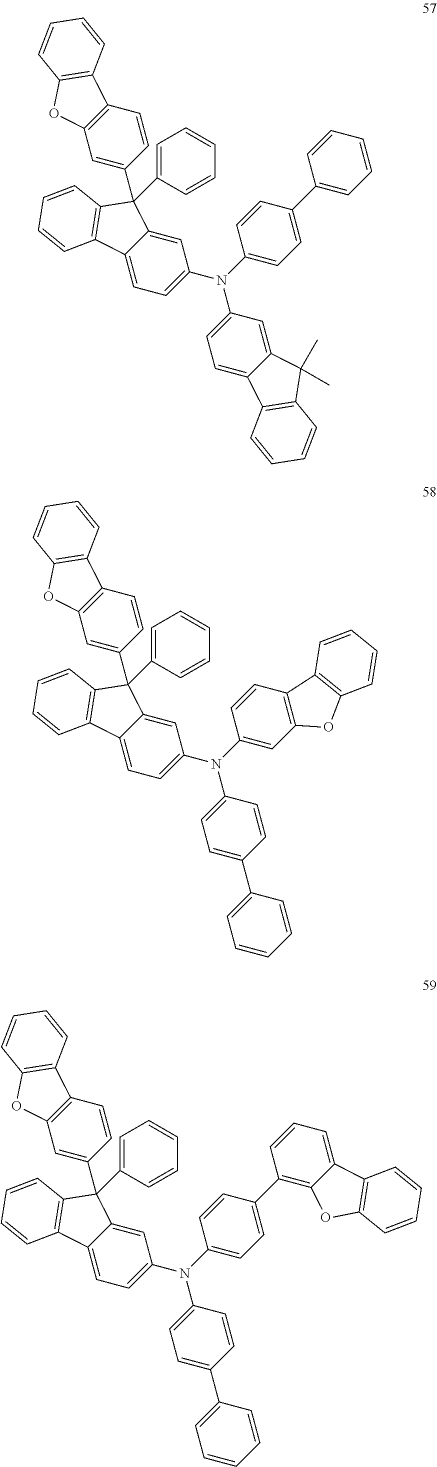 [SMILES: CC1(C)c2ccccc2-c2ccc(N(c3ccc(-c4ccccc4)cc3)c3ccc4c(c3)C(c3ccccc3)(c3ccc5c(c3)oc3ccccc35)c3ccccc3-4)cc21 ; c1ccc(-c2ccc(N(c3ccc4c(c3)C(c3ccccc3)(c3ccc5c(c3)oc3ccccc35)c3ccccc3-4)c3ccc4c(c3)oc3ccccc34)cc2)cc1 ; c1ccc(-c2ccc(N(c3ccc(-c4cccc5c4oc4ccccc45)cc3)c3ccc4c(c3)C(c3ccccc3)(c3ccc5c(c3)oc3ccccc35)c3ccccc3-4)cc2)cc1]

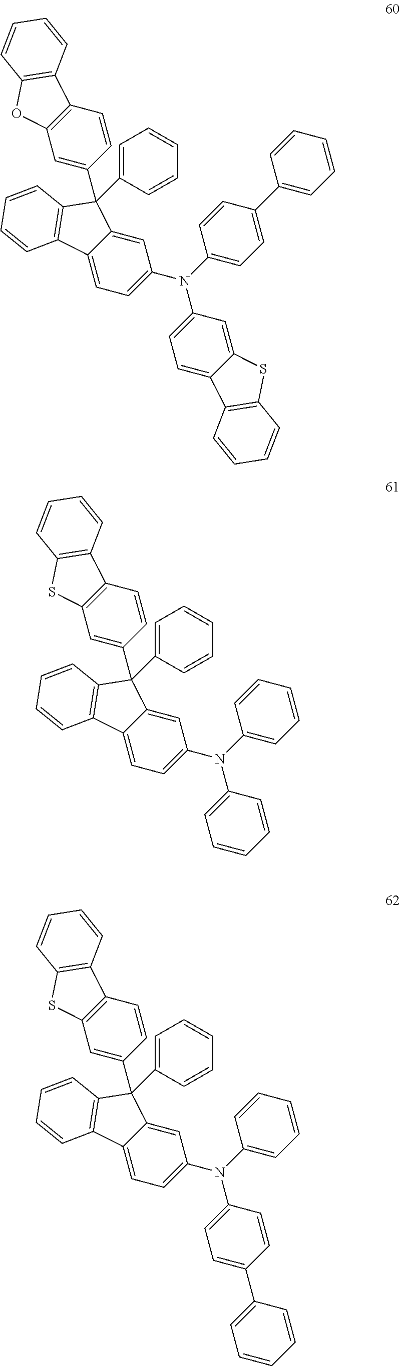 [SMILES: c1ccc(-c2ccc(N(c3ccc4c(c3)C(c3ccccc3)(c3ccc5c(c3)oc3ccccc35)c3ccccc3-4)c3ccc4c(c3)sc3ccccc34)cc2)cc1 ; c1ccc(N(c2ccccc2)c2ccc3c(c2)C(c2ccccc2)(c2ccc4c(c2)sc2ccccc24)c2ccccc2-3)cc1 ; c1ccc(-c2ccc(N(c3ccccc3)c3ccc4c(c3)C(c3ccccc3)(c3ccc5c(c3)sc3ccccc35)c3ccccc3-4)cc2)cc1]

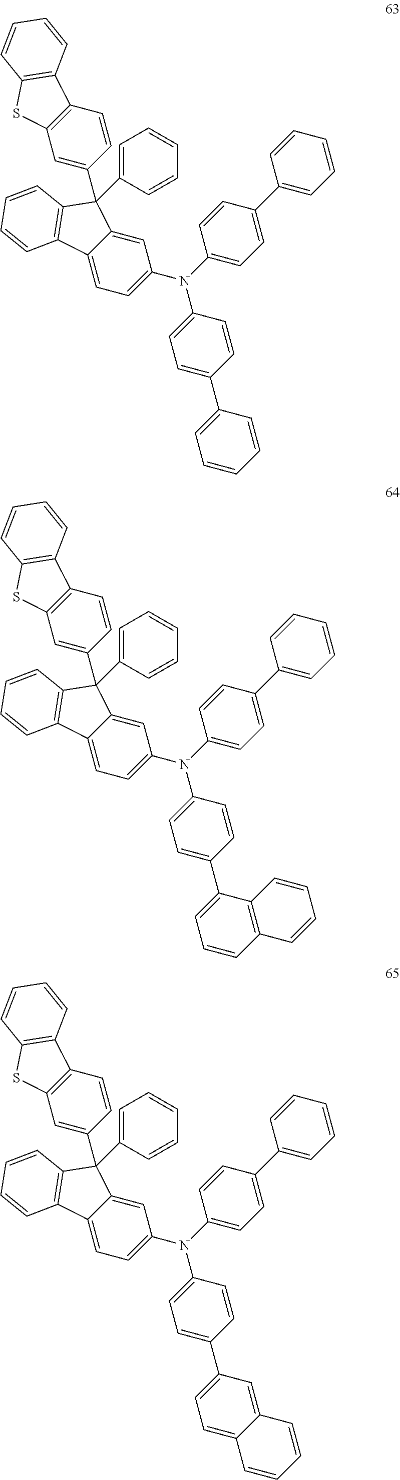 [SMILES: c1ccc(-c2ccc(N(c3ccc(-c4ccccc4)cc3)c3ccc4c(c3)C(c3ccccc3)(c3ccc5c(c3)sc3ccccc35)c3ccccc3-4)cc2)cc1 ; c1ccc(-c2ccc(N(c3ccc(-c4cccc5ccccc45)cc3)c3ccc4c(c3)C(c3ccccc3)(c3ccc5c(c3)sc3ccccc35)c3ccccc3-4)cc2)cc1 ; c1ccc(-c2ccc(N(c3ccc(-c4ccc5ccccc5c4)cc3)c3ccc4c(c3)C(c3ccccc3)(c3ccc5c(c3)sc3ccccc35)c3ccccc3-4)cc2)cc1]

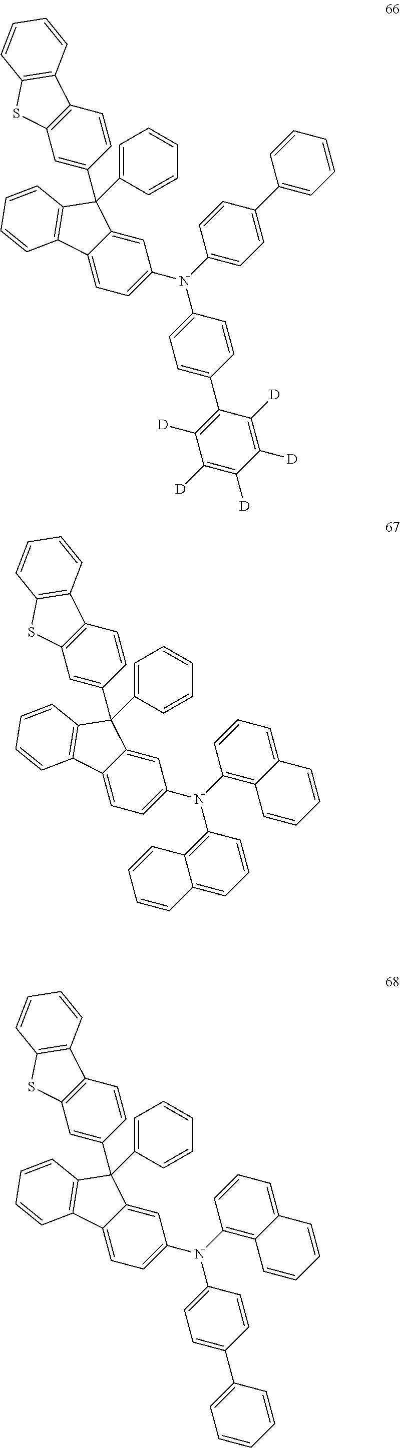 [SMILES: [2H]c1c([2H])c([2H])c(-c2ccc(N(c3ccc(-c4ccccc4)cc3)c3ccc4c(c3)C(c3ccccc3)(c3ccc5c(c3)sc3ccccc35)c3ccccc3-4)cc2)c([2H])c1[2H] ; c1ccc(C2(c3ccc4c(c3)sc3ccccc34)c3ccccc3-c3ccc(N(c4cccc5ccccc45)c4cccc5ccccc45)cc32)cc1 ; c1ccc(-c2ccc(N(c3ccc4c(c3)C(c3ccccc3)(c3ccc5c(c3)sc3ccccc35)c3ccccc3-4)c3cccc4ccccc34)cc2)cc1]

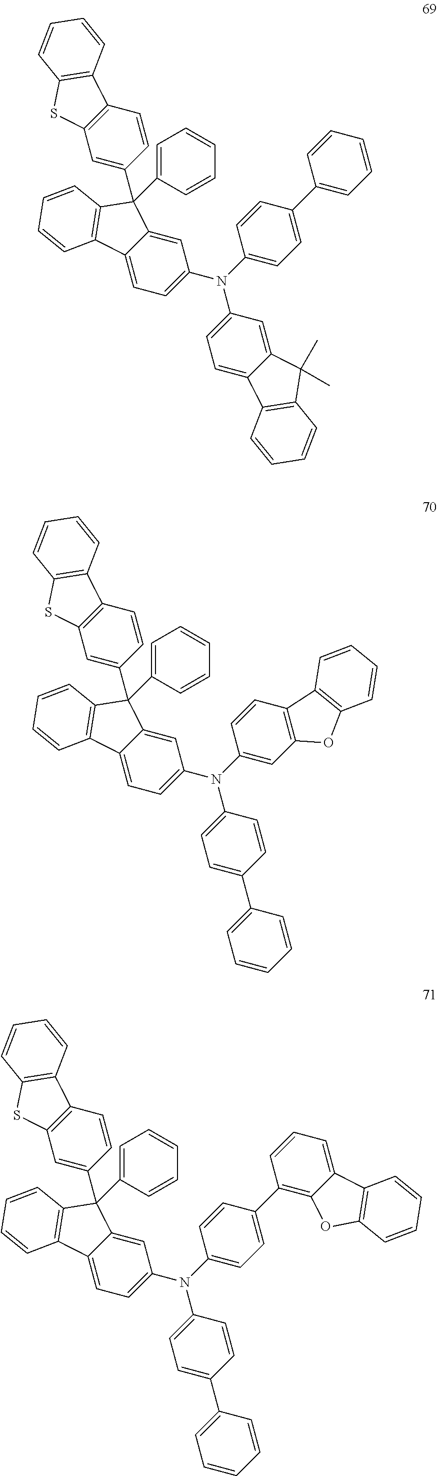 [SMILES: CC1(C)c2ccccc2-c2ccc(N(c3ccc(-c4ccccc4)cc3)c3ccc4c(c3)C(c3ccccc3)(c3ccc5c(c3)sc3ccccc35)c3ccccc3-4)cc21 ; c1ccc(-c2ccc(N(c3ccc4c(c3)C(c3ccccc3)(c3ccc5c(c3)sc3ccccc35)c3ccccc3-4)c3ccc4c(c3)oc3ccccc34)cc2)cc1 ; c1ccc(-c2ccc(N(c3ccc(-c4cccc5c4oc4ccccc45)cc3)c3ccc4c(c3)C(c3ccccc3)(c3ccc5c(c3)sc3ccccc35)c3ccccc3-4)cc2)cc1]

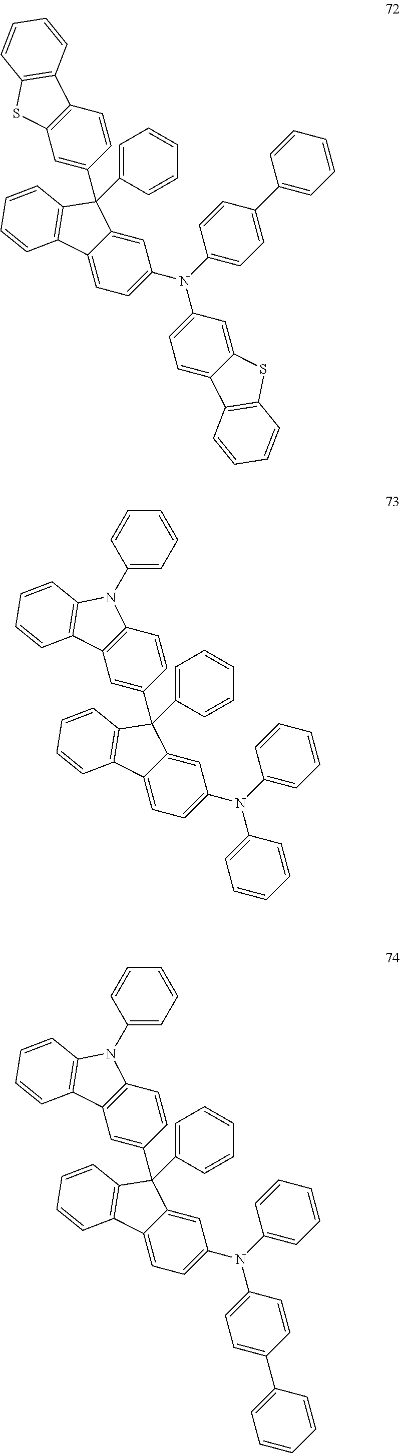 [SMILES: c1ccc(-c2ccc(N(c3ccc4c(c3)C(c3ccccc3)(c3ccc5c(c3)sc3ccccc35)c3ccccc3-4)c3ccc4c(c3)sc3ccccc34)cc2)cc1 ; c1ccc(N(c2ccccc2)c2ccc3c(c2)C(c2ccccc2)(c2ccc4c(c2)c2ccccc2n4-c2ccccc2)c2ccccc2-3)cc1 ; c1ccc(-c2ccc(N(c3ccccc3)c3ccc4c(c3)C(c3ccccc3)(c3ccc5c(c3)c3ccccc3n5-c3ccccc3)c3ccccc3-4)cc2)cc1]

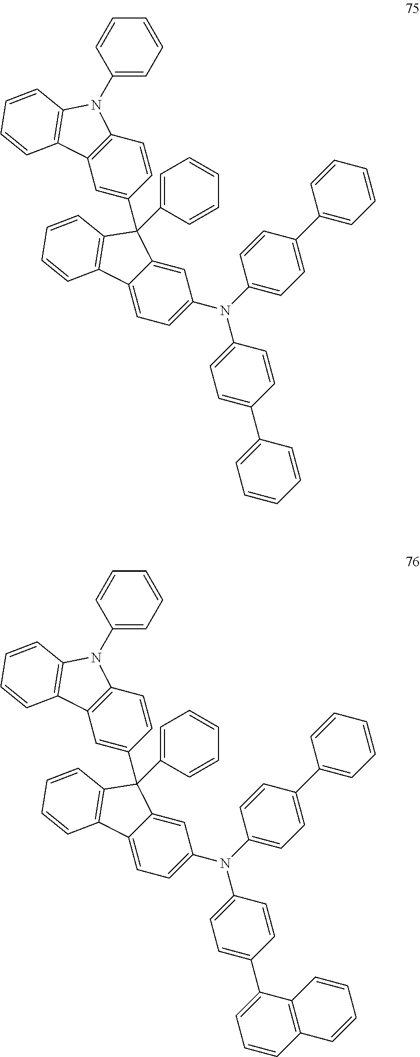 [SMILES: c1ccc(-c2ccc(N(c3ccc(-c4ccccc4)cc3)c3ccc4c(c3)C(c3ccccc3)(c3ccc5c(c3)c3ccccc3n5-c3ccccc3)c3ccccc3-4)cc2)cc1 ; c1ccc(-c2ccc(N(c3ccc(-c4cccc5ccccc45)cc3)c3ccc4c(c3)C(c3ccccc3)(c3ccc5c(c3)c3ccccc3n5-c3ccccc3)c3ccccc3-4)cc2)cc1]

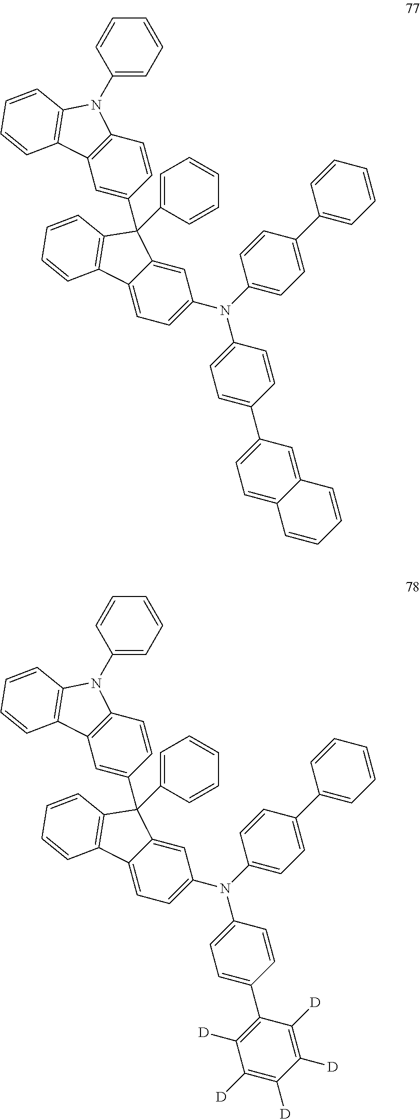 [SMILES: c1ccc(-c2ccc(N(c3ccc(-c4ccc5ccccc5c4)cc3)c3ccc4c(c3)C(c3ccccc3)(c3ccc5c(c3)c3ccccc3n5-c3ccccc3)c3ccccc3-4)cc2)cc1 ; [2H]c1c([2H])c([2H])c(-c2ccc(N(c3ccc(-c4ccccc4)cc3)c3ccc4c(c3)C(c3ccccc3)(c3ccc5c(c3)c3ccccc3n5-c3ccccc3)c3ccccc3-4)cc2)c([2H])c1[2H]]

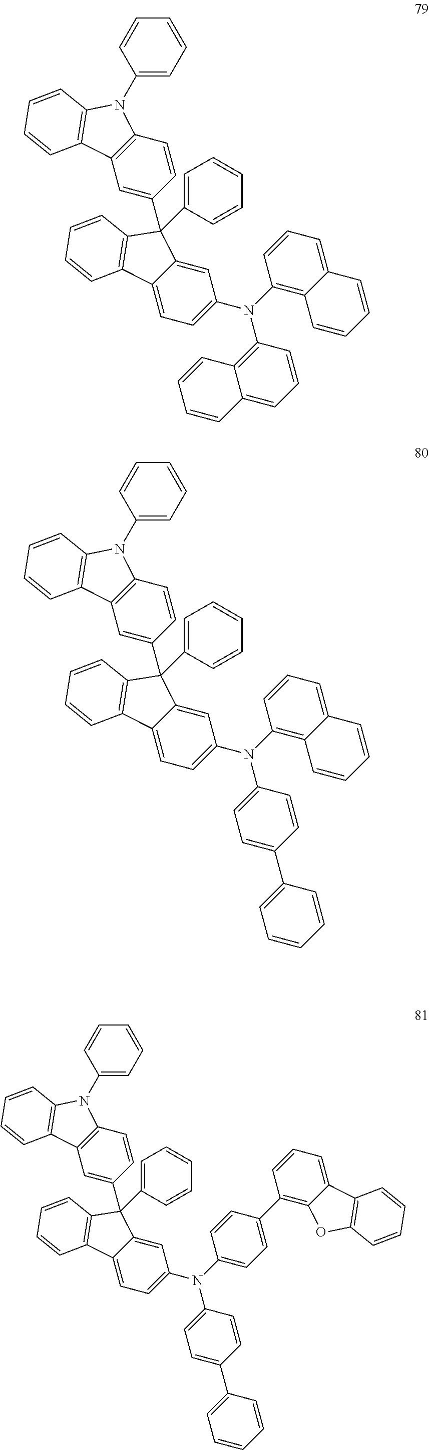 [SMILES: c1ccc(-n2c3ccccc3c3cc(C4(c5ccccc5)c5ccccc5-c5ccc(N(c6cccc7ccccc67)c6cccc7ccccc67)cc54)ccc32)cc1 ; c1ccc(-c2ccc(N(c3ccc4c(c3)C(c3ccccc3)(c3ccc5c(c3)c3ccccc3n5-c3ccccc3)c3ccccc3-4)c3cccc4ccccc34)cc2)cc1 ; c1ccc(-c2ccc(N(c3ccc(-c4cccc5c4oc4ccccc45)cc3)c3ccc4c(c3)C(c3ccccc3)(c3ccc5c(c3)c3ccccc3n5-c3ccccc3)c3ccccc3-4)cc2)cc1]

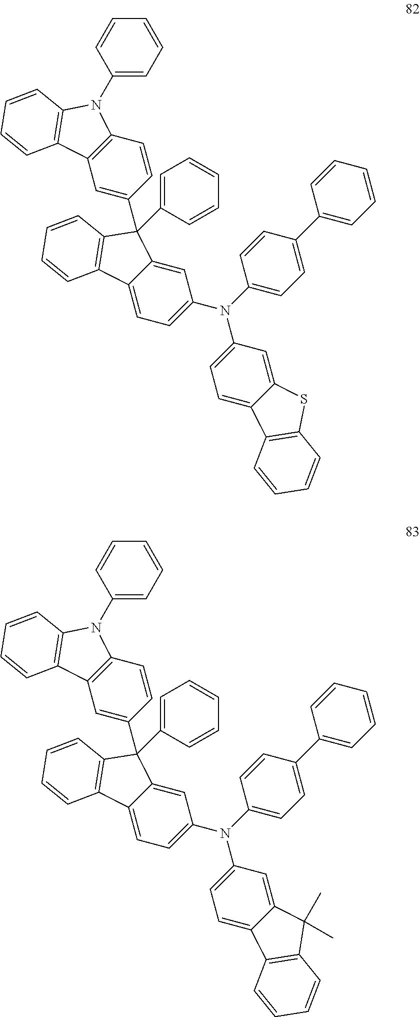 [SMILES: c1ccc(-c2ccc(N(c3ccc4c(c3)C(c3ccccc3)(c3ccc5c(c3)c3ccccc3n5-c3ccccc3)c3ccccc3-4)c3ccc4c(c3)sc3ccccc34)cc2)cc1 ; CC1(C)c2ccccc2-c2ccc(N(c3ccc(-c4ccccc4)cc3)c3ccc4c(c3)C(c3ccccc3)(c3ccc5c(c3)c3ccccc3n5-c3ccccc3)c3ccccc3-4)cc21]

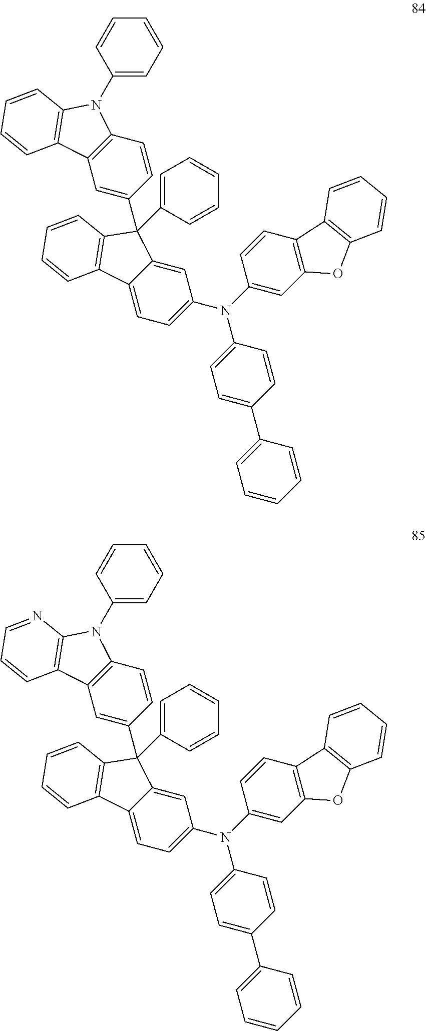 [SMILES: c1ccc(-c2ccc(N(c3ccc4c(c3)C(c3ccccc3)(c3ccc5c(c3)c3ccccc3n5-c3ccccc3)c3ccccc3-4)c3ccc4c(c3)oc3ccccc34)cc2)cc1 ; c1ccc(-c2ccc(N(c3ccc4c(c3)C(c3ccccc3)(c3ccc5c(c3)c3cccnc3n5-c3ccccc3)c3ccccc3-4)c3ccc4c(c3)oc3ccccc34)cc2)cc1]

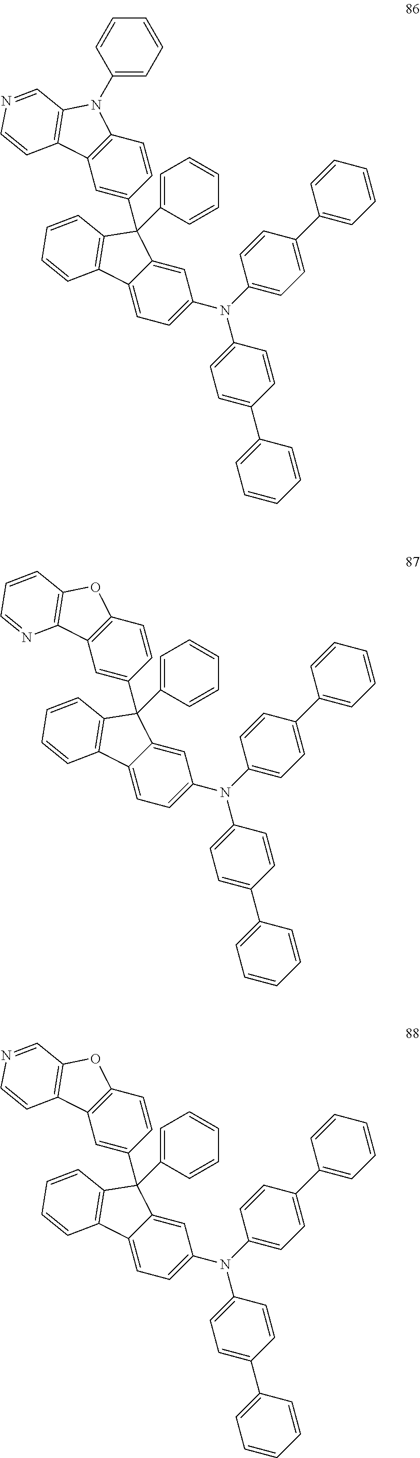 [SMILES: c1ccc(-c2ccc(N(c3ccc(-c4ccccc4)cc3)c3ccc4c(c3)C(c3ccccc3)(c3ccc5c(c3)c3ccncc3n5-c3ccccc3)c3ccccc3-4)cc2)cc1 ; c1ccc(-c2ccc(N(c3ccc(-c4ccccc4)cc3)c3ccc4c(c3)C(c3ccccc3)(c3ccc5oc6cccnc6c5c3)c3ccccc3-4)cc2)cc1 ; c1ccc(-c2ccc(N(c3ccc(-c4ccccc4)cc3)c3ccc4c(c3)C(c3ccccc3)(c3ccc5oc6cnccc6c5c3)c3ccccc3-4)cc2)cc1]

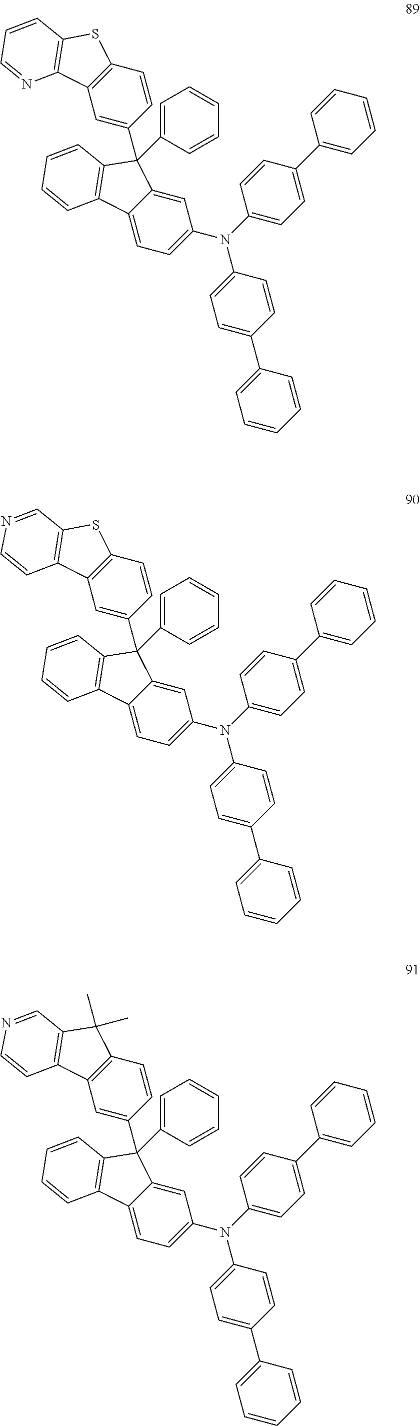 [SMILES: c1ccc(-c2ccc(N(c3ccc(-c4ccccc4)cc3)c3ccc4c(c3)C(c3ccccc3)(c3ccc5sc6cccnc6c5c3)c3ccccc3-4)cc2)cc1 ; c1ccc(-c2ccc(N(c3ccc(-c4ccccc4)cc3)c3ccc4c(c3)C(c3ccccc3)(c3ccc5sc6cnccc6c5c3)c3ccccc3-4)cc2)cc1 ; CC1(C)c2ccc(C3(c4ccccc4)c4ccccc4-c4ccc(N(c5ccc(-c6ccccc6)cc5)c5ccc(-c6ccccc6)cc5)cc43)cc2-c2ccncc21]

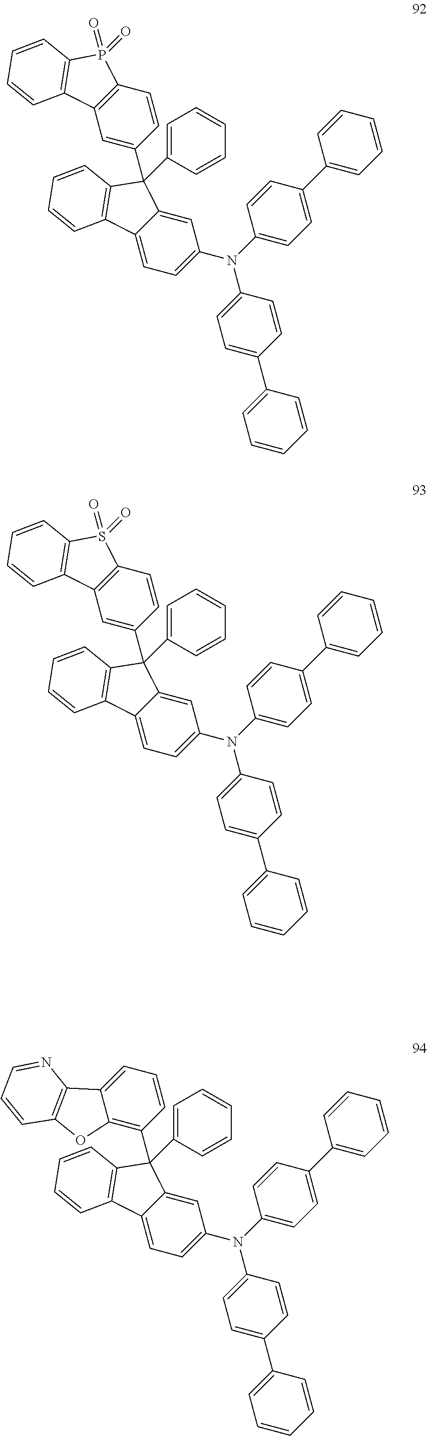

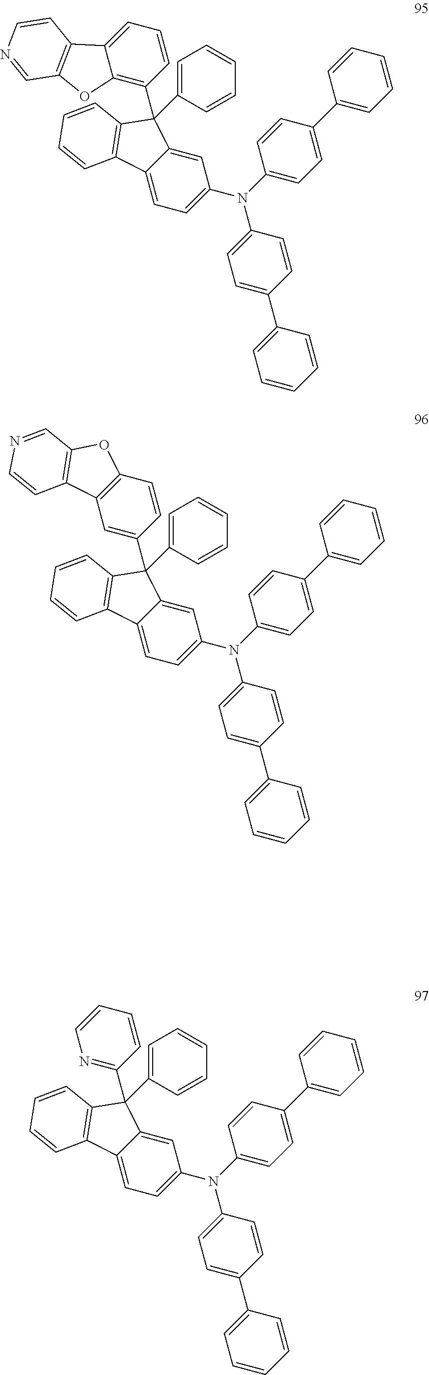 [SMILES: c1ccc(-c2ccc(N(c3ccc(-c4ccccc4)cc3)c3ccc4c(c3)C(c3ccccc3)(c3cccc5c3oc3cnccc35)c3ccccc3-4)cc2)cc1 ; c1ccc(-c2ccc(N(c3ccc(-c4ccccc4)cc3)c3ccc4c(c3)C(c3ccccc3)(c3ccc5oc6cnccc6c5c3)c3ccccc3-4)cc2)cc1 ; c1ccc(-c2ccc(N(c3ccc(-c4ccccc4)cc3)c3ccc4c(c3)C(c3ccccc3)(c3ccccn3)c3ccccc3-4)cc2)cc1]

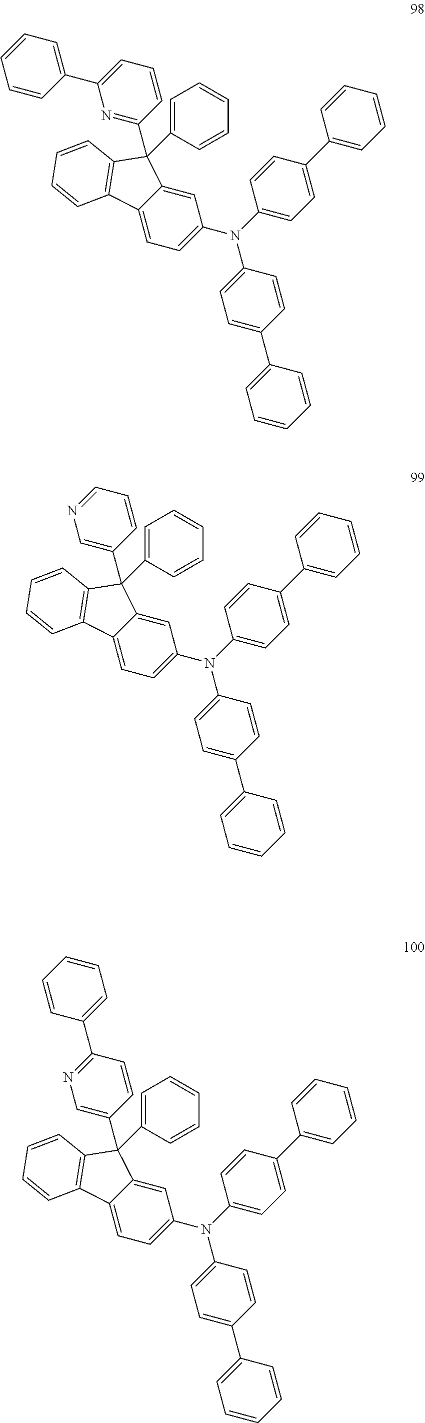 [SMILES: c1ccc(-c2ccc(N(c3ccc(-c4ccccc4)cc3)c3ccc4c(c3)C(c3ccccc3)(c3cccc(-c5ccccc5)n3)c3ccccc3-4)cc2)cc1 ; c1ccc(-c2ccc(N(c3ccc(-c4ccccc4)cc3)c3ccc4c(c3)C(c3ccccc3)(c3cccnc3)c3ccccc3-4)cc2)cc1 ; c1ccc(-c2ccc(N(c3ccc(-c4ccccc4)cc3)c3ccc4c(c3)C(c3ccccc3)(c3ccc(-c5ccccc5)nc3)c3ccccc3-4)cc2)cc1]

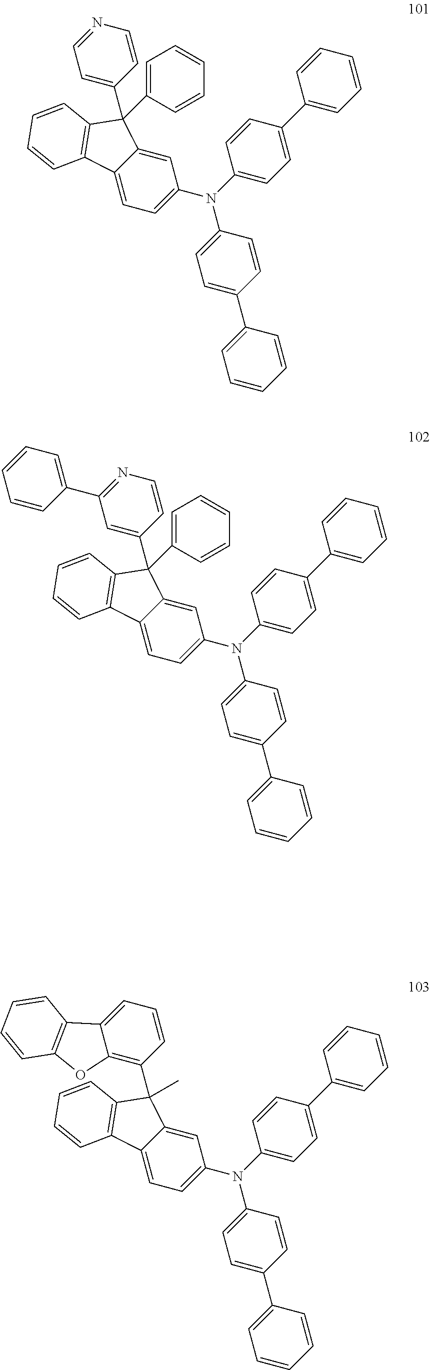 [SMILES: c1ccc(-c2ccc(N(c3ccc(-c4ccccc4)cc3)c3ccc4c(c3)C(c3ccccc3)(c3ccncc3)c3ccccc3-4)cc2)cc1 ; c1ccc(-c2ccc(N(c3ccc(-c4ccccc4)cc3)c3ccc4c(c3)C(c3ccccc3)(c3ccnc(-c5ccccc5)c3)c3ccccc3-4)cc2)cc1 ; CC1(c2cccc3c2oc2ccccc23)c2ccccc2-c2ccc(N(c3ccc(-c4ccccc4)cc3)c3ccc(-c4ccccc4)cc3)cc21]

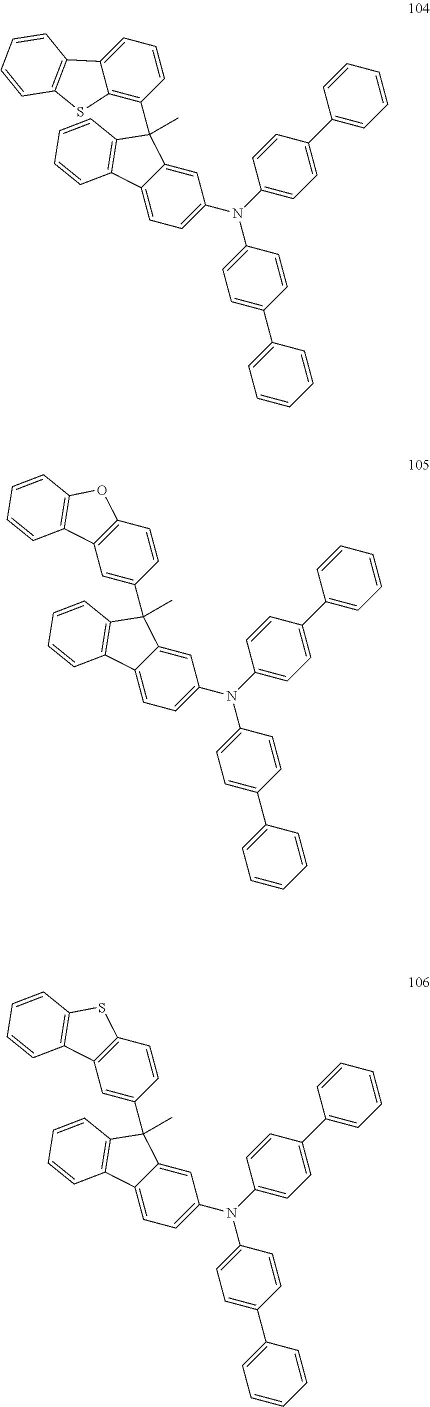 [SMILES: CC1(c2cccc3c2sc2ccccc23)c2ccccc2-c2ccc(N(c3ccc(-c4ccccc4)cc3)c3ccc(-c4ccccc4)cc3)cc21 ; CC1(c2ccc3oc4ccccc4c3c2)c2ccccc2-c2ccc(N(c3ccc(-c4ccccc4)cc3)c3ccc(-c4ccccc4)cc3)cc21 ; CC1(c2ccc3sc4ccccc4c3c2)c2ccccc2-c2ccc(N(c3ccc(-c4ccccc4)cc3)c3ccc(-c4ccccc4)cc3)cc21]

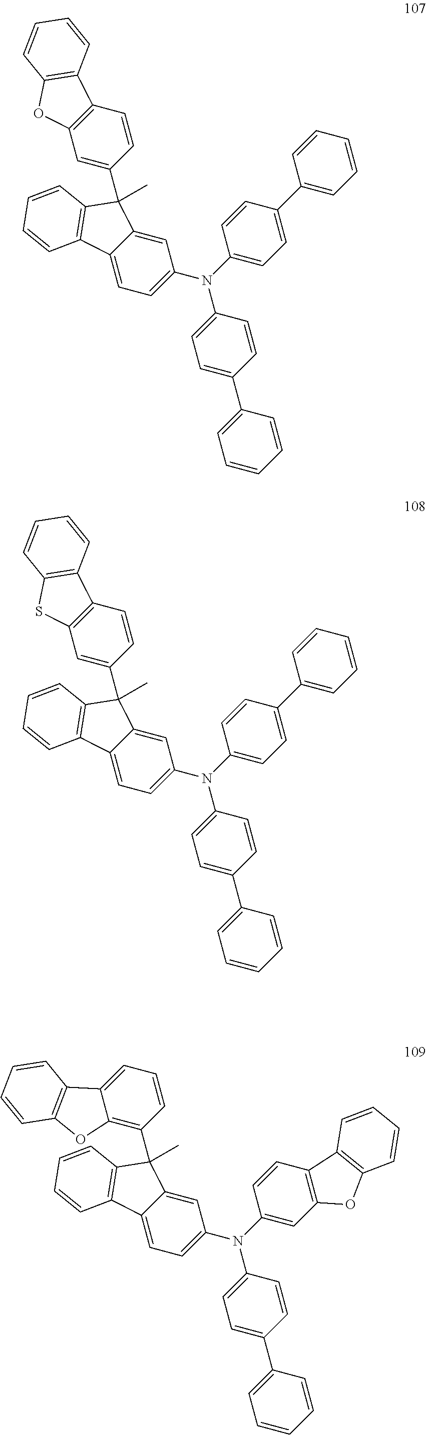 [SMILES: CC1(c2ccc3c(c2)oc2ccccc23)c2ccccc2-c2ccc(N(c3ccc(-c4ccccc4)cc3)c3ccc(-c4ccccc4)cc3)cc21 ; CC1(c2ccc3c(c2)sc2ccccc23)c2ccccc2-c2ccc(N(c3ccc(-c4ccccc4)cc3)c3ccc(-c4ccccc4)cc3)cc21 ; CC1(c2cccc3c2oc2ccccc23)c2ccccc2-c2ccc(N(c3ccc(-c4ccccc4)cc3)c3ccc4c(c3)oc3ccccc34)cc21]

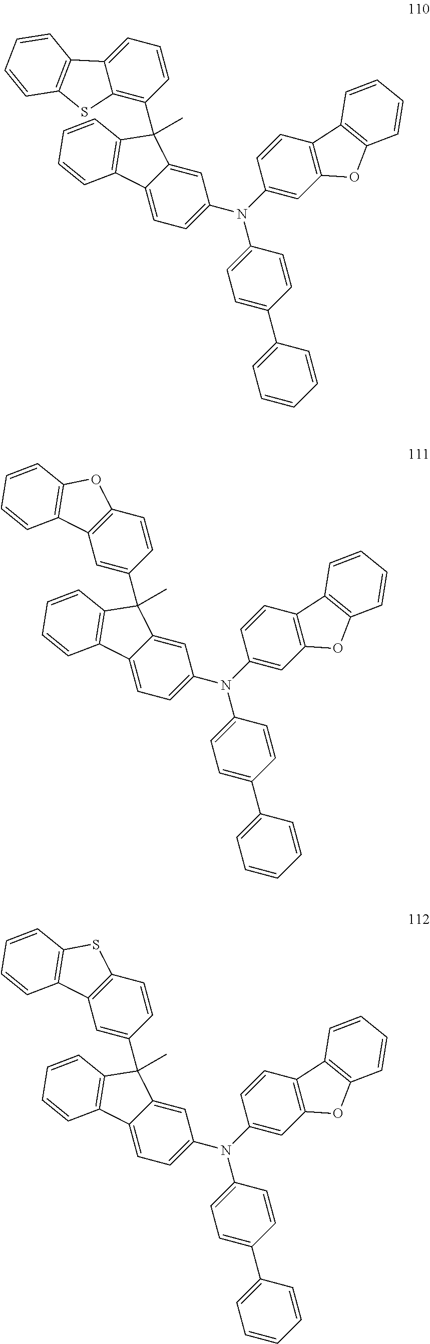 [SMILES: CC1(c2cccc3c2sc2ccccc23)c2ccccc2-c2ccc(N(c3ccc(-c4ccccc4)cc3)c3ccc4c(c3)oc3ccccc34)cc21 ; CC1(c2ccc3oc4ccccc4c3c2)c2ccccc2-c2ccc(N(c3ccc(-c4ccccc4)cc3)c3ccc4c(c3)oc3ccccc34)cc21 ; CC1(c2ccc3sc4ccccc4c3c2)c2ccccc2-c2ccc(N(c3ccc(-c4ccccc4)cc3)c3ccc4c(c3)oc3ccccc34)cc21]

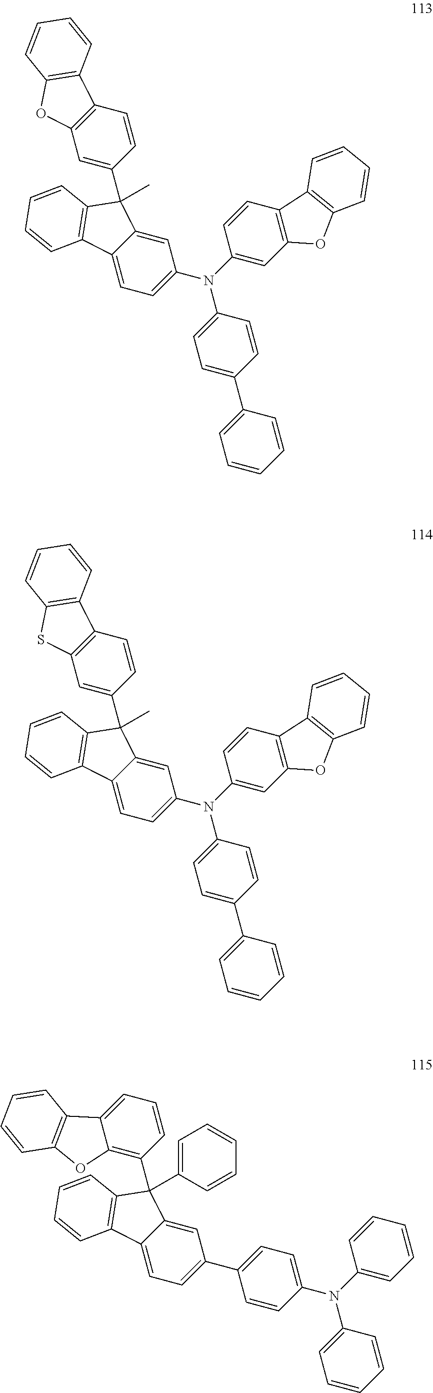 [SMILES: CC1(c2ccc3c(c2)oc2ccccc23)c2ccccc2-c2ccc(N(c3ccc(-c4ccccc4)cc3)c3ccc4c(c3)oc3ccccc34)cc21 ; CC1(c2ccc3c(c2)sc2ccccc23)c2ccccc2-c2ccc(N(c3ccc(-c4ccccc4)cc3)c3ccc4c(c3)oc3ccccc34)cc21 ; c1ccc(N(c2ccccc2)c2ccc(-c3ccc4c(c3)C(c3ccccc3)(c3cccc5c3oc3ccccc35)c3ccccc3-4)cc2)cc1]

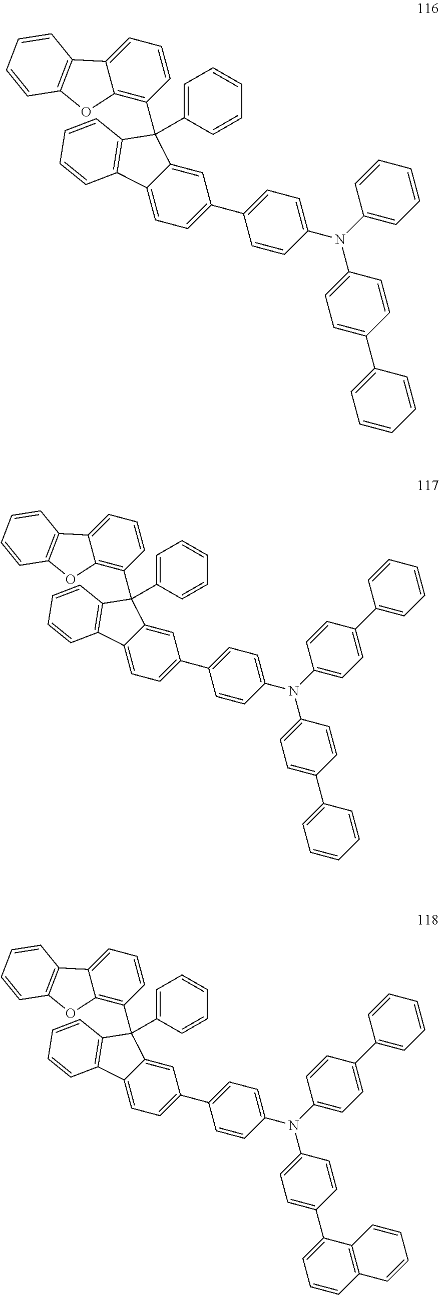 [SMILES: c1ccc(-c2ccc(N(c3ccccc3)c3ccc(-c4ccc5c(c4)C(c4ccccc4)(c4cccc6c4oc4ccccc46)c4ccccc4-5)cc3)cc2)cc1 ; c1ccc(-c2ccc(N(c3ccc(-c4ccccc4)cc3)c3ccc(-c4ccc5c(c4)C(c4ccccc4)(c4cccc6c4oc4ccccc46)c4ccccc4-5)cc3)cc2)cc1 ; c1ccc(-c2ccc(N(c3ccc(-c4ccc5c(c4)C(c4ccccc4)(c4cccc6c4oc4ccccc46)c4ccccc4-5)cc3)c3ccc(-c4cccc5ccccc45)cc3)cc2)cc1]

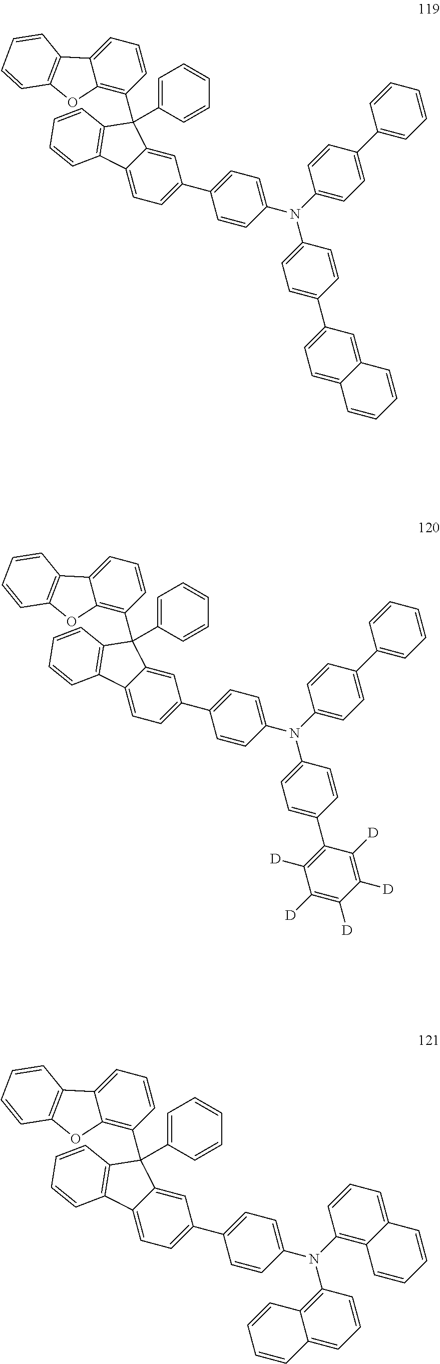 [SMILES: c1ccc(-c2ccc(N(c3ccc(-c4ccc5c(c4)C(c4ccccc4)(c4cccc6c4oc4ccccc46)c4ccccc4-5)cc3)c3ccc(-c4ccc5ccccc5c4)cc3)cc2)cc1 ; [2H]c1c([2H])c([2H])c(-c2ccc(N(c3ccc(-c4ccccc4)cc3)c3ccc(-c4ccc5c(c4)C(c4ccccc4)(c4cccc6c4oc4ccccc46)c4ccccc4-5)cc3)cc2)c([2H])c1[2H] ; c1ccc(C2(c3cccc4c3oc3ccccc34)c3ccccc3-c3ccc(-c4ccc(N(c5cccc6ccccc56)c5cccc6ccccc56)cc4)cc32)cc1]

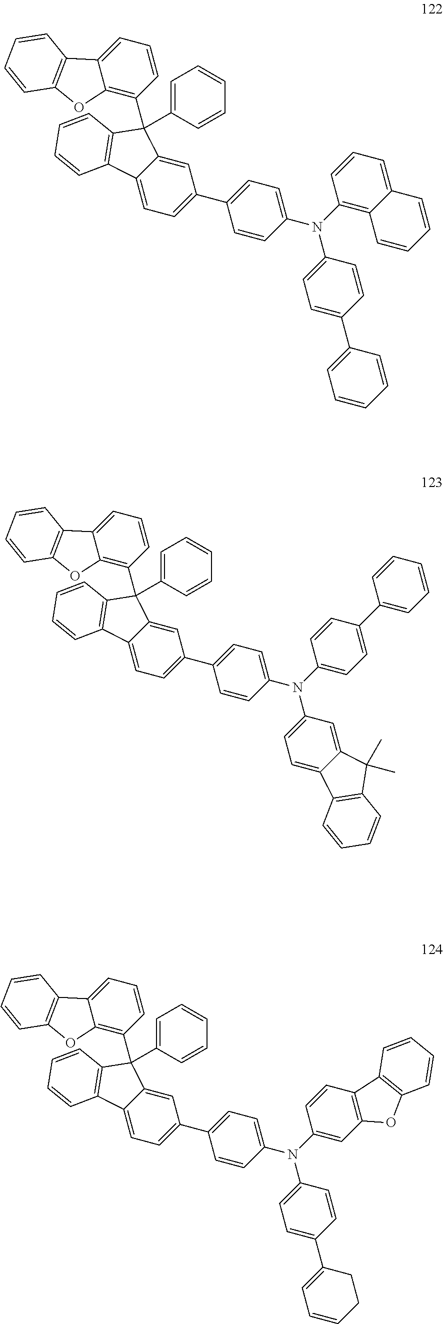 [SMILES: c1ccc(-c2ccc(N(c3ccc(-c4ccc5c(c4)C(c4ccccc4)(c4cccc6c4oc4ccccc46)c4ccccc4-5)cc3)c3cccc4ccccc34)cc2)cc1 ; CC1(C)c2ccccc2-c2ccc(N(c3ccc(-c4ccccc4)cc3)c3ccc(-c4ccc5c(c4)C(c4ccccc4)(c4cccc6c4oc4ccccc46)c4ccccc4-5)cc3)cc21 ; C1=CCCC(c2ccc(N(c3ccc(-c4ccc5c(c4)C(c4ccccc4)(c4cccc6c4oc4ccccc46)c4ccccc4-5)cc3)c3ccc4c(c3)oc3ccccc34)cc2)=C1]

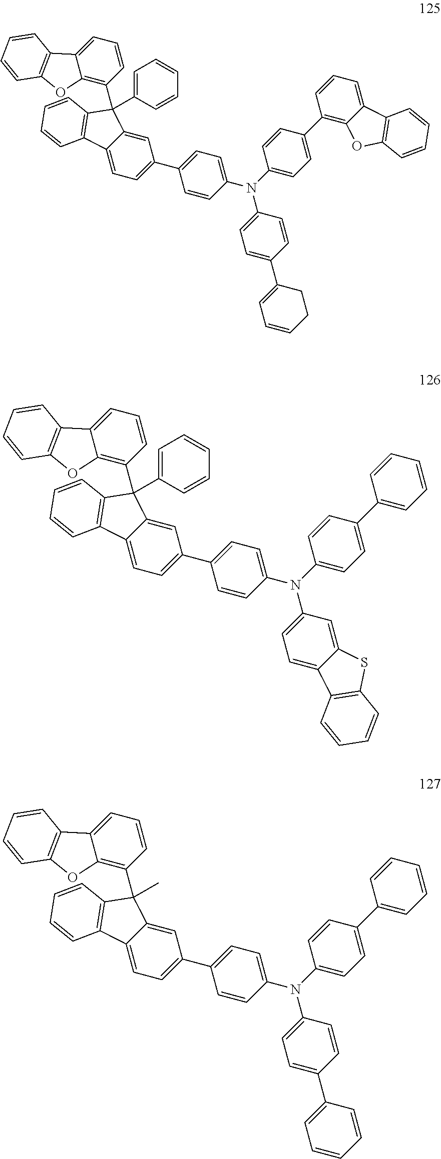 [SMILES: C1=CCCC(c2ccc(N(c3ccc(-c4ccc5c(c4)C(c4ccccc4)(c4cccc6c4oc4ccccc46)c4ccccc4-5)cc3)c3ccc(-c4cccc5c4oc4ccccc45)cc3)cc2)=C1 ; c1ccc(-c2ccc(N(c3ccc(-c4ccc5c(c4)C(c4ccccc4)(c4cccc6c4oc4ccccc46)c4ccccc4-5)cc3)c3ccc4c(c3)sc3ccccc34)cc2)cc1 ; CC1(c2cccc3c2oc2ccccc23)c2ccccc2-c2ccc(-c3ccc(N(c4ccc(-c5ccccc5)cc4)c4ccc(-c5ccccc5)cc4)cc3)cc21]

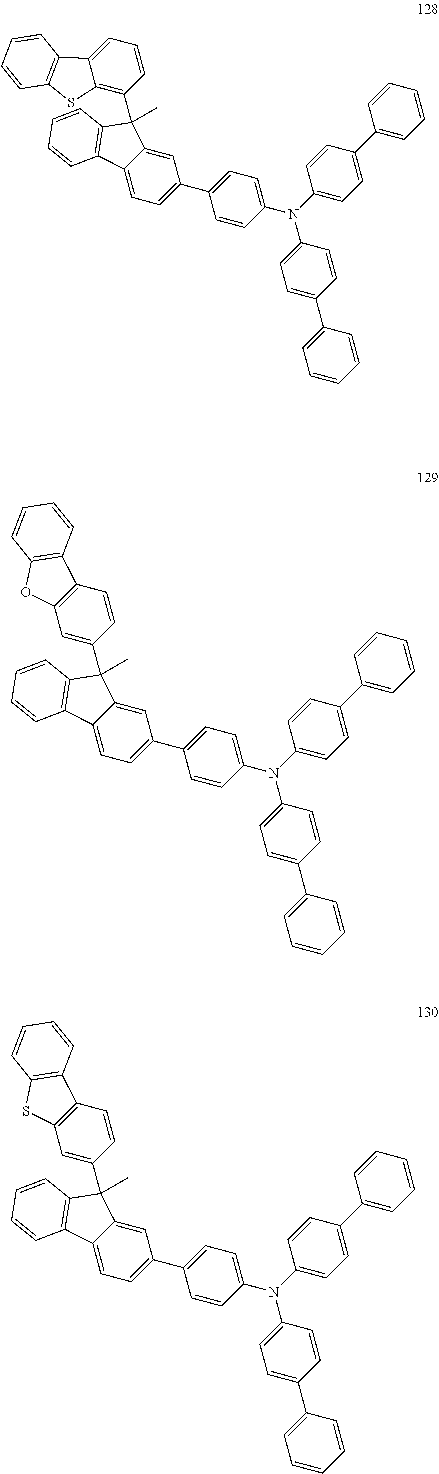 [SMILES: CC1(c2cccc3c2sc2ccccc23)c2ccccc2-c2ccc(-c3ccc(N(c4ccc(-c5ccccc5)cc4)c4ccc(-c5ccccc5)cc4)cc3)cc21 ; CC1(c2ccc3c(c2)oc2ccccc23)c2ccccc2-c2ccc(-c3ccc(N(c4ccc(-c5ccccc5)cc4)c4ccc(-c5ccccc5)cc4)cc3)cc21 ; CC1(c2ccc3c(c2)sc2ccccc23)c2ccccc2-c2ccc(-c3ccc(N(c4ccc(-c5ccccc5)cc4)c4ccc(-c5ccccc5)cc4)cc3)cc21]

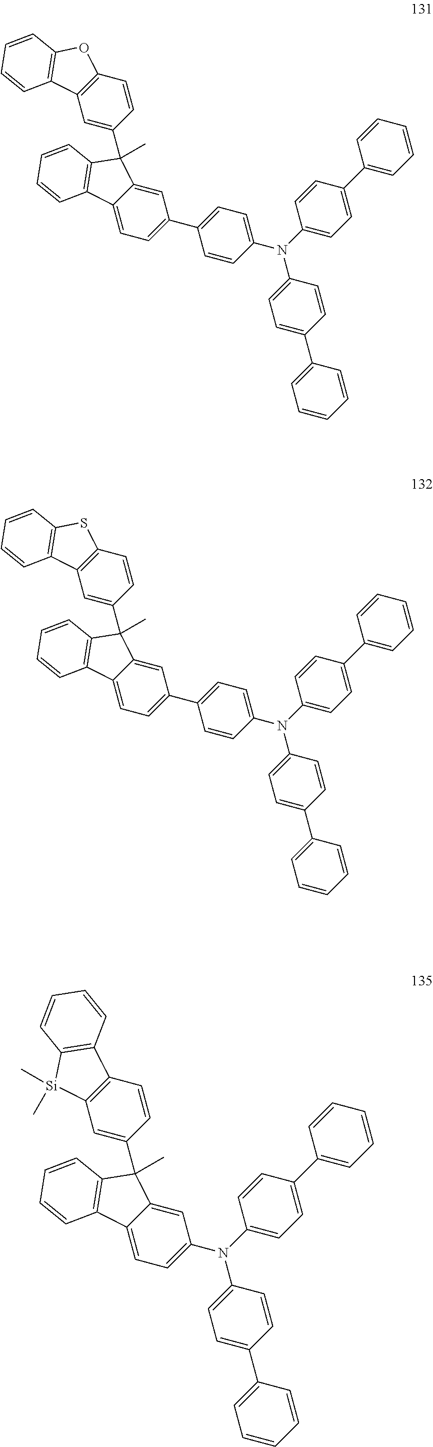 [SMILES: CC1(c2ccc3oc4ccccc4c3c2)c2ccccc2-c2ccc(-c3ccc(N(c4ccc(-c5ccccc5)cc4)c4ccc(-c5ccccc5)cc4)cc3)cc21 ; CC1(c2ccc3sc4ccccc4c3c2)c2ccccc2-c2ccc(-c3ccc(N(c4ccc(-c5ccccc5)cc4)c4ccc(-c5ccccc5)cc4)cc3)cc21 ; CC1(c2ccc3c(c2)[Si](C)(C)c2ccccc2-3)c2ccccc2-c2ccc(N(c3ccc(-c4ccccc4)cc3)c3ccc(-c4ccccc4)cc3)cc21]

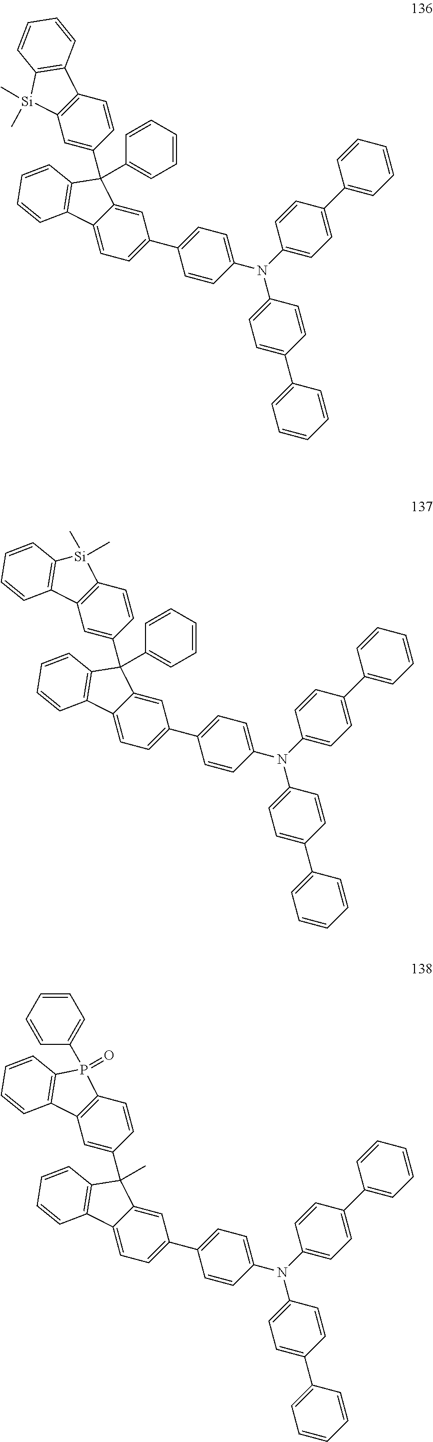 [SMILES: C[Si]1(C)c2ccccc2-c2ccc(C3(c4ccccc4)c4ccccc4-c4ccc(-c5ccc(N(c6ccc(-c7ccccc7)cc6)c6ccc(-c7ccccc7)cc6)cc5)cc43)cc21 ; C[Si]1(C)c2ccccc2-c2cc(C3(c4ccccc4)c4ccccc4-c4ccc(-c5ccc(N(c6ccc(-c7ccccc7)cc6)c6ccc(-c7ccccc7)cc6)cc5)cc43)ccc21 ; CC1(c2ccc3c(c2)-c2ccccc2P3(=O)c2ccccc2)c2ccccc2-c2ccc(-c3ccc(N(c4ccc(-c5ccccc5)cc4)c4ccc(-c5ccccc5)cc4)cc3)cc21]

5. An organic electroluminescent (EL) device comprising a material for an organic EL device represented by Formula 1 in at least one layer: 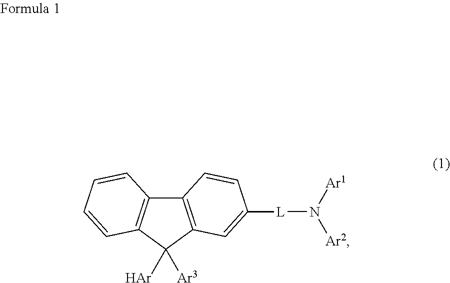
wherein, in Formula 1,
Ar¹ and Ar² are each independently selected from a substituted or unsubstituted aryl group having 6 to 30 carbon atoms for forming a ring, and a substituted or unsubstituted heteroaryl group having 5 to 30 carbon atoms for forming a ring,
Ar³ is selected from a substituted or unsubstituted aryl group having 6 to 30 carbon atoms for forming a ring, a substituted or unsubstituted heteroaryl group having 5 to 30 carbon atoms for forming a ring, and an alkyl group having 1 to 6 carbon atoms,
HAr is a substituted or unsubstituted heteroaryl group having 5 to 30 carbon atoms for forming a ring,
L is selected from a direct linkage, a substituted or unsubstituted arylene group having 6 to 30 carbon atoms for forming a ring, and a substituted or unsubstituted heteroarylene group having 5 to 30 carbon atoms, and
HAr and Ar³ are different from each other.

6. The organic EL device of claim 5, wherein the material for an organic EL device is included in at least one laminated layer between an emission layer and an anode.

7. The organic EL device of claim 5, wherein the material for an organic EL device is included in a hole transport layer.

8. The organic EL device of claim 5, wherein Ar¹ to Ar³ are each independently a substituted or unsubstituted aryl group having 6 to 30 carbon atoms for forming a ring.

9. The organic EL device of claim 5, wherein HAr is selected from a dibenzofuryl group and a dibenzothienyl group.

10. The organic EL device of claim 5, wherein the material for an organic EL device represented by Formula 1 is at least one selected from the following Compounds 1 to 132 and 135 to 138: 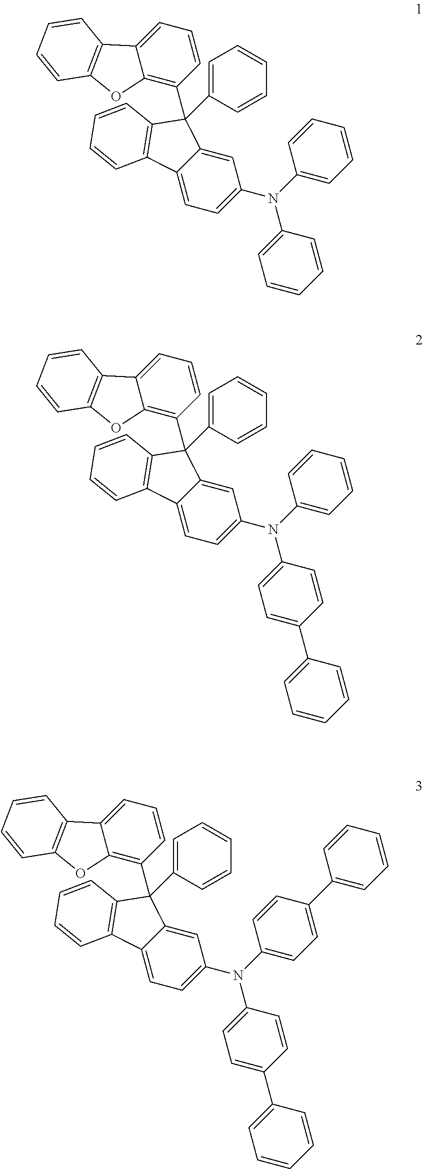 [SMILES: c1ccc(N(c2ccccc2)c2ccc3c(c2)C(c2ccccc2)(c2cccc4c2oc2ccccc24)c2ccccc2-3)cc1 ; c1ccc(-c2ccc(N(c3ccccc3)c3ccc4c(c3)C(c3ccccc3)(c3cccc5c3oc3ccccc35)c3ccccc3-4)cc2)cc1 ; c1ccc(-c2ccc(N(c3ccc(-c4ccccc4)cc3)c3ccc4c(c3)C(c3ccccc3)(c3cccc5c3oc3ccccc35)c3ccccc3-4)cc2)cc1] 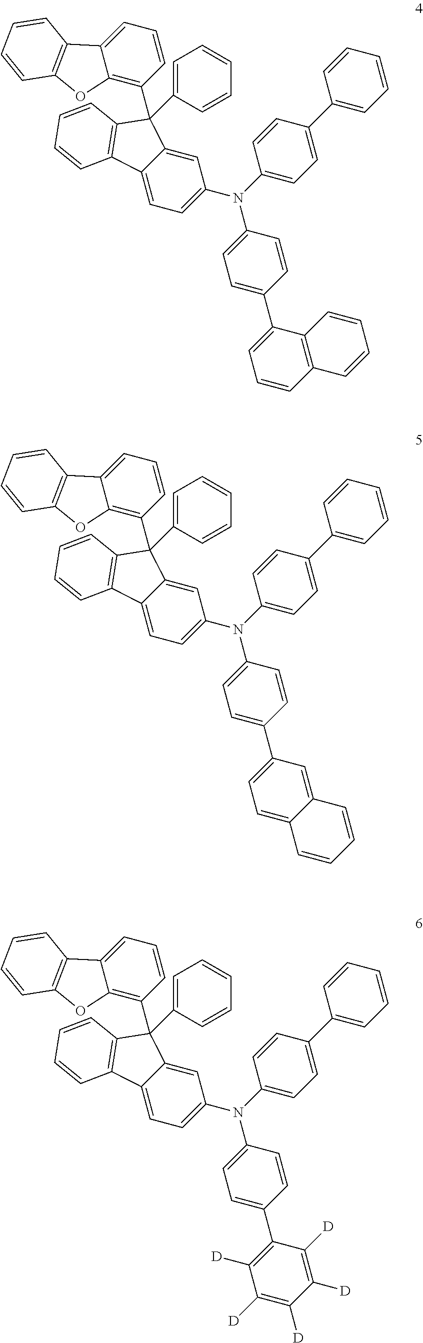 [SMILES: c1ccc(-c2ccc(N(c3ccc(-c4cccc5ccccc45)cc3)c3ccc4c(c3)C(c3ccccc3)(c3cccc5c3oc3ccccc35)c3ccccc3-4)cc2)cc1 ; c1ccc(-c2ccc(N(c3ccc(-c4ccc5ccccc5c4)cc3)c3ccc4c(c3)C(c3ccccc3)(c3cccc5c3oc3ccccc35)c3ccccc3-4)cc2)cc1 ; [2H]c1c([2H])c([2H])c(-c2ccc(N(c3ccc(-c4ccccc4)cc3)c3ccc4c(c3)C(c3ccccc3)(c3cccc5c3oc3ccccc35)c3ccccc3-4)cc2)c([2H])c1[2H]] 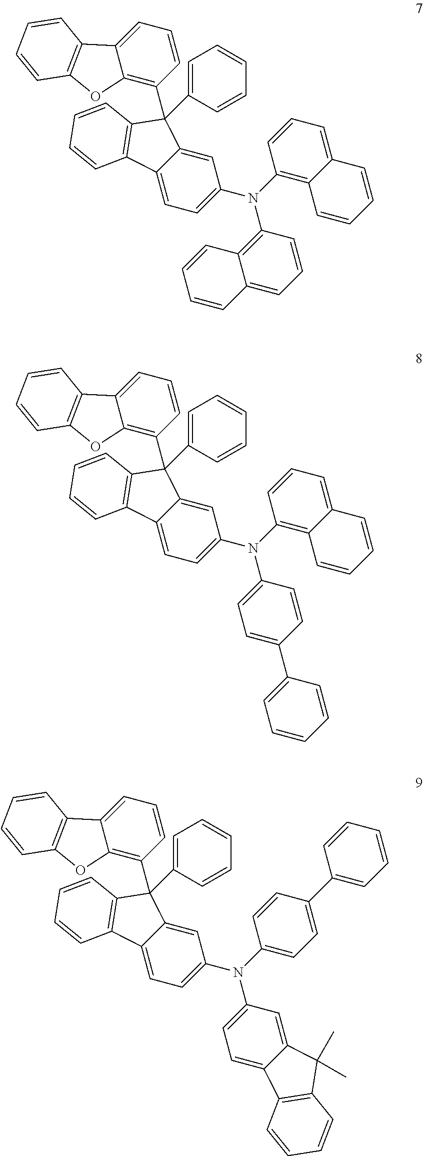 [SMILES: c1ccc(C2(c3cccc4c3oc3ccccc34)c3ccccc3-c3ccc(N(c4cccc5ccccc45)c4cccc5ccccc45)cc32)cc1 ; c1ccc(-c2ccc(N(c3ccc4c(c3)C(c3ccccc3)(c3cccc5c3oc3ccccc35)c3ccccc3-4)c3cccc4ccccc34)cc2)cc1 ; CC1(C)c2ccccc2-c2ccc(N(c3ccc(-c4ccccc4)cc3)c3ccc4c(c3)C(c3ccccc3)(c3cccc5c3oc3ccccc35)c3ccccc3-4)cc21] 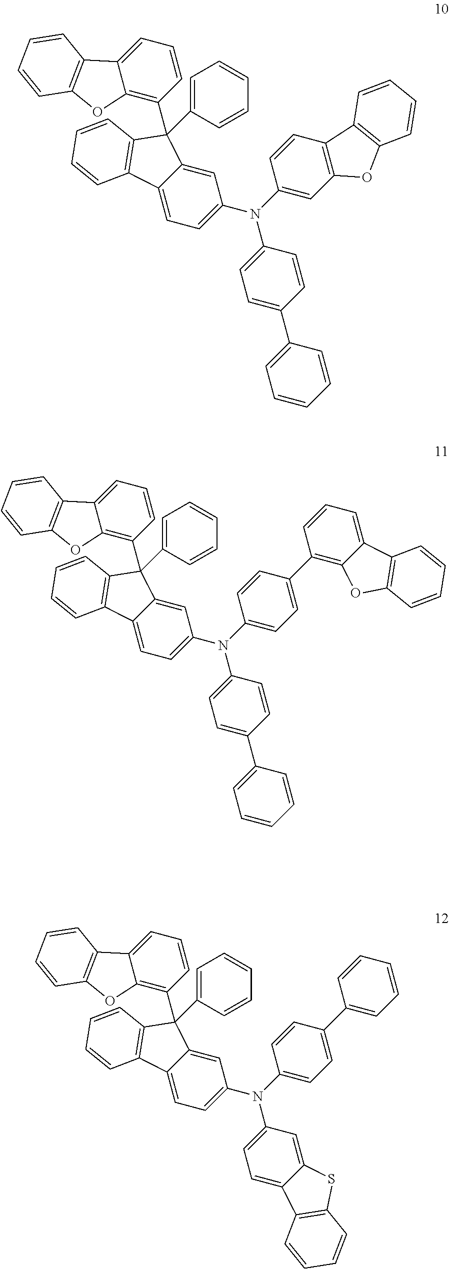 [SMILES: c1ccc(-c2ccc(N(c3ccc4c(c3)C(c3ccccc3)(c3cccc5c3oc3ccccc35)c3ccccc3-4)c3ccc4c(c3)oc3ccccc34)cc2)cc1 ; c1ccc(-c2ccc(N(c3ccc(-c4cccc5c4oc4ccccc45)cc3)c3ccc4c(c3)C(c3ccccc3)(c3cccc5c3oc3ccccc35)c3ccccc3-4)cc2)cc1 ; c1ccc(-c2ccc(N(c3ccc4c(c3)C(c3ccccc3)(c3cccc5c3oc3ccccc35)c3ccccc3-4)c3ccc4c(c3)sc3ccccc34)cc2)cc1] 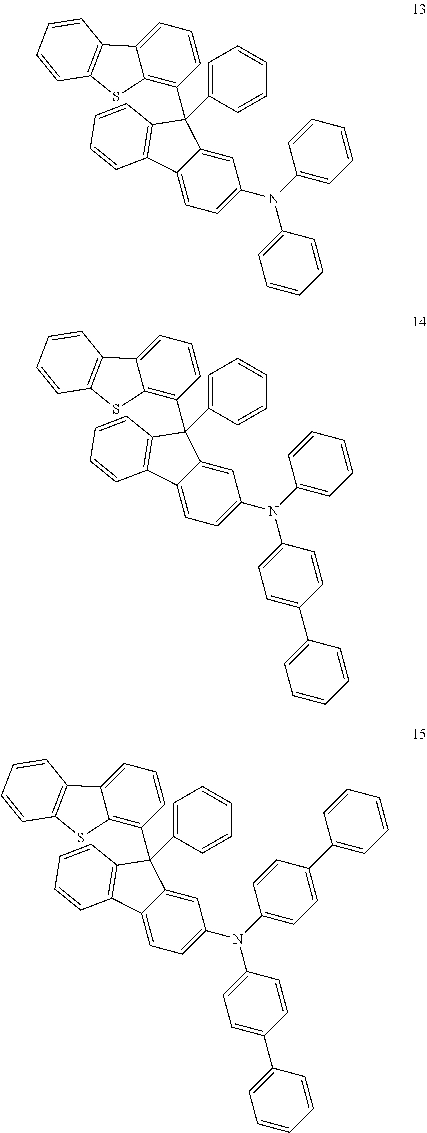 [SMILES: c1ccc(N(c2ccccc2)c2ccc3c(c2)C(c2ccccc2)(c2cccc4c2sc2ccccc24)c2ccccc2-3)cc1 ; c1ccc(-c2ccc(N(c3ccccc3)c3ccc4c(c3)C(c3ccccc3)(c3cccc5c3sc3ccccc35)c3ccccc3-4)cc2)cc1 ; c1ccc(-c2ccc(N(c3ccc(-c4ccccc4)cc3)c3ccc4c(c3)C(c3ccccc3)(c3cccc5c3sc3ccccc35)c3ccccc3-4)cc2)cc1] 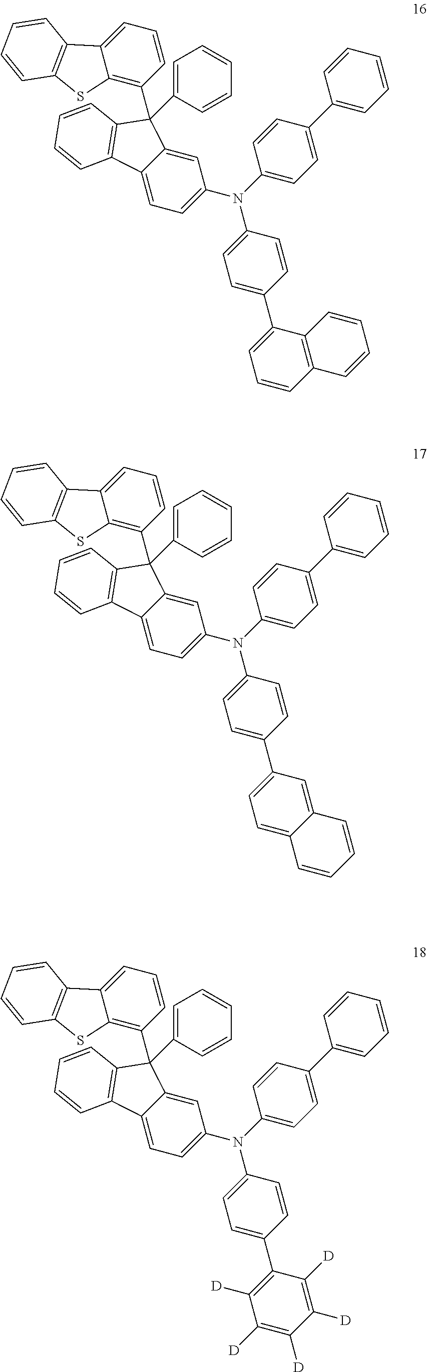 [SMILES: c1ccc(-c2ccc(N(c3ccc(-c4cccc5ccccc45)cc3)c3ccc4c(c3)C(c3ccccc3)(c3cccc5c3sc3ccccc35)c3ccccc3-4)cc2)cc1 ; c1ccc(-c2ccc(N(c3ccc(-c4ccc5ccccc5c4)cc3)c3ccc4c(c3)C(c3ccccc3)(c3cccc5c3sc3ccccc35)c3ccccc3-4)cc2)cc1 ; [2H]c1c([2H])c([2H])c(-c2ccc(N(c3ccc(-c4ccccc4)cc3)c3ccc4c(c3)C(c3ccccc3)(c3cccc5c3sc3ccccc35)c3ccccc3-4)cc2)c([2H])c1[2H]] 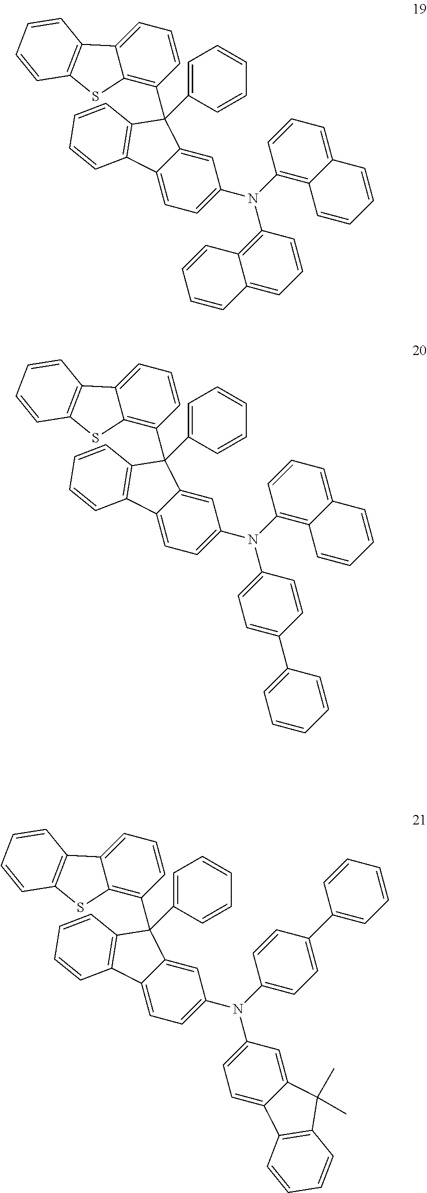 [SMILES: c1ccc(C2(c3cccc4c3sc3ccccc34)c3ccccc3-c3ccc(N(c4cccc5ccccc45)c4cccc5ccccc45)cc32)cc1 ; c1ccc(-c2ccc(N(c3ccc4c(c3)C(c3ccccc3)(c3cccc5c3sc3ccccc35)c3ccccc3-4)c3cccc4ccccc34)cc2)cc1 ; CC1(C)c2ccccc2-c2ccc(N(c3ccc(-c4ccccc4)cc3)c3ccc4c(c3)C(c3ccccc3)(c3cccc5c3sc3ccccc35)c3ccccc3-4)cc21] 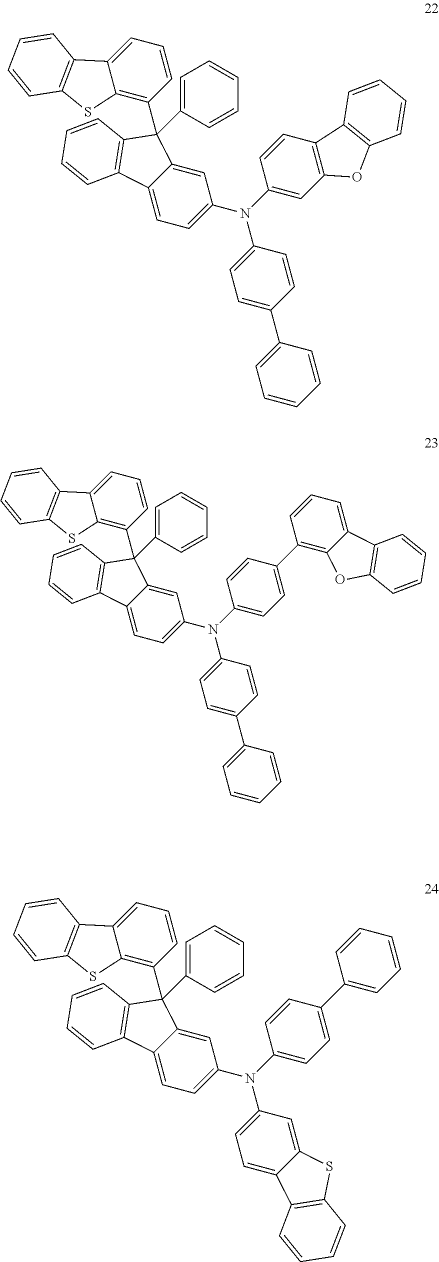 [SMILES: c1ccc(-c2ccc(N(c3ccc4c(c3)C(c3ccccc3)(c3cccc5c3sc3ccccc35)c3ccccc3-4)c3ccc4c(c3)oc3ccccc34)cc2)cc1 ; c1ccc(-c2ccc(N(c3ccc(-c4cccc5c4oc4ccccc45)cc3)c3ccc4c(c3)C(c3ccccc3)(c3cccc5c3sc3ccccc35)c3ccccc3-4)cc2)cc1 ; c1ccc(-c2ccc(N(c3ccc4c(c3)C(c3ccccc3)(c3cccc5c3sc3ccccc35)c3ccccc3-4)c3ccc4c(c3)sc3ccccc34)cc2)cc1] 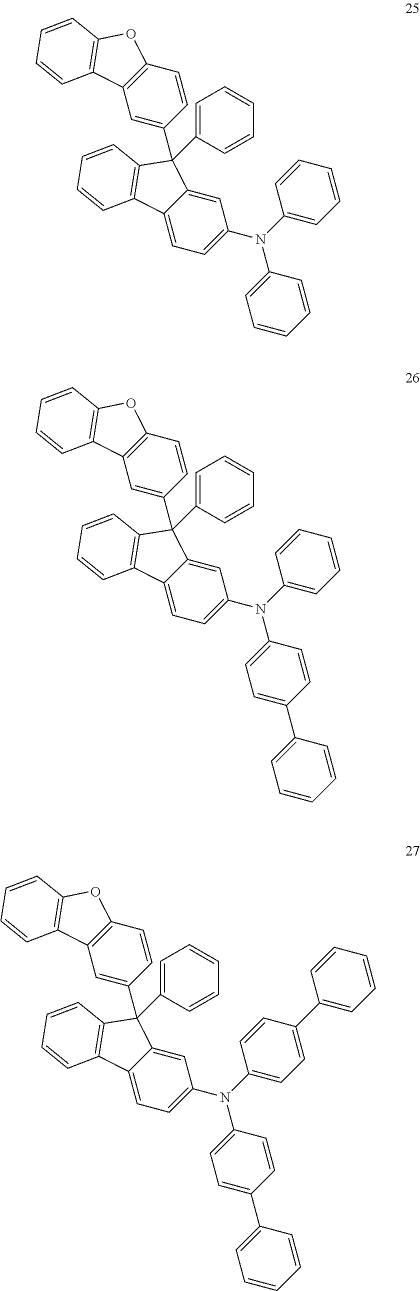 [SMILES: c1ccc(N(c2ccccc2)c2ccc3c(c2)C(c2ccccc2)(c2ccc4oc5ccccc5c4c2)c2ccccc2-3)cc1 ; c1ccc(-c2ccc(N(c3ccccc3)c3ccc4c(c3)C(c3ccccc3)(c3ccc5oc6ccccc6c5c3)c3ccccc3-4)cc2)cc1 ; c1ccc(-c2ccc(N(c3ccc(-c4ccccc4)cc3)c3ccc4c(c3)C(c3ccccc3)(c3ccc5oc6ccccc6c5c3)c3ccccc3-4)cc2)cc1] 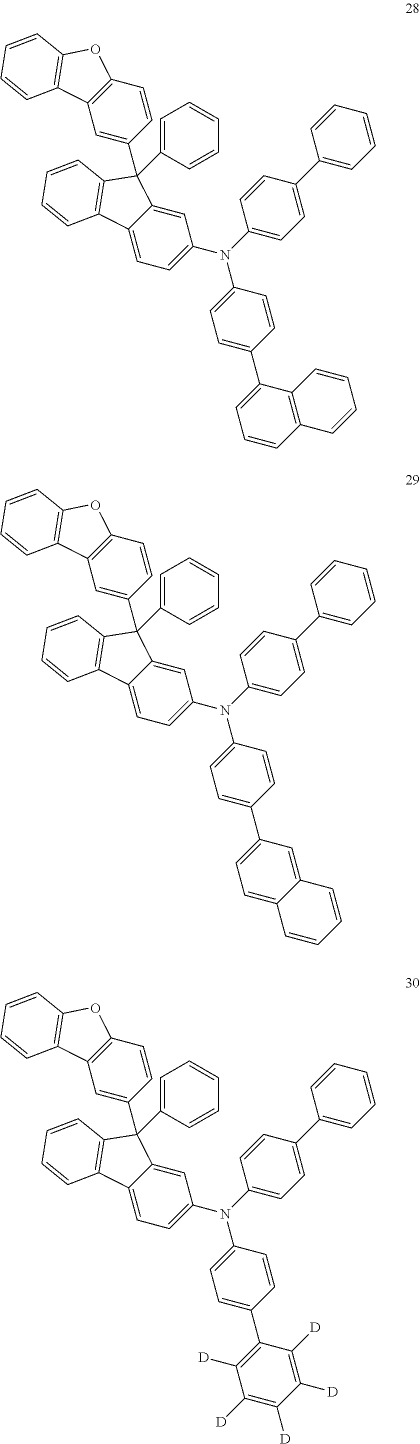 [SMILES: c1ccc(-c2ccc(N(c3ccc(-c4cccc5ccccc45)cc3)c3ccc4c(c3)C(c3ccccc3)(c3ccc5oc6ccccc6c5c3)c3ccccc3-4)cc2)cc1 ; c1ccc(-c2ccc(N(c3ccc(-c4ccc5ccccc5c4)cc3)c3ccc4c(c3)C(c3ccccc3)(c3ccc5oc6ccccc6c5c3)c3ccccc3-4)cc2)cc1 ; [2H]c1c([2H])c([2H])c(-c2ccc(N(c3ccc(-c4ccccc4)cc3)c3ccc4c(c3)C(c3ccccc3)(c3ccc5oc6ccccc6c5c3)c3ccccc3-4)cc2)c([2H])c1[2H]] 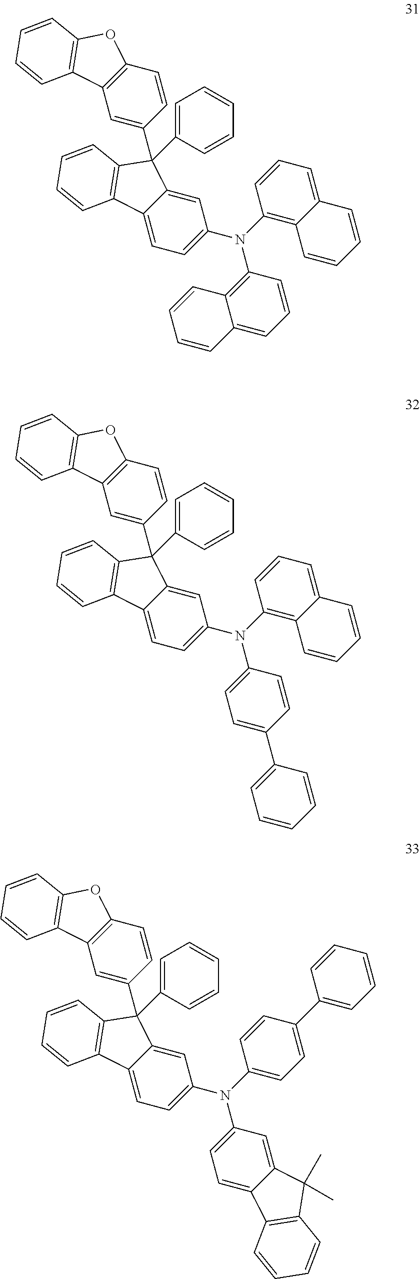 [SMILES: c1ccc(C2(c3ccc4oc5ccccc5c4c3)c3ccccc3-c3ccc(N(c4cccc5ccccc45)c4cccc5ccccc45)cc32)cc1 ; c1ccc(-c2ccc(N(c3ccc4c(c3)C(c3ccccc3)(c3ccc5oc6ccccc6c5c3)c3ccccc3-4)c3cccc4ccccc34)cc2)cc1 ; CC1(C)c2ccccc2-c2ccc(N(c3ccc(-c4ccccc4)cc3)c3ccc4c(c3)C(c3ccccc3)(c3ccc5oc6ccccc6c5c3)c3ccccc3-4)cc21] 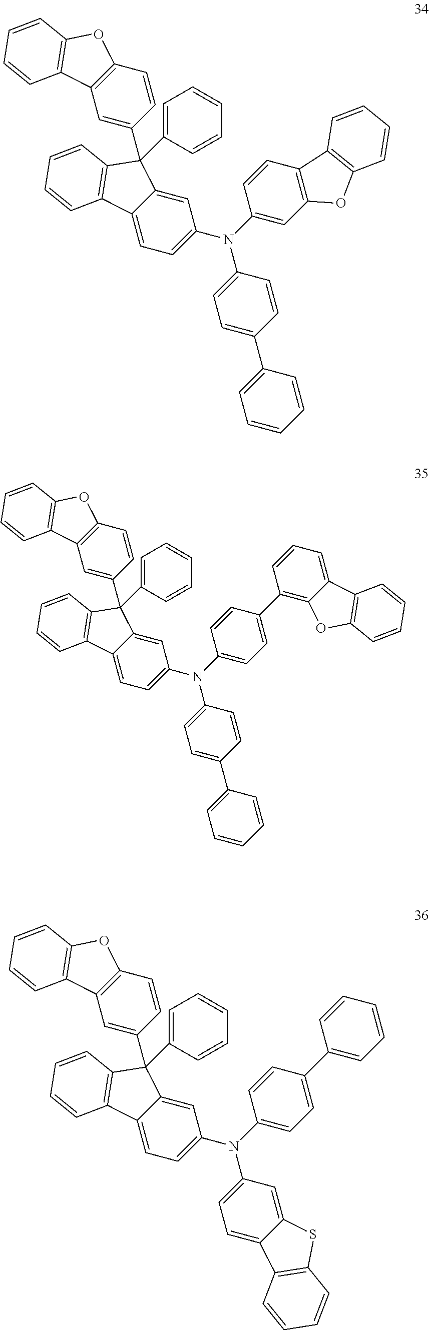 [SMILES: c1ccc(-c2ccc(N(c3ccc4c(c3)C(c3ccccc3)(c3ccc5oc6ccccc6c5c3)c3ccccc3-4)c3ccc4c(c3)oc3ccccc34)cc2)cc1 ; c1ccc(-c2ccc(N(c3ccc(-c4cccc5c4oc4ccccc45)cc3)c3ccc4c(c3)C(c3ccccc3)(c3ccc5oc6ccccc6c5c3)c3ccccc3-4)cc2)cc1 ; c1ccc(-c2ccc(N(c3ccc4c(c3)C(c3ccccc3)(c3ccc5oc6ccccc6c5c3)c3ccccc3-4)c3ccc4c(c3)sc3ccccc34)cc2)cc1] 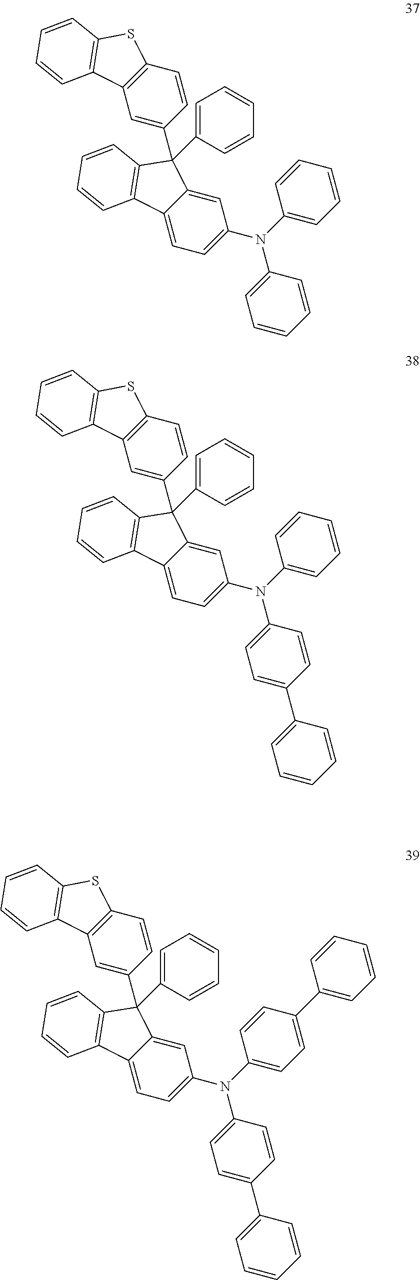 [SMILES: c1ccc(N(c2ccccc2)c2ccc3c(c2)C(c2ccccc2)(c2ccc4sc5ccccc5c4c2)c2ccccc2-3)cc1 ; c1ccc(-c2ccc(N(c3ccccc3)c3ccc4c(c3)C(c3ccccc3)(c3ccc5sc6ccccc6c5c3)c3ccccc3-4)cc2)cc1 ; c1ccc(-c2ccc(N(c3ccc(-c4ccccc4)cc3)c3ccc4c(c3)C(c3ccccc3)(c3ccc5sc6ccccc6c5c3)c3ccccc3-4)cc2)cc1] 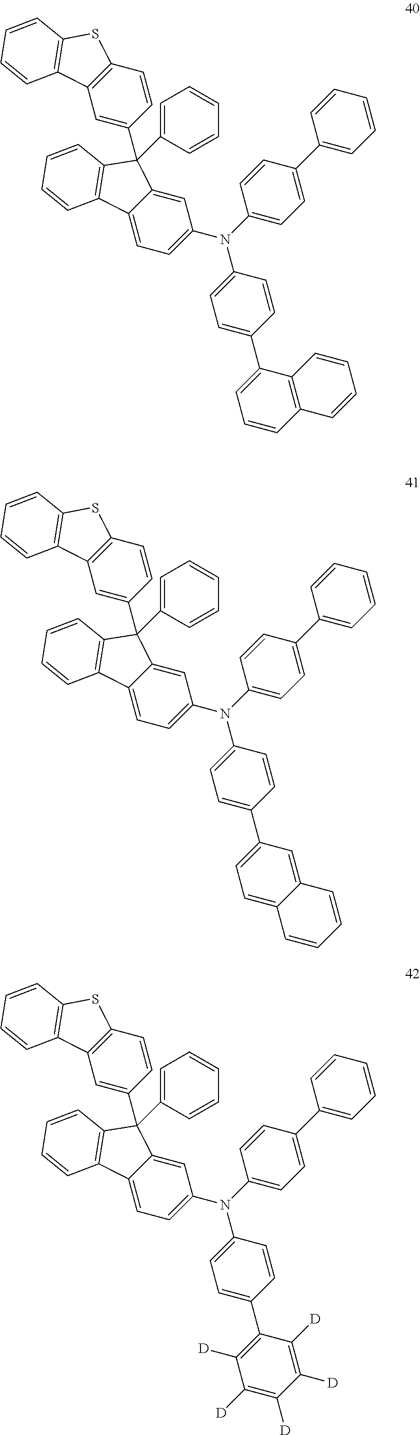 [SMILES: c1ccc(-c2ccc(N(c3ccc(-c4cccc5ccccc45)cc3)c3ccc4c(c3)C(c3ccccc3)(c3ccc5sc6ccccc6c5c3)c3ccccc3-4)cc2)cc1 ; c1ccc(-c2ccc(N(c3ccc(-c4ccc5ccccc5c4)cc3)c3ccc4c(c3)C(c3ccccc3)(c3ccc5sc6ccccc6c5c3)c3ccccc3-4)cc2)cc1 ; [2H]c1c([2H])c([2H])c(-c2ccc(N(c3ccc(-c4ccccc4)cc3)c3ccc4c(c3)C(c3ccccc3)(c3ccc5sc6ccccc6c5c3)c3ccccc3-4)cc2)c([2H])c1[2H]] 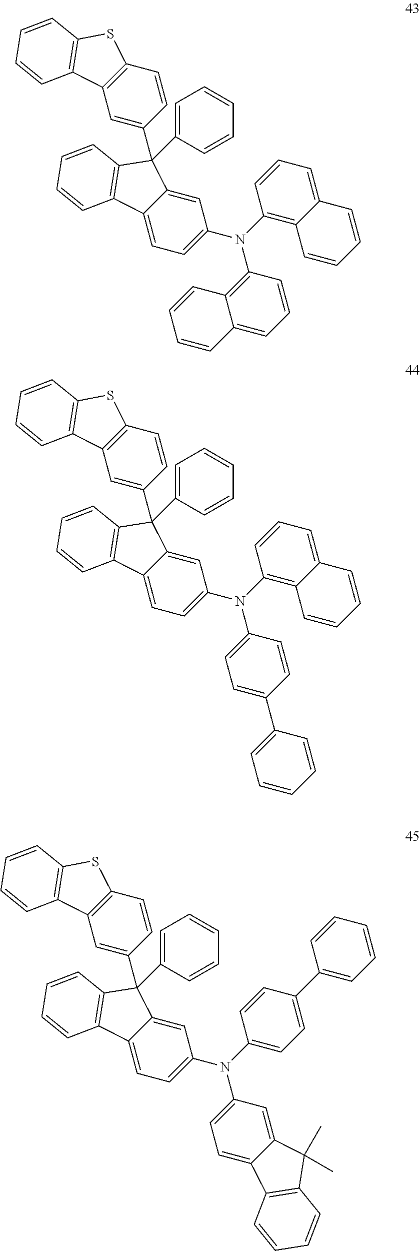 [SMILES: c1ccc(C2(c3ccc4sc5ccccc5c4c3)c3ccccc3-c3ccc(N(c4cccc5ccccc45)c4cccc5ccccc45)cc32)cc1 ; c1ccc(-c2ccc(N(c3ccc4c(c3)C(c3ccccc3)(c3ccc5sc6ccccc6c5c3)c3ccccc3-4)c3cccc4ccccc34)cc2)cc1 ; CC1(C)c2ccccc2-c2ccc(N(c3ccc(-c4ccccc4)cc3)c3ccc4c(c3)C(c3ccccc3)(c3ccc5sc6ccccc6c5c3)c3ccccc3-4)cc21] 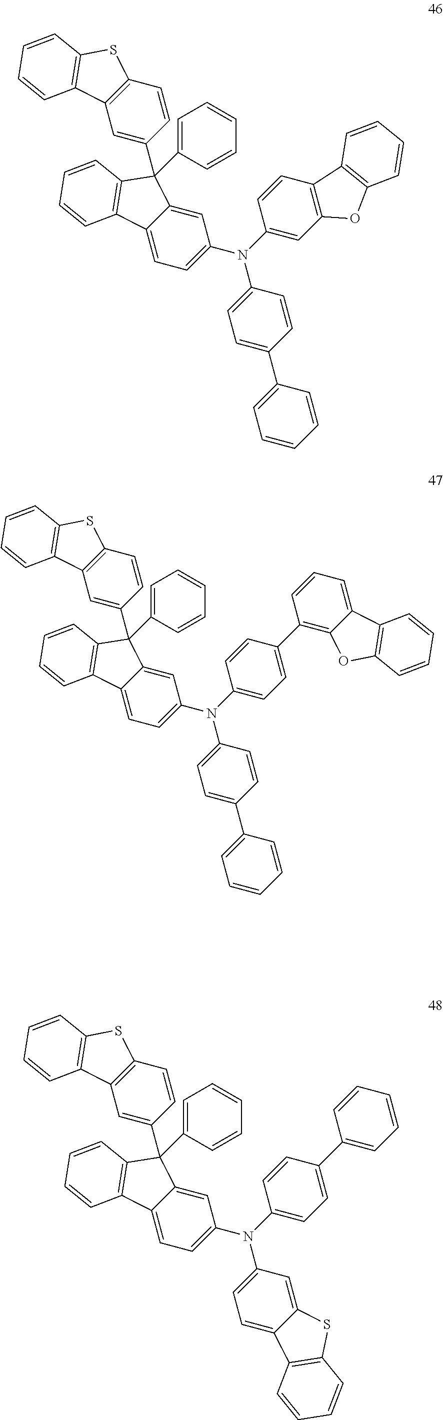 [SMILES: c1ccc(-c2ccc(N(c3ccc4c(c3)C(c3ccccc3)(c3ccc5sc6ccccc6c5c3)c3ccccc3-4)c3ccc4c(c3)oc3ccccc34)cc2)cc1 ; c1ccc(-c2ccc(N(c3ccc(-c4cccc5c4oc4ccccc45)cc3)c3ccc4c(c3)C(c3ccccc3)(c3ccc5sc6ccccc6c5c3)c3ccccc3-4)cc2)cc1 ; c1ccc(-c2ccc(N(c3ccc4c(c3)C(c3ccccc3)(c3ccc5sc6ccccc6c5c3)c3ccccc3-4)c3ccc4c(c3)sc3ccccc34)cc2)cc1] 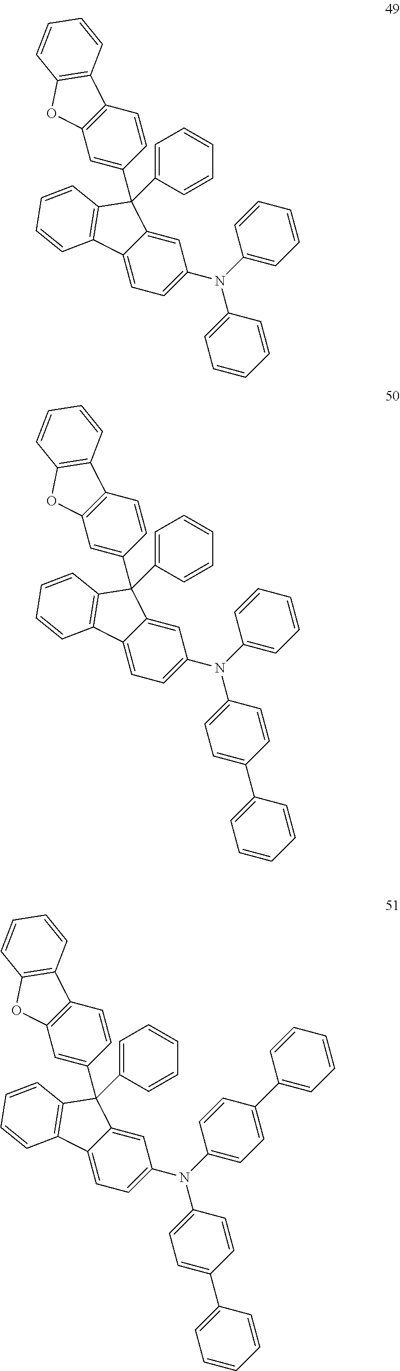 [SMILES: c1ccc(N(c2ccccc2)c2ccc3c(c2)C(c2ccccc2)(c2ccc4c(c2)oc2ccccc24)c2ccccc2-3)cc1 ; c1ccc(-c2ccc(N(c3ccccc3)c3ccc4c(c3)C(c3ccccc3)(c3ccc5c(c3)oc3ccccc35)c3ccccc3-4)cc2)cc1 ; c1ccc(-c2ccc(N(c3ccc(-c4ccccc4)cc3)c3ccc4c(c3)C(c3ccccc3)(c3ccc5c(c3)oc3ccccc35)c3ccccc3-4)cc2)cc1] 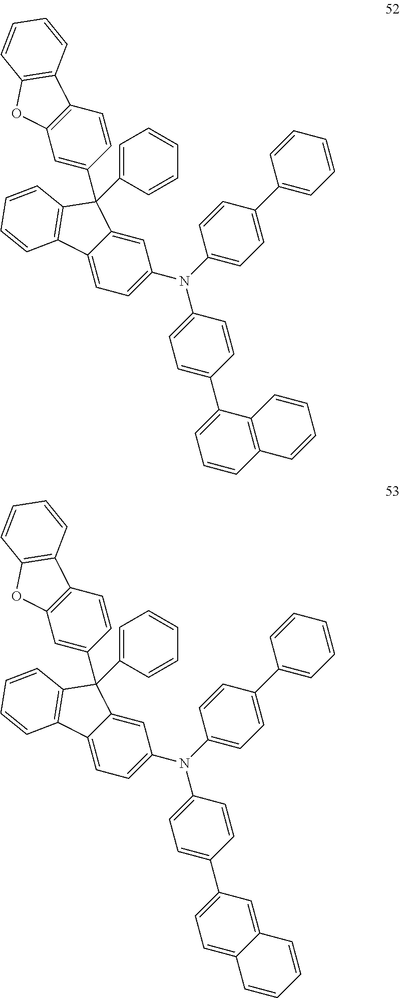 [SMILES: c1ccc(-c2ccc(N(c3ccc(-c4cccc5ccccc45)cc3)c3ccc4c(c3)C(c3ccccc3)(c3ccc5c(c3)oc3ccccc35)c3ccccc3-4)cc2)cc1 ; c1ccc(-c2ccc(N(c3ccc(-c4ccc5ccccc5c4)cc3)c3ccc4c(c3)C(c3ccccc3)(c3ccc5c(c3)oc3ccccc35)c3ccccc3-4)cc2)cc1] 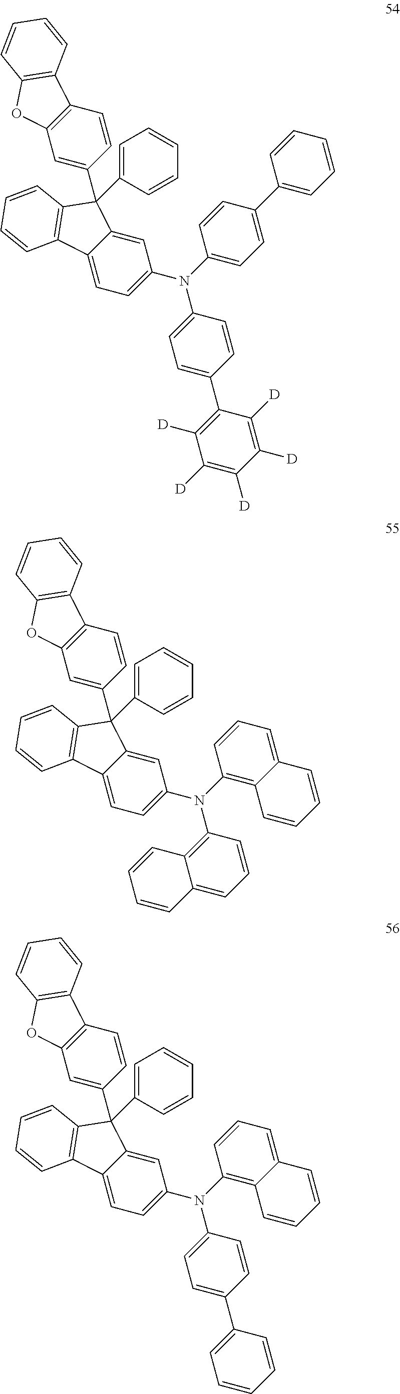 [SMILES: [2H]c1c([2H])c([2H])c(-c2ccc(N(c3ccc(-c4ccccc4)cc3)c3ccc4c(c3)C(c3ccccc3)(c3ccc5c(c3)oc3ccccc35)c3ccccc3-4)cc2)c([2H])c1[2H] ; c1ccc(C2(c3ccc4c(c3)oc3ccccc34)c3ccccc3-c3ccc(N(c4cccc5ccccc45)c4cccc5ccccc45)cc32)cc1 ; c1ccc(-c2ccc(N(c3ccc4c(c3)C(c3ccccc3)(c3ccc5c(c3)oc3ccccc35)c3ccccc3-4)c3cccc4ccccc34)cc2)cc1] 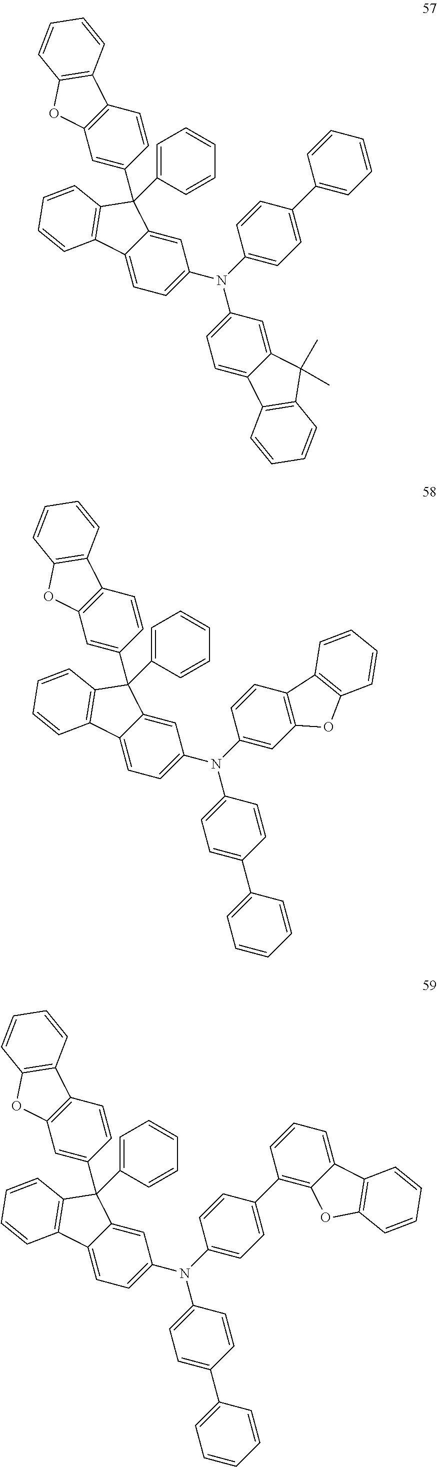 [SMILES: CC1(C)c2ccccc2-c2ccc(N(c3ccc(-c4ccccc4)cc3)c3ccc4c(c3)C(c3ccccc3)(c3ccc5c(c3)oc3ccccc35)c3ccccc3-4)cc21 ; c1ccc(-c2ccc(N(c3ccc4c(c3)C(c3ccccc3)(c3ccc5c(c3)oc3ccccc35)c3ccccc3-4)c3ccc4c(c3)oc3ccccc34)cc2)cc1 ; c1ccc(-c2ccc(N(c3ccc(-c4cccc5c4oc4ccccc45)cc3)c3ccc4c(c3)C(c3ccccc3)(c3ccc5c(c3)oc3ccccc35)c3ccccc3-4)cc2)cc1] 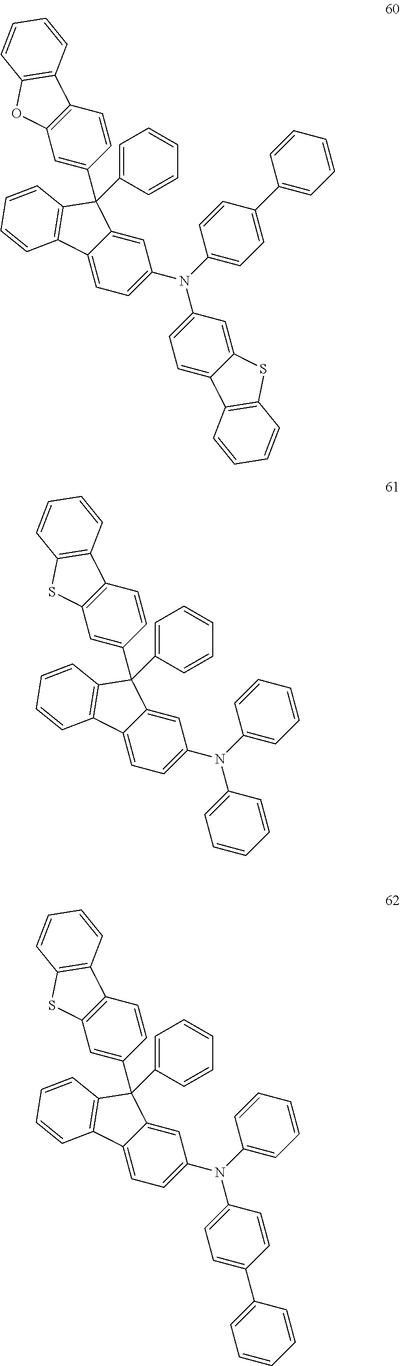 [SMILES: c1ccc(-c2ccc(N(c3ccc4c(c3)C(c3ccccc3)(c3ccc5c(c3)oc3ccccc35)c3ccccc3-4)c3ccc4c(c3)sc3ccccc34)cc2)cc1 ; c1ccc(N(c2ccccc2)c2ccc3c(c2)C(c2ccccc2)(c2ccc4c(c2)sc2ccccc24)c2ccccc2-3)cc1 ; c1ccc(-c2ccc(N(c3ccccc3)c3ccc4c(c3)C(c3ccccc3)(c3ccc5c(c3)sc3ccccc35)c3ccccc3-4)cc2)cc1] 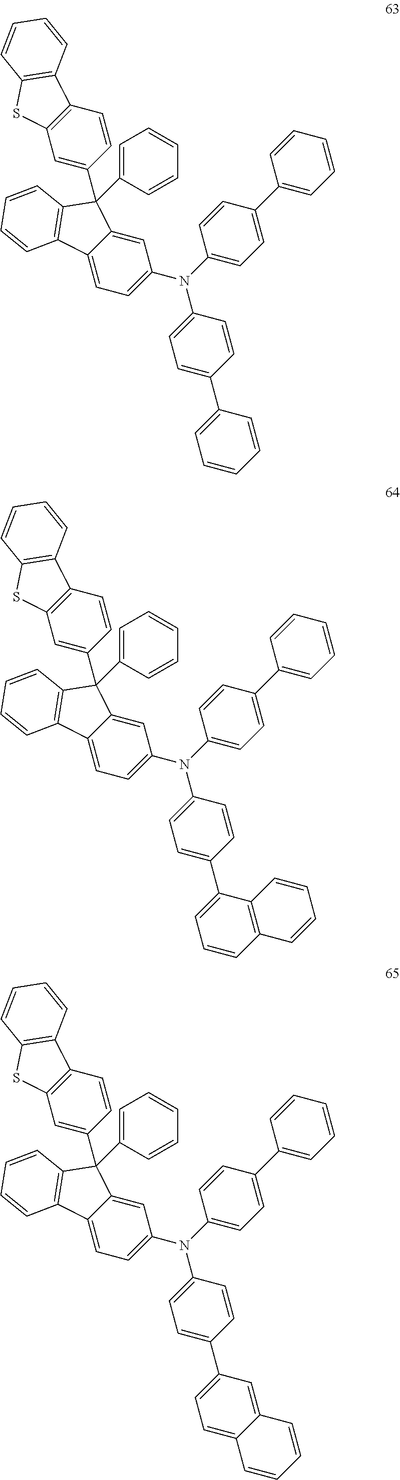 [SMILES: c1ccc(-c2ccc(N(c3ccc(-c4ccccc4)cc3)c3ccc4c(c3)C(c3ccccc3)(c3ccc5c(c3)sc3ccccc35)c3ccccc3-4)cc2)cc1 ; c1ccc(-c2ccc(N(c3ccc(-c4cccc5ccccc45)cc3)c3ccc4c(c3)C(c3ccccc3)(c3ccc5c(c3)sc3ccccc35)c3ccccc3-4)cc2)cc1 ; c1ccc(-c2ccc(N(c3ccc(-c4ccc5ccccc5c4)cc3)c3ccc4c(c3)C(c3ccccc3)(c3ccc5c(c3)sc3ccccc35)c3ccccc3-4)cc2)cc1] 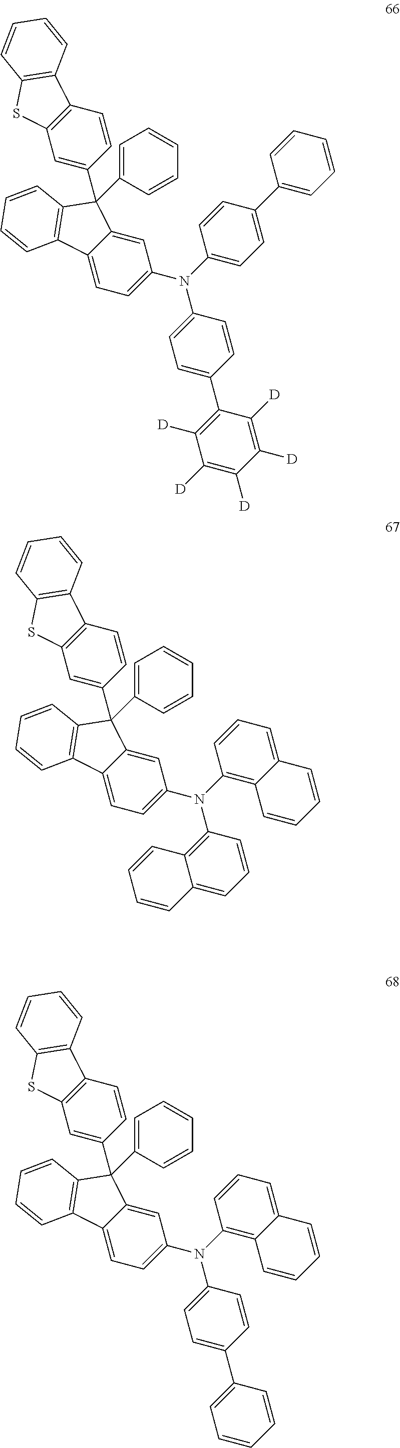 [SMILES: [2H]c1c([2H])c([2H])c(-c2ccc(N(c3ccc(-c4ccccc4)cc3)c3ccc4c(c3)C(c3ccccc3)(c3ccc5c(c3)sc3ccccc35)c3ccccc3-4)cc2)c([2H])c1[2H] ; c1ccc(C2(c3ccc4c(c3)sc3ccccc34)c3ccccc3-c3ccc(N(c4cccc5ccccc45)c4cccc5ccccc45)cc32)cc1 ; c1ccc(-c2ccc(N(c3ccc4c(c3)C(c3ccccc3)(c3ccc5c(c3)sc3ccccc35)c3ccccc3-4)c3cccc4ccccc34)cc2)cc1] 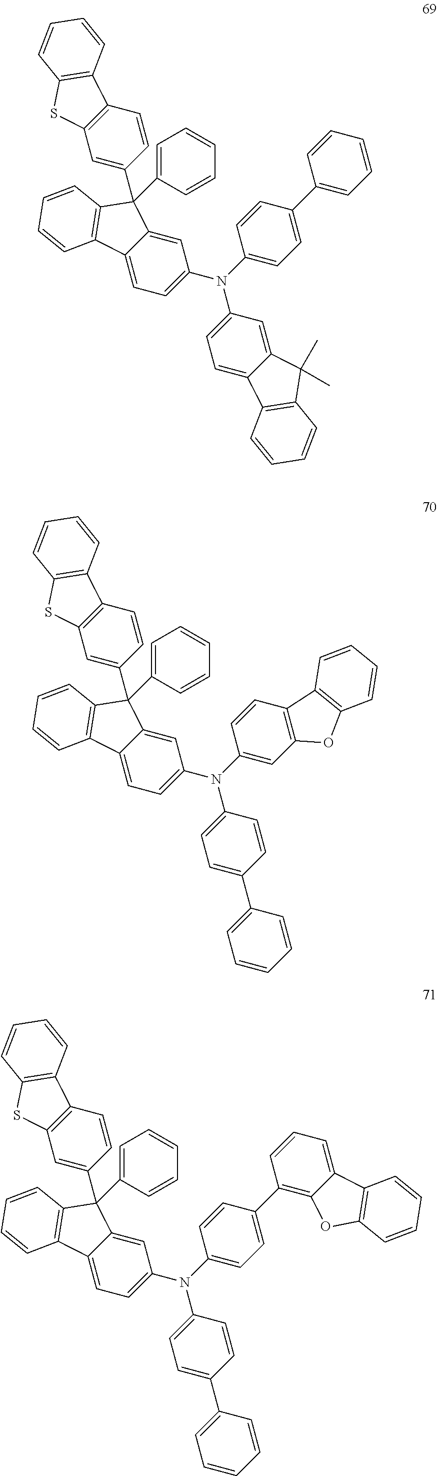 [SMILES: CC1(C)c2ccccc2-c2ccc(N(c3ccc(-c4ccccc4)cc3)c3ccc4c(c3)C(c3ccccc3)(c3ccc5c(c3)sc3ccccc35)c3ccccc3-4)cc21 ; c1ccc(-c2ccc(N(c3ccc4c(c3)C(c3ccccc3)(c3ccc5c(c3)sc3ccccc35)c3ccccc3-4)c3ccc4c(c3)oc3ccccc34)cc2)cc1 ; c1ccc(-c2ccc(N(c3ccc(-c4cccc5c4oc4ccccc45)cc3)c3ccc4c(c3)C(c3ccccc3)(c3ccc5c(c3)sc3ccccc35)c3ccccc3-4)cc2)cc1] 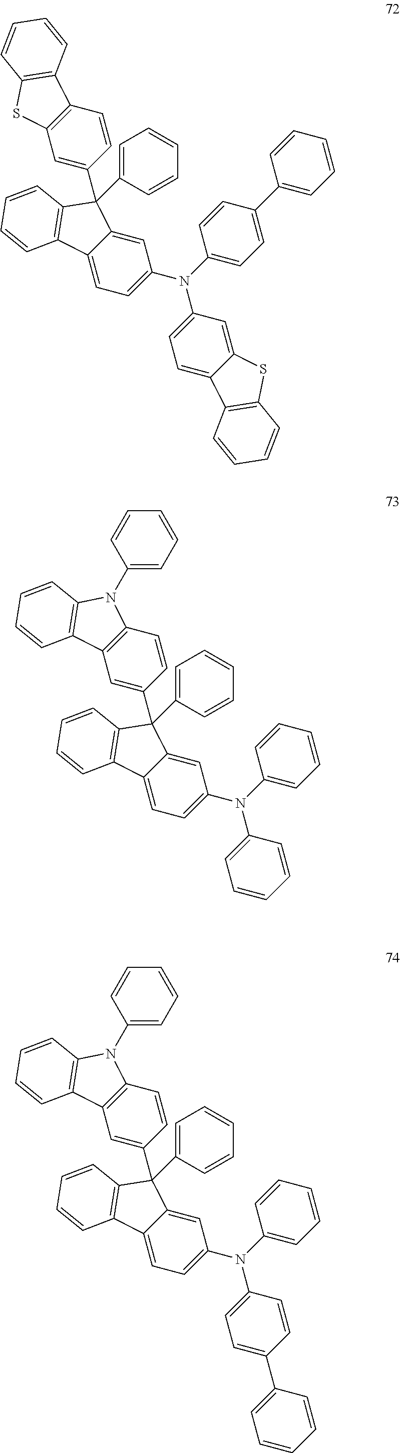 [SMILES: c1ccc(-c2ccc(N(c3ccc4c(c3)C(c3ccccc3)(c3ccc5c(c3)sc3ccccc35)c3ccccc3-4)c3ccc4c(c3)sc3ccccc34)cc2)cc1 ; c1ccc(N(c2ccccc2)c2ccc3c(c2)C(c2ccccc2)(c2ccc4c(c2)c2ccccc2n4-c2ccccc2)c2ccccc2-3)cc1 ; c1ccc(-c2ccc(N(c3ccccc3)c3ccc4c(c3)C(c3ccccc3)(c3ccc5c(c3)c3ccccc3n5-c3ccccc3)c3ccccc3-4)cc2)cc1] 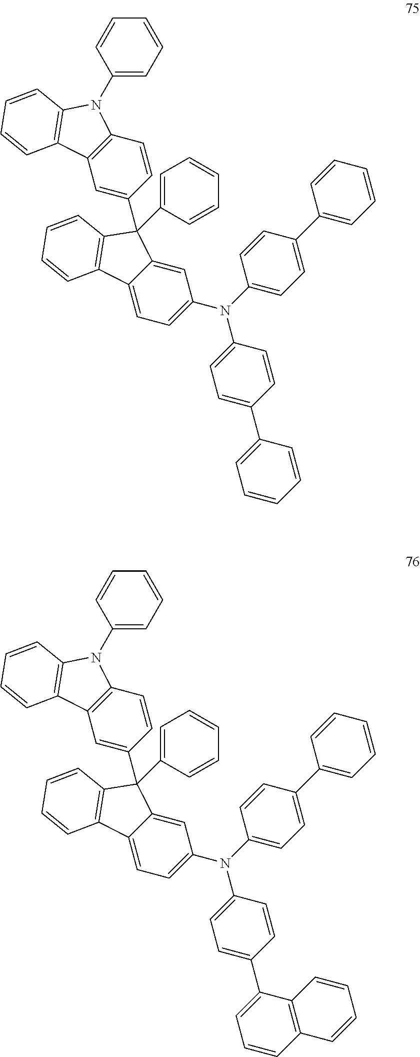 [SMILES: c1ccc(-c2ccc(N(c3ccc(-c4ccccc4)cc3)c3ccc4c(c3)C(c3ccccc3)(c3ccc5c(c3)c3ccccc3n5-c3ccccc3)c3ccccc3-4)cc2)cc1 ; c1ccc(-c2ccc(N(c3ccc(-c4cccc5ccccc45)cc3)c3ccc4c(c3)C(c3ccccc3)(c3ccc5c(c3)c3ccccc3n5-c3ccccc3)c3ccccc3-4)cc2)cc1] 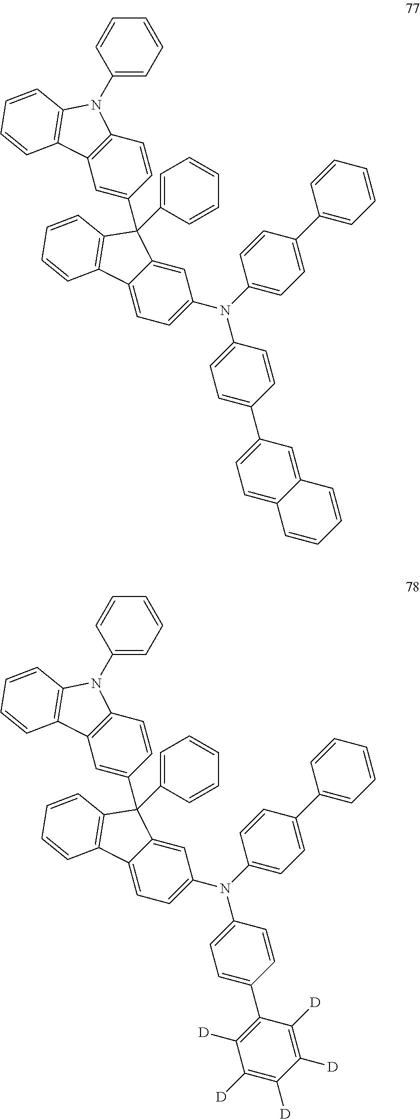 [SMILES: c1ccc(-c2ccc(N(c3ccc(-c4ccc5ccccc5c4)cc3)c3ccc4c(c3)C(c3ccccc3)(c3ccc5c(c3)c3ccccc3n5-c3ccccc3)c3ccccc3-4)cc2)cc1 ; [2H]c1c([2H])c([2H])c(-c2ccc(N(c3ccc(-c4ccccc4)cc3)c3ccc4c(c3)C(c3ccccc3)(c3ccc5c(c3)c3ccccc3n5-c3ccccc3)c3ccccc3-4)cc2)c([2H])c1[2H]] 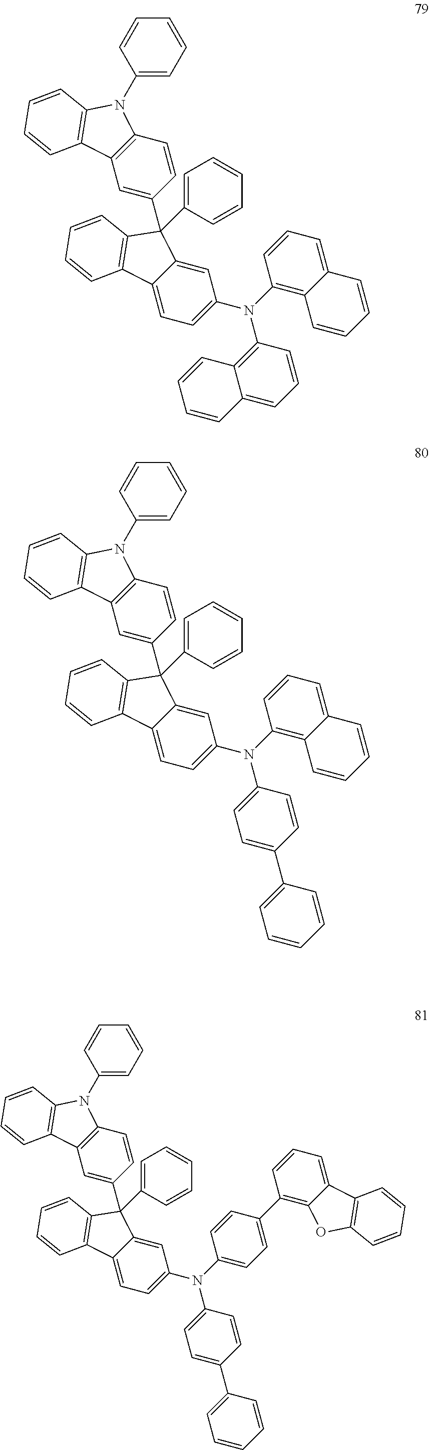 [SMILES: c1ccc(-n2c3ccccc3c3cc(C4(c5ccccc5)c5ccccc5-c5ccc(N(c6cccc7ccccc67)c6cccc7ccccc67)cc54)ccc32)cc1 ; c1ccc(-c2ccc(N(c3ccc4c(c3)C(c3ccccc3)(c3ccc5c(c3)c3ccccc3n5-c3ccccc3)c3ccccc3-4)c3cccc4ccccc34)cc2)cc1 ; c1ccc(-c2ccc(N(c3ccc(-c4cccc5c4oc4ccccc45)cc3)c3ccc4c(c3)C(c3ccccc3)(c3ccc5c(c3)c3ccccc3n5-c3ccccc3)c3ccccc3-4)cc2)cc1] 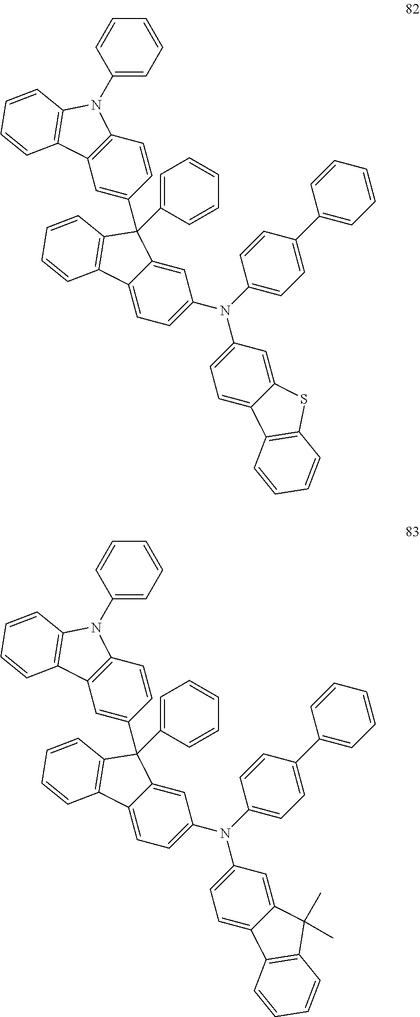 [SMILES: c1ccc(-c2ccc(N(c3ccc4c(c3)C(c3ccccc3)(c3ccc5c(c3)c3ccccc3n5-c3ccccc3)c3ccccc3-4)c3ccc4c(c3)sc3ccccc34)cc2)cc1 ; CC1(C)c2ccccc2-c2ccc(N(c3ccc(-c4ccccc4)cc3)c3ccc4c(c3)C(c3ccccc3)(c3ccc5c(c3)c3ccccc3n5-c3ccccc3)c3ccccc3-4)cc21] 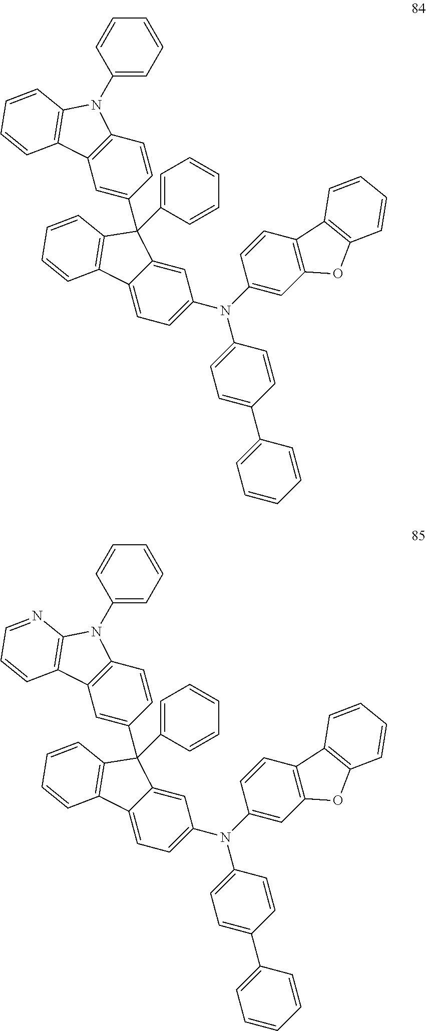 [SMILES: c1ccc(-c2ccc(N(c3ccc4c(c3)C(c3ccccc3)(c3ccc5c(c3)c3ccccc3n5-c3ccccc3)c3ccccc3-4)c3ccc4c(c3)oc3ccccc34)cc2)cc1 ; c1ccc(-c2ccc(N(c3ccc4c(c3)C(c3ccccc3)(c3ccc5c(c3)c3cccnc3n5-c3ccccc3)c3ccccc3-4)c3ccc4c(c3)oc3ccccc34)cc2)cc1] 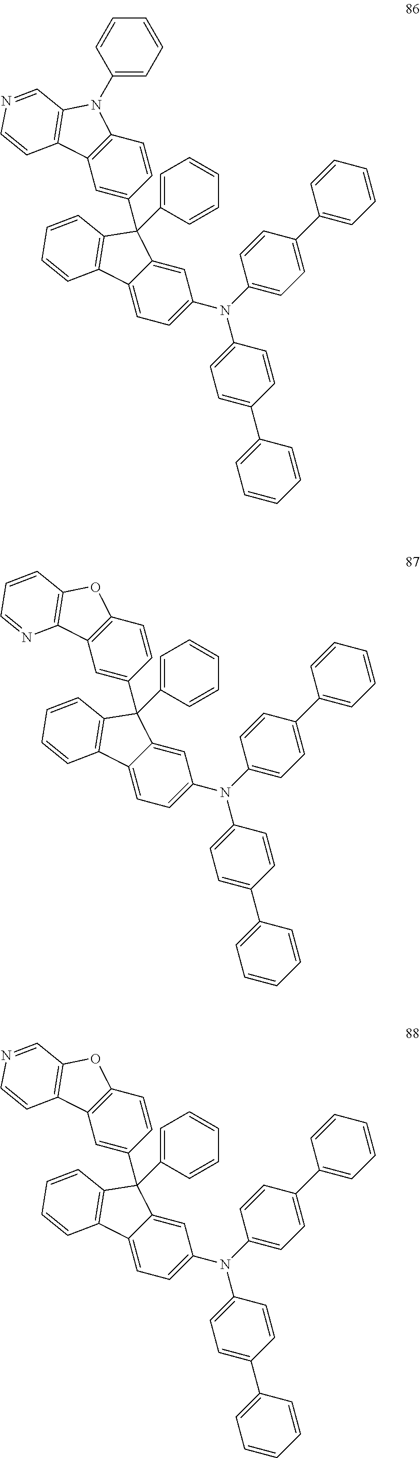 [SMILES: c1ccc(-c2ccc(N(c3ccc(-c4ccccc4)cc3)c3ccc4c(c3)C(c3ccccc3)(c3ccc5c(c3)c3ccncc3n5-c3ccccc3)c3ccccc3-4)cc2)cc1 ; c1ccc(-c2ccc(N(c3ccc(-c4ccccc4)cc3)c3ccc4c(c3)C(c3ccccc3)(c3ccc5oc6cccnc6c5c3)c3ccccc3-4)cc2)cc1 ; c1ccc(-c2ccc(N(c3ccc(-c4ccccc4)cc3)c3ccc4c(c3)C(c3ccccc3)(c3ccc5oc6cnccc6c5c3)c3ccccc3-4)cc2)cc1] 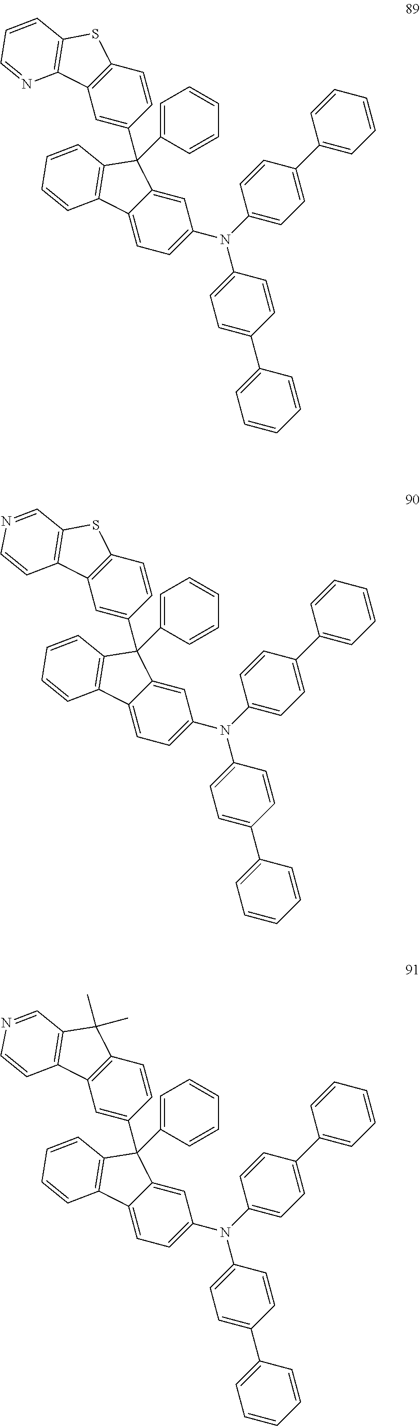 [SMILES: c1ccc(-c2ccc(N(c3ccc(-c4ccccc4)cc3)c3ccc4c(c3)C(c3ccccc3)(c3ccc5sc6cccnc6c5c3)c3ccccc3-4)cc2)cc1 ; c1ccc(-c2ccc(N(c3ccc(-c4ccccc4)cc3)c3ccc4c(c3)C(c3ccccc3)(c3ccc5sc6cnccc6c5c3)c3ccccc3-4)cc2)cc1 ; CC1(C)c2ccc(C3(c4ccccc4)c4ccccc4-c4ccc(N(c5ccc(-c6ccccc6)cc5)c5ccc(-c6ccccc6)cc5)cc43)cc2-c2ccncc21] 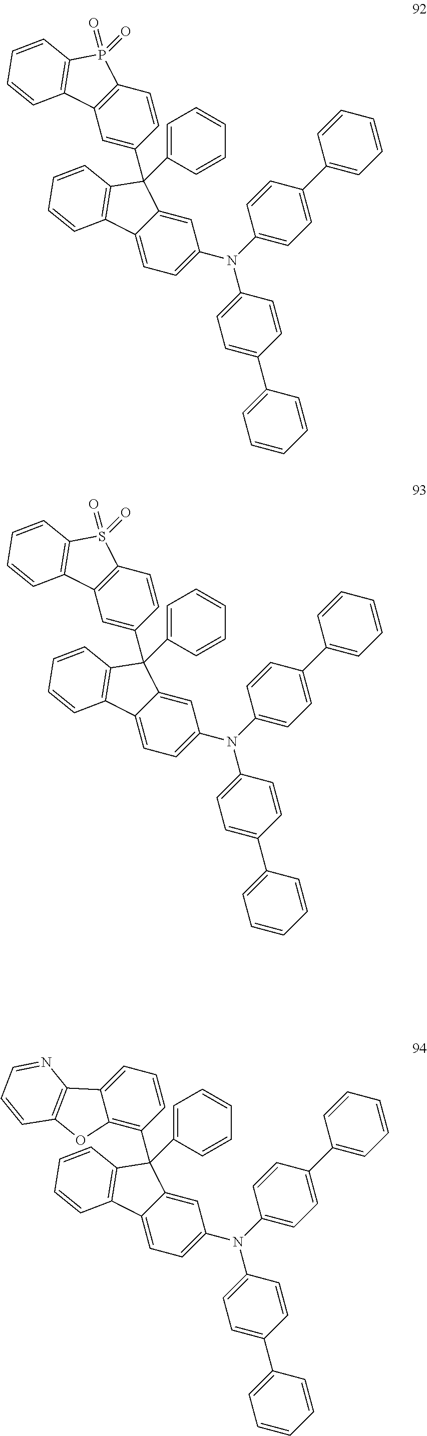 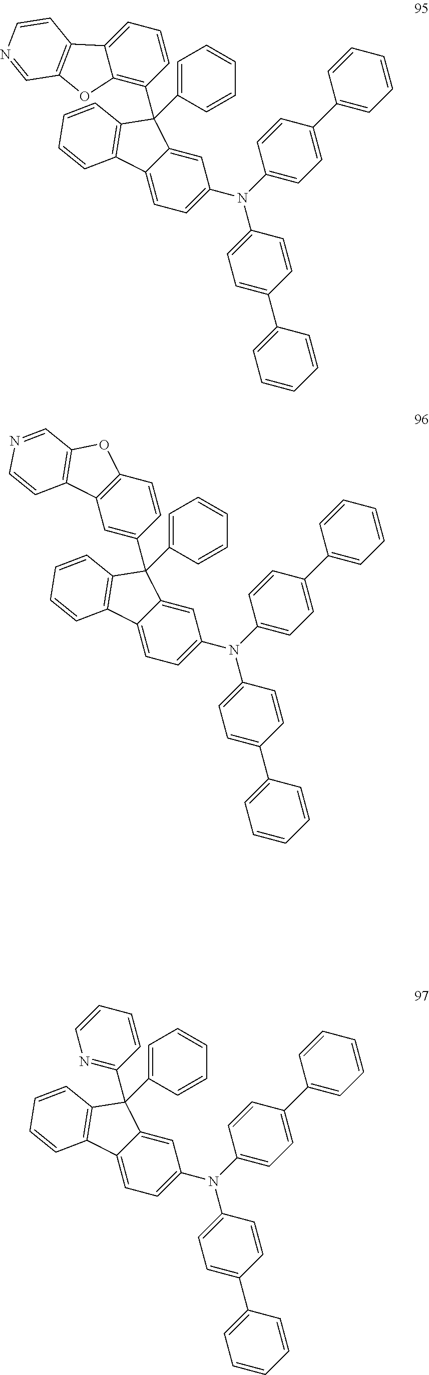 [SMILES: c1ccc(-c2ccc(N(c3ccc(-c4ccccc4)cc3)c3ccc4c(c3)C(c3ccccc3)(c3cccc5c3oc3cnccc35)c3ccccc3-4)cc2)cc1 ; c1ccc(-c2ccc(N(c3ccc(-c4ccccc4)cc3)c3ccc4c(c3)C(c3ccccc3)(c3ccc5oc6cnccc6c5c3)c3ccccc3-4)cc2)cc1 ; c1ccc(-c2ccc(N(c3ccc(-c4ccccc4)cc3)c3ccc4c(c3)C(c3ccccc3)(c3ccccn3)c3ccccc3-4)cc2)cc1] 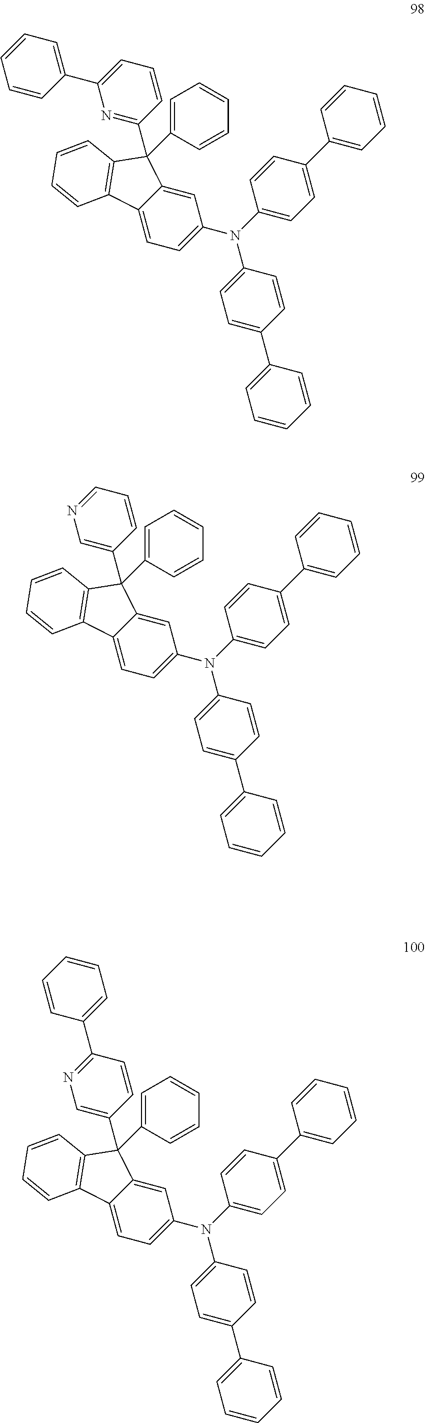 [SMILES: c1ccc(-c2ccc(N(c3ccc(-c4ccccc4)cc3)c3ccc4c(c3)C(c3ccccc3)(c3cccc(-c5ccccc5)n3)c3ccccc3-4)cc2)cc1 ; c1ccc(-c2ccc(N(c3ccc(-c4ccccc4)cc3)c3ccc4c(c3)C(c3ccccc3)(c3cccnc3)c3ccccc3-4)cc2)cc1 ; c1ccc(-c2ccc(N(c3ccc(-c4ccccc4)cc3)c3ccc4c(c3)C(c3ccccc3)(c3ccc(-c5ccccc5)nc3)c3ccccc3-4)cc2)cc1] 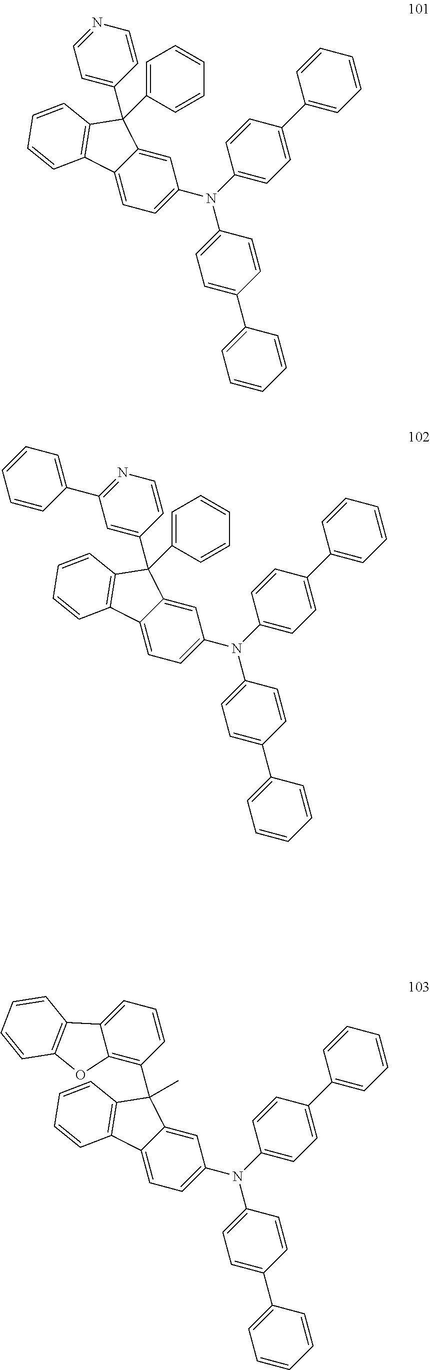 [SMILES: c1ccc(-c2ccc(N(c3ccc(-c4ccccc4)cc3)c3ccc4c(c3)C(c3ccccc3)(c3ccncc3)c3ccccc3-4)cc2)cc1 ; c1ccc(-c2ccc(N(c3ccc(-c4ccccc4)cc3)c3ccc4c(c3)C(c3ccccc3)(c3ccnc(-c5ccccc5)c3)c3ccccc3-4)cc2)cc1 ; CC1(c2cccc3c2oc2ccccc23)c2ccccc2-c2ccc(N(c3ccc(-c4ccccc4)cc3)c3ccc(-c4ccccc4)cc3)cc21] 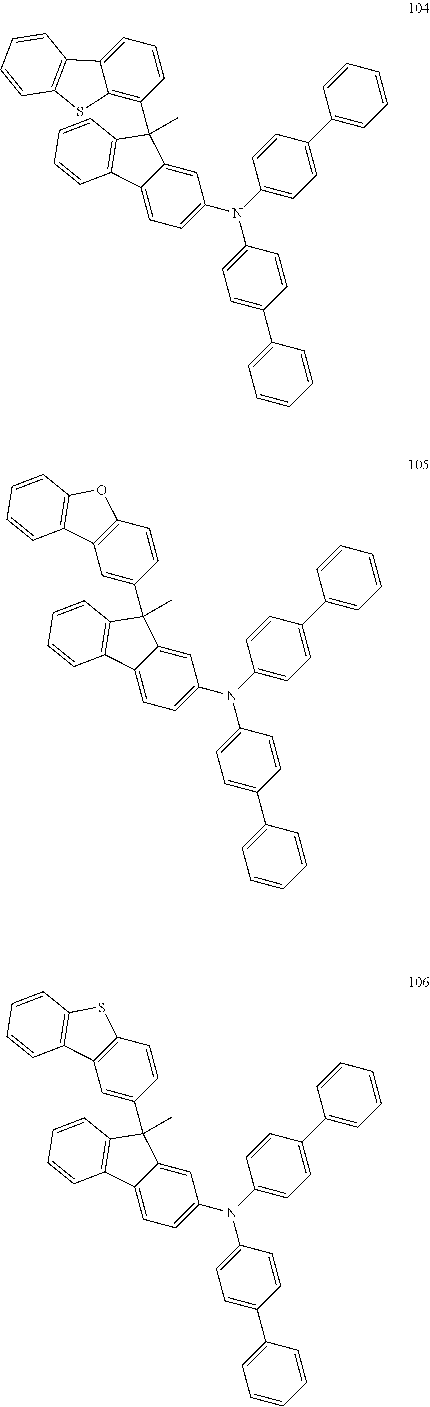 [SMILES: CC1(c2cccc3c2sc2ccccc23)c2ccccc2-c2ccc(N(c3ccc(-c4ccccc4)cc3)c3ccc(-c4ccccc4)cc3)cc21 ; CC1(c2ccc3oc4ccccc4c3c2)c2ccccc2-c2ccc(N(c3ccc(-c4ccccc4)cc3)c3ccc(-c4ccccc4)cc3)cc21 ; CC1(c2ccc3sc4ccccc4c3c2)c2ccccc2-c2ccc(N(c3ccc(-c4ccccc4)cc3)c3ccc(-c4ccccc4)cc3)cc21] 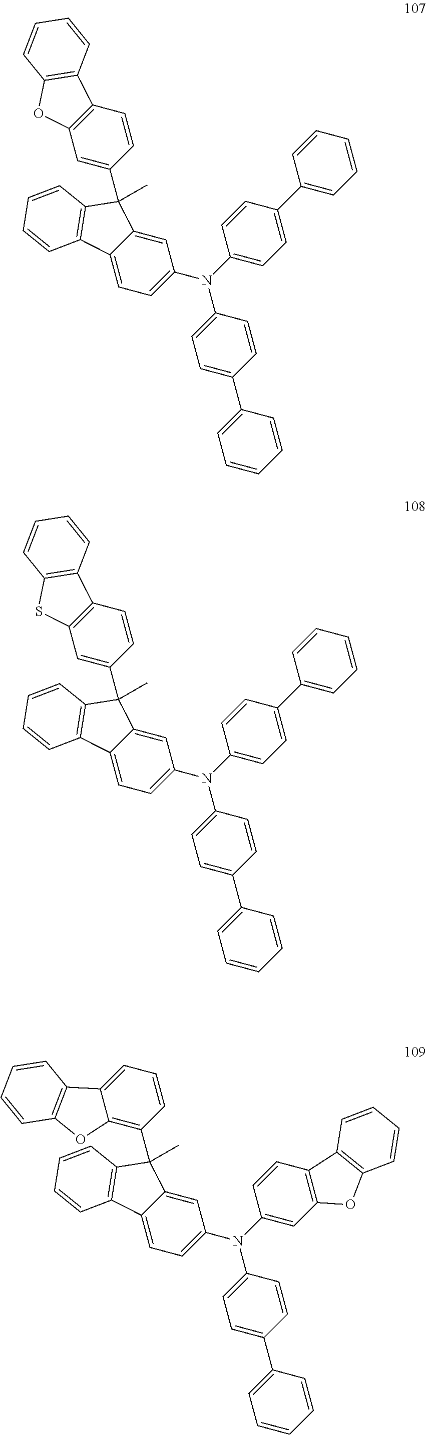 [SMILES: CC1(c2ccc3c(c2)oc2ccccc23)c2ccccc2-c2ccc(N(c3ccc(-c4ccccc4)cc3)c3ccc(-c4ccccc4)cc3)cc21 ; CC1(c2ccc3c(c2)sc2ccccc23)c2ccccc2-c2ccc(N(c3ccc(-c4ccccc4)cc3)c3ccc(-c4ccccc4)cc3)cc21 ; CC1(c2cccc3c2oc2ccccc23)c2ccccc2-c2ccc(N(c3ccc(-c4ccccc4)cc3)c3ccc4c(c3)oc3ccccc34)cc21] 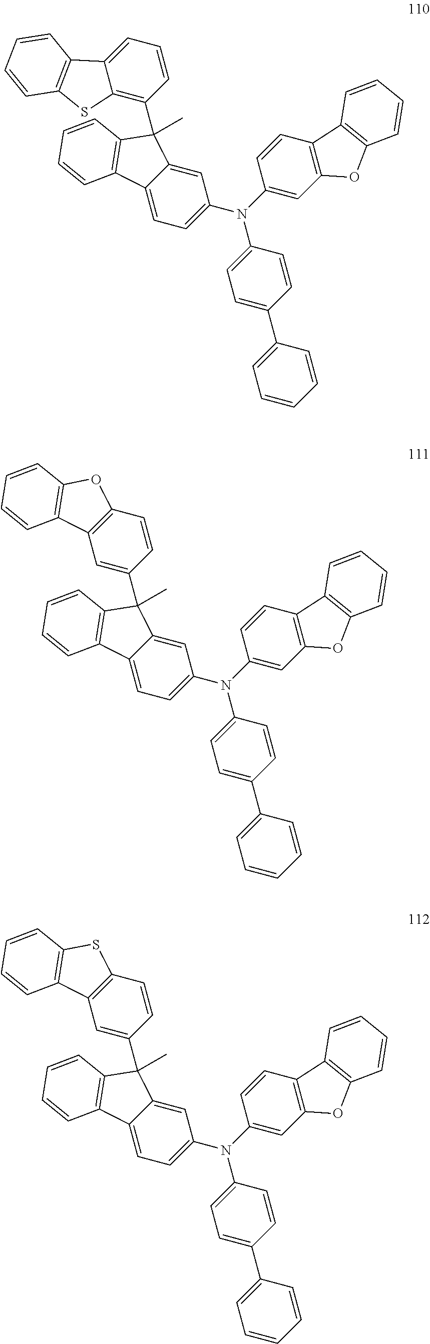 [SMILES: CC1(c2cccc3c2sc2ccccc23)c2ccccc2-c2ccc(N(c3ccc(-c4ccccc4)cc3)c3ccc4c(c3)oc3ccccc34)cc21 ; CC1(c2ccc3oc4ccccc4c3c2)c2ccccc2-c2ccc(N(c3ccc(-c4ccccc4)cc3)c3ccc4c(c3)oc3ccccc34)cc21 ; CC1(c2ccc3sc4ccccc4c3c2)c2ccccc2-c2ccc(N(c3ccc(-c4ccccc4)cc3)c3ccc4c(c3)oc3ccccc34)cc21] 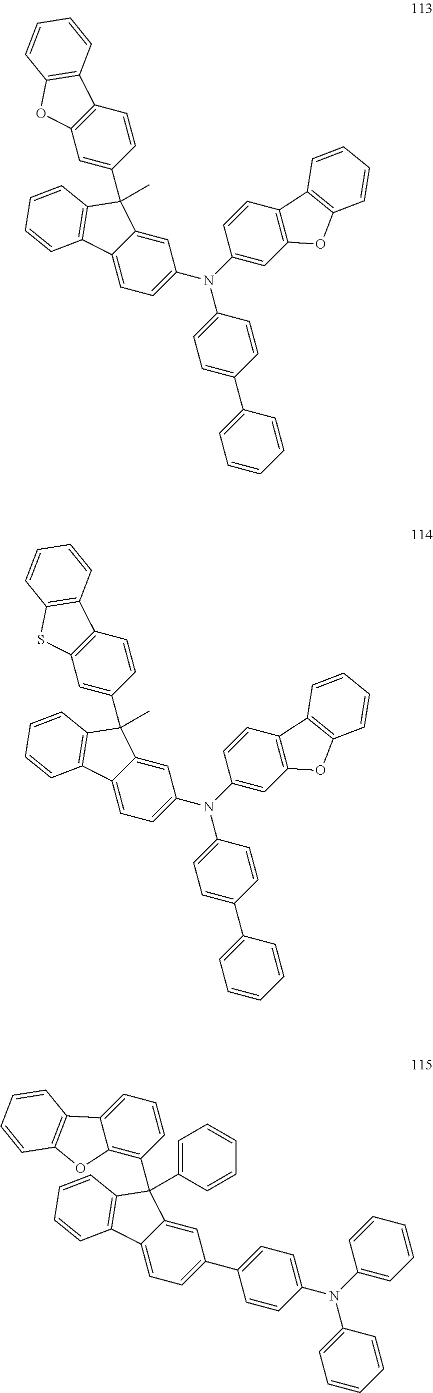 [SMILES: CC1(c2ccc3c(c2)oc2ccccc23)c2ccccc2-c2ccc(N(c3ccc(-c4ccccc4)cc3)c3ccc4c(c3)oc3ccccc34)cc21 ; CC1(c2ccc3c(c2)sc2ccccc23)c2ccccc2-c2ccc(N(c3ccc(-c4ccccc4)cc3)c3ccc4c(c3)oc3ccccc34)cc21 ; c1ccc(N(c2ccccc2)c2ccc(-c3ccc4c(c3)C(c3ccccc3)(c3cccc5c3oc3ccccc35)c3ccccc3-4)cc2)cc1] 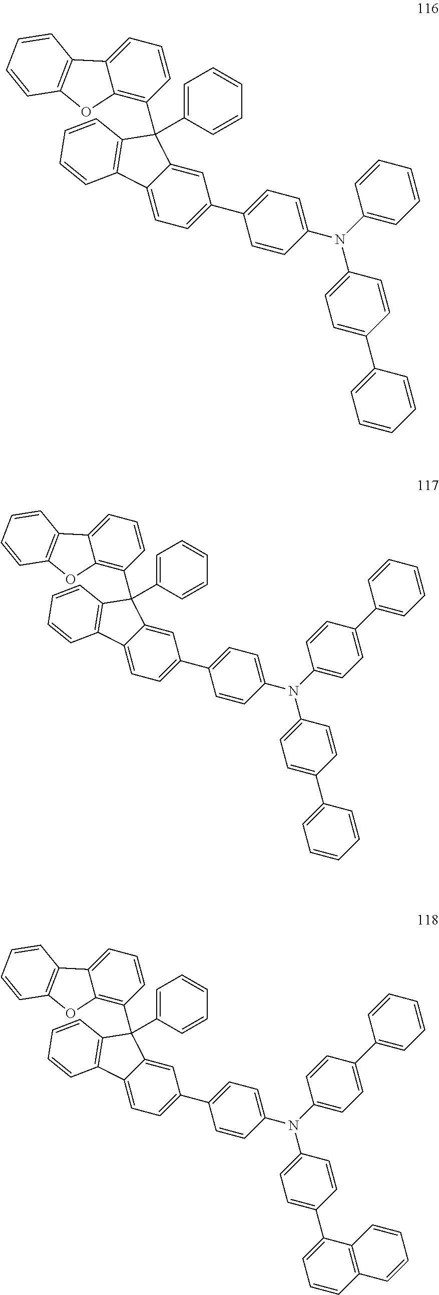 [SMILES: c1ccc(-c2ccc(N(c3ccccc3)c3ccc(-c4ccc5c(c4)C(c4ccccc4)(c4cccc6c4oc4ccccc46)c4ccccc4-5)cc3)cc2)cc1 ; c1ccc(-c2ccc(N(c3ccc(-c4ccccc4)cc3)c3ccc(-c4ccc5c(c4)C(c4ccccc4)(c4cccc6c4oc4ccccc46)c4ccccc4-5)cc3)cc2)cc1 ; c1ccc(-c2ccc(N(c3ccc(-c4ccc5c(c4)C(c4ccccc4)(c4cccc6c4oc4ccccc46)c4ccccc4-5)cc3)c3ccc(-c4cccc5ccccc45)cc3)cc2)cc1] 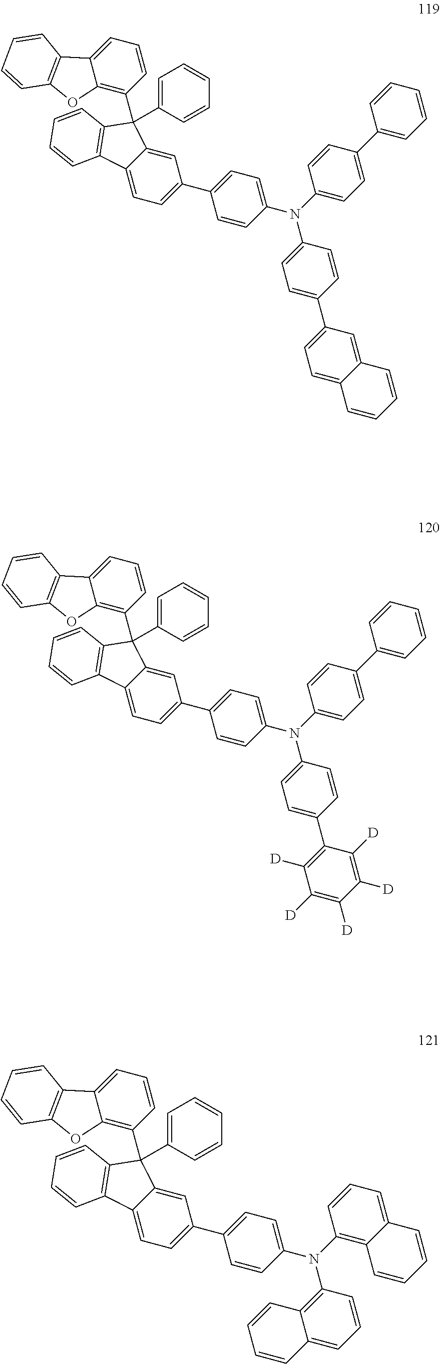 [SMILES: c1ccc(-c2ccc(N(c3ccc(-c4ccc5c(c4)C(c4ccccc4)(c4cccc6c4oc4ccccc46)c4ccccc4-5)cc3)c3ccc(-c4ccc5ccccc5c4)cc3)cc2)cc1 ; [2H]c1c([2H])c([2H])c(-c2ccc(N(c3ccc(-c4ccccc4)cc3)c3ccc(-c4ccc5c(c4)C(c4ccccc4)(c4cccc6c4oc4ccccc46)c4ccccc4-5)cc3)cc2)c([2H])c1[2H] ; c1ccc(C2(c3cccc4c3oc3ccccc34)c3ccccc3-c3ccc(-c4ccc(N(c5cccc6ccccc56)c5cccc6ccccc56)cc4)cc32)cc1] 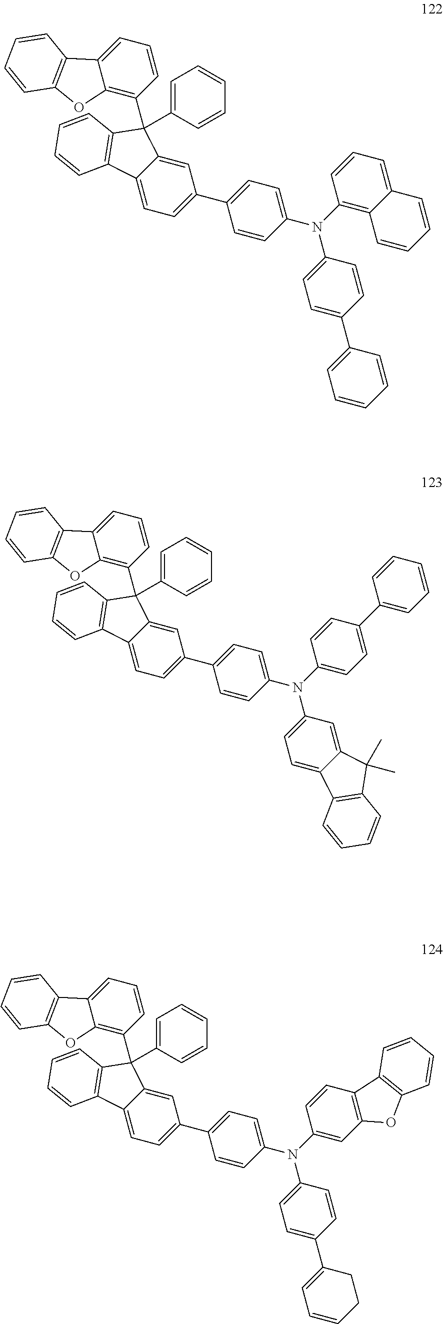 [SMILES: c1ccc(-c2ccc(N(c3ccc(-c4ccc5c(c4)C(c4ccccc4)(c4cccc6c4oc4ccccc46)c4ccccc4-5)cc3)c3cccc4ccccc34)cc2)cc1 ; CC1(C)c2ccccc2-c2ccc(N(c3ccc(-c4ccccc4)cc3)c3ccc(-c4ccc5c(c4)C(c4ccccc4)(c4cccc6c4oc4ccccc46)c4ccccc4-5)cc3)cc21 ; C1=CCCC(c2ccc(N(c3ccc(-c4ccc5c(c4)C(c4ccccc4)(c4cccc6c4oc4ccccc46)c4ccccc4-5)cc3)c3ccc4c(c3)oc3ccccc34)cc2)=C1] 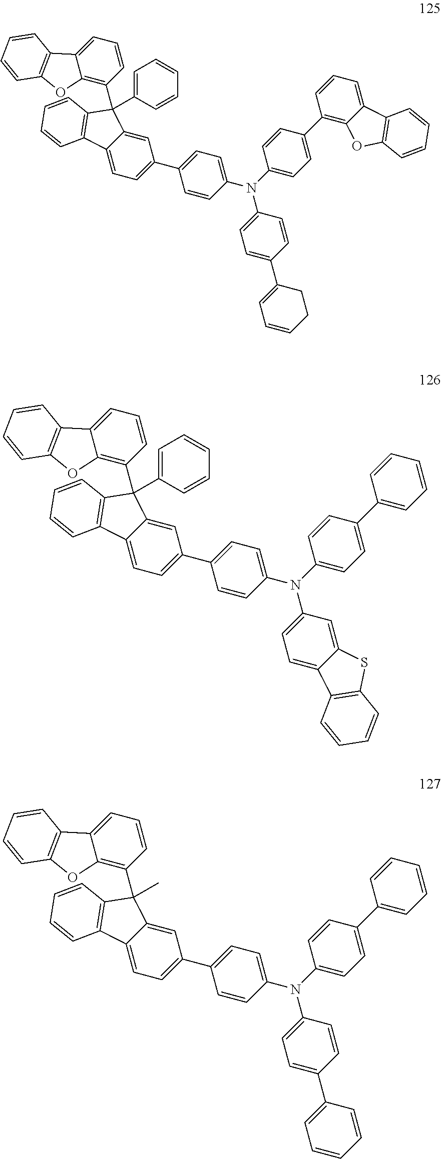 [SMILES: C1=CCCC(c2ccc(N(c3ccc(-c4ccc5c(c4)C(c4ccccc4)(c4cccc6c4oc4ccccc46)c4ccccc4-5)cc3)c3ccc(-c4cccc5c4oc4ccccc45)cc3)cc2)=C1 ; c1ccc(-c2ccc(N(c3ccc(-c4ccc5c(c4)C(c4ccccc4)(c4cccc6c4oc4ccccc46)c4ccccc4-5)cc3)c3ccc4c(c3)sc3ccccc34)cc2)cc1 ; CC1(c2cccc3c2oc2ccccc23)c2ccccc2-c2ccc(-c3ccc(N(c4ccc(-c5ccccc5)cc4)c4ccc(-c5ccccc5)cc4)cc3)cc21] 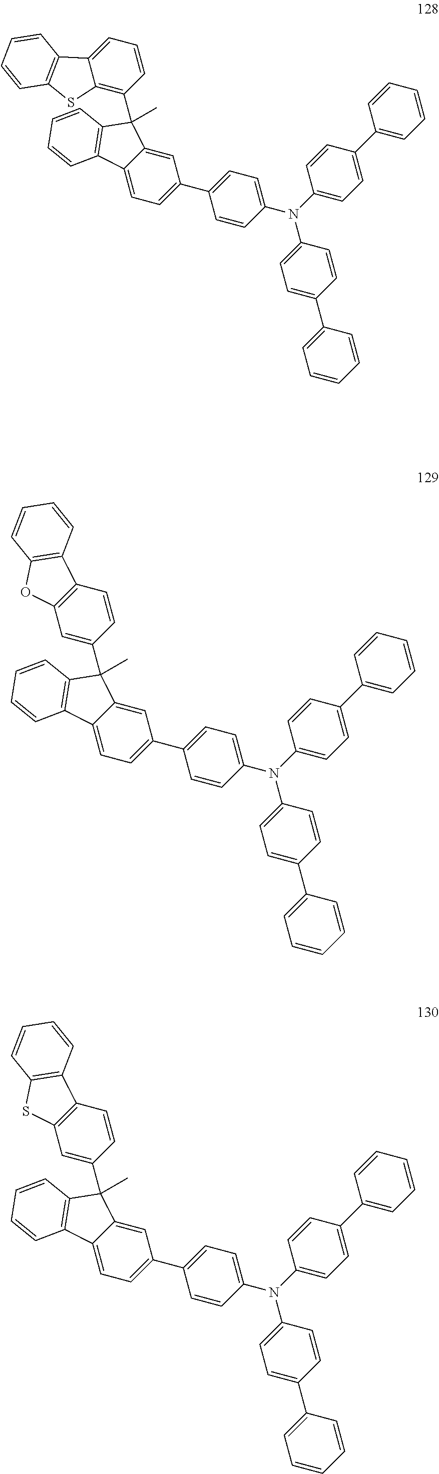 [SMILES: CC1(c2cccc3c2sc2ccccc23)c2ccccc2-c2ccc(-c3ccc(N(c4ccc(-c5ccccc5)cc4)c4ccc(-c5ccccc5)cc4)cc3)cc21 ; CC1(c2ccc3c(c2)oc2ccccc23)c2ccccc2-c2ccc(-c3ccc(N(c4ccc(-c5ccccc5)cc4)c4ccc(-c5ccccc5)cc4)cc3)cc21 ; CC1(c2ccc3c(c2)sc2ccccc23)c2ccccc2-c2ccc(-c3ccc(N(c4ccc(-c5ccccc5)cc4)c4ccc(-c5ccccc5)cc4)cc3)cc21] 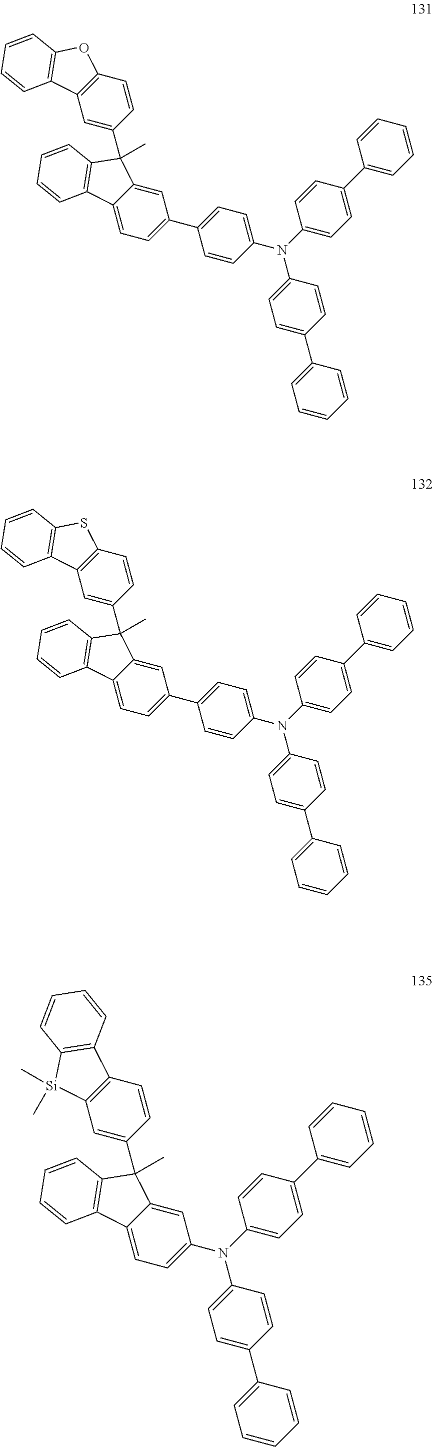 [SMILES: CC1(c2ccc3oc4ccccc4c3c2)c2ccccc2-c2ccc(-c3ccc(N(c4ccc(-c5ccccc5)cc4)c4ccc(-c5ccccc5)cc4)cc3)cc21 ; CC1(c2ccc3sc4ccccc4c3c2)c2ccccc2-c2ccc(-c3ccc(N(c4ccc(-c5ccccc5)cc4)c4ccc(-c5ccccc5)cc4)cc3)cc21 ; CC1(c2ccc3c(c2)[Si](C)(C)c2ccccc2-3)c2ccccc2-c2ccc(N(c3ccc(-c4ccccc4)cc3)c3ccc(-c4ccccc4)cc3)cc21] 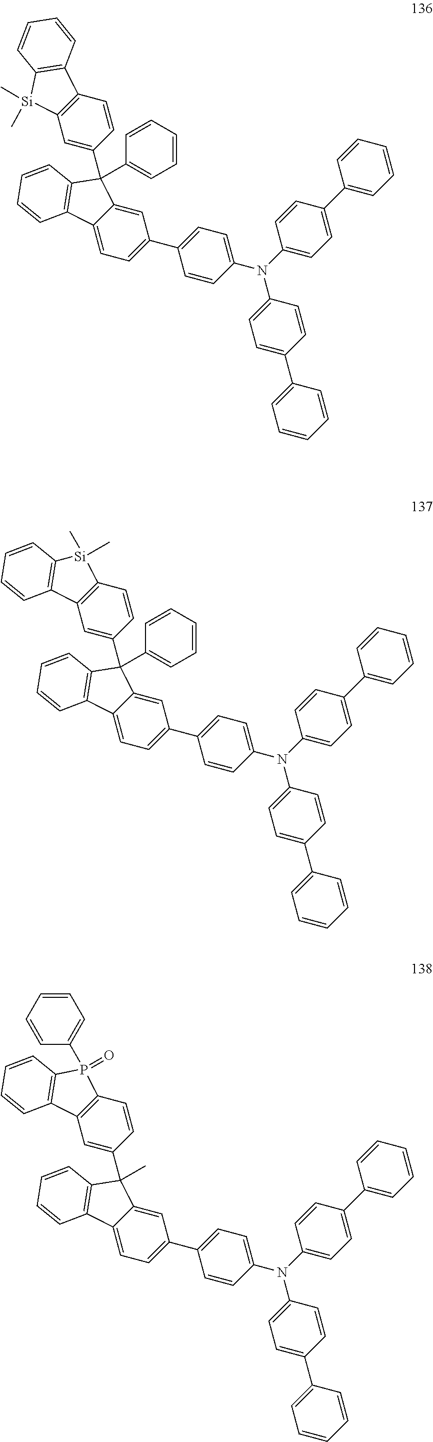 [SMILES: C[Si]1(C)c2ccccc2-c2ccc(C3(c4ccccc4)c4ccccc4-c4ccc(-c5ccc(N(c6ccc(-c7ccccc7)cc6)c6ccc(-c7ccccc7)cc6)cc5)cc43)cc21 ; C[Si]1(C)c2ccccc2-c2cc(C3(c4ccccc4)c4ccccc4-c4ccc(-c5ccc(N(c6ccc(-c7ccccc7)cc6)c6ccc(-c7ccccc7)cc6)cc5)cc43)ccc21 ; CC1(c2ccc3c(c2)-c2ccccc2P3(=O)c2ccccc2)c2ccccc2-c2ccc(-c3ccc(N(c4ccc(-c5ccccc5)cc4)c4ccc(-c5ccccc5)cc4)cc3)cc21]
